(19) United States
(12) Patent Application Publication
(10) Pub. No.: US 2004/0063751 A1
(43) Pub. Date: Apr. 1, 2004

(54) Combination therapy for the treatment of amyotrophic lateral sclerosis (ALS) with cyclooxygenase-2 (COX-2) inhibitor(s) and a second drug

(75) Inventor: Peter C. Isakson, Morris Township, NJ (US)

Correspondence Address: BANNER & WITCOFF, 1001 G STREET N W, SUITE 1100, WASHINGTON, DC, 20001, US

(73) Assignee: Pharmacia Corporation, St. Louis, MO

(21) Appl. No.: 10/444,071

(22) Filed: May 23, 2003

Related U.S. Application Data
(60) Provisional application No. 60/384,104, filed on May 31, 2002.

(51) Int. Cl. A61K 31/47; A61K 31/385; A61K 31/353

(52) U.S. Cl. 514/311; 514/314; 514/434; 514/456

(57) ABSTRACT

A method of treating, preventing, or inhibiting ALS, in a subject in need of such treatment, inhibition or prevention. The method comprises administering to a subject one or more cyclooxygenase-2 selective inhibitor(s) or isomer(s) or pharmaceutically acceptable salt(s), ester(s), or prodrug(s) thereof, in combination with one or more second drugs, wherein the amount of the cyclooxygenase-2 selective inhibitor(s) or isomer(s) or pharmaceutically acceptable salt(s), ester(s), or prodrug(s) thereof in combination with the amount of second drug(s) constitutes an ALS treatment, inhibition or prevention effective amount.

### BACKGROUND OF THE INVENTION

(1) Field of the Invention

The present invention relates to methods for the treatment of amyotrophic lateral sclerosis. More particularly, the present invention is directed to methods for the treatment of amyotrophic lateral sclerosis with cyclooxygenase-2 (COX 2) inhibitor(s) and second drug(s) in combination therewith.

(2) Description of Related Art

Amyotrophic lateral sclerosis (ALS) is one of several, clinically defined, motor neuron diseases (MNDs). ALS afflicts 1.5 times more men than women. In about two thirds of cases, the onset of the disease occurs between ages 50 and 70. James T. Caroscio, et al., “Amyotrophic Lateral Sclerosis: Its Natural History,” Neurologic Clinics, Vol. 5, No. 1, February 1987, pp. 1-8. Overall, ALS afflicts 5 to 10 people out of every 100,000 people. The progression of the disease is rapid. Most patients die within 5 years of onset. About 5-10% of ALS cases, known as familial ALS (FALS), are inherited. Although FALS is clinically indistinguishable from the “sporadic” form of ALS, there is no predominance of FALS in men as with sporadic ALS. However, the mean age of ALS onset is comparatively earlier. J. de Belleroche, et al., “Amyotrophic Lateral Sclerosis: Recent Advances in Understanding Disease Mechanisms,” J. Neuropathol. and Exp. Neurol., Vol. 55, No. 7, July 1996, pp. 747-757. No single test can diagnose ALS. Because of the slow onset of the disease, diagnosis of ALS is usually difficult in its early stages. By the time of positive diagnosis, the disease has generally progressed for 1-2 years.

ALS patients manifest symptoms associated with the loss of motor neurons, and/or the nerve cells in the spinal cord, brain stem, and motor cortex, which are normally in good control of the body's voluntary muscles. In ALS, as motor neurons die, muscles weaken and shrink, and the body manifests the early-stage symptoms of ALS. Such symptoms include, for example, unusual fatigue, clumsiness, muscle weakness, slurred speech, muscle atrophy, spasticity, spinal function disorders, and convulsions. As ALS progresses, patients gradually lose the use of their hands, arms, legs, and neck muscles, ultimately becoming paralyzed. Speaking and swallowing ability are greatly compromised. Psychiatric manifestations (e.g., depression) may also result.

However, while cognitive impairment is generally not observed with ALS, some data suggest that as many as 15% of all ALS patients may experience some memory loss, behavioral changes, and problems with both judgment and simultaneously performing multiple tasks. 12^{th} International Symposium on ALS/MND, Oakland, Calif., Nov. 18-20, 2001, Highlights and Summary of Clinical Sessions, (www.alsa.org/news/news120301a.cfm). The usual cause of death from ALS is failure of the diaphragm muscles that control breathing. ALS patients can prolong their lives by using a ventilator, especially since bladder and bowel function, sexual function, and all five senses are unaffected. But living on a ventilator is neither desirable nor free of complications such as pneumonia (resulting from pooling of secretions or aspiration).

Evidence from early research into possible causes of ALS does not support either viral or environmental toxin theories. Also, while several autoimmune theories have been advanced in the past decade, immunosuppressive therapy, including drug (e.g., azathioprine) treatment, plasmapheresis, or intravenous immunoglobulin injection, has been ineffective in combating ALS. Even the very potent immunosuppression method of total lymph node irradiation proved unsuccessful. Annals of Neurology, Editorial, “Amyotrophic Lateral Sclerosis: Theories and Therapies,” Vol. 35, 1994, pp. 129-130. Currently, one area of ongoing investigation is the use of growth factors, such as Insulin-like growth factor 1 (IGF-1 or Myotrophin®), ciliary neurotrophic factor (CNTF), and, most recently, vascular epithelial growth factor (VEGF) that have shown protection of motor neurons in animal models and cell culture systems. The ALS Association has announced enrollment, beginning in late winter 2002, for a clinical research trial of IGF-1. 12^{th} International Symposium on ALS/MND, Oakland, Calif., Nov. 18-20, 2001, Highlights and Summary of Clinical Sessions, (www.alsa.org/news/ews120301a.cfm). The effectiveness of growth factor therapy, however, may be limited by the degradation of the protein in the liver, before it crosses the blood brain barrier (BBB).

Another avenue of therapy is in the regulation of brain glutamate levels, based on abnormally high glutamate concentrations found in cerebrospinal fluid of some ALS patients. An abundance of the glutamate transporter in astrocytes (cells surrounding the neurons) known as EAAT2, involved in the removal of excess glutamate, is decreased in the cortex and spinal cord of patients with ALS and in mouse models. A high glutamate level leads to “excitotoxicity” (which is the activation of glutamate receptors), a flooding of neurons with calcium, and a host of damaging downstream events. Other studies have linked excitotoxicity to high zinc levels as a cause of motor neuron death. Id. From these findings, a promising area of investigation is in the use of glutamate antagonists, which inhibit the release of glutamate in the brain. Preliminary results with the FDA-approved drug riluzole (Rilutek®) indicate potential for increasing the life expectancy of ALS patients. However, double blind and controlled studies are needed to confirm these observations. Id. One drawback of riluzole therapy is the need for regular monitoring for potentially high levels of liver enzymes, which are selected may require discontinuing riluzole drug therapy. Other glutamate antagonists of interest include dextromethorphan and lamotrigine. As with all such ALS drugs, their potential to degrade before crossing the BBB remains a significant concern.

Finally, the identification of mutations in the gene encoding for the enzyme superoxide dismutase (SOD) in FALS has been a landmark in ALS research. SOD mutations are present in 15-20% of FALS cases. SOD converts the superoxide free radical anion (O²⁻) to hydrogen peroxide (H₂O₂), which is further detoxified by enzymes such as catalase. The importance of SOD in handling free radicals during oxidative stress has been recognized for some time, leading some scientists to believe that mutant SOD may result in motor neuron destruction through the action of an excessive amount of free radicals. Studies of SOD treatment alone or in combination with other drugs such as riluzole for the treatment of both sporadic and familial ALS are ongoing.

Under certain conditions of chronic neurodegeneration, neuroinflammation may be observed. However, the functional consequences of chronic inflammatory processes in the brain are not well understood.

Recently, compounds that selectively inhibit cyclooxygenase-2 have been discovered. These COX 2 inhibiting compounds selectively inhibit the activity of COX 2 to a greater extent than the activity of cyclooxygenase-1 (COX 1). COX 1 has been shown to be a constitutively produced enzyme that is involved in many of the non-inflammatory regulatory functions associated with prostaglandins. COX 2, on the other hand, is an inducible enzyme having significant involvement, in the inflammatory process. See, Needleman, P. et al, J. Rheumatol., 24, Suppl.49:6-8 (1997). See, Fu, J. Y., et al., J. Biol. Chem., 265(28):16737-40 (1990). The new COX 2-selective inhibitors are believed to offer advantages that include avoiding harmful side effects associated with the inhibition of COX 1.

Information on the identification and/or use of cyclooxygenase-2-selective inhibitors can be found in references such as: (1) Buttgereit, F. et al., Am. J. Med., 110(3 Suppl. 1):13-9 (2001); (2) Osiri, M. et al, Arthritis Care Res., 12(5):351-62 (1999); (3) Buttar, N. S. et al., Mayo Clin. Proc., 75(10):1027-38 (2000); (4) Wollheim, F. A., Current Opin. Rheumatol., 13:193-201 (2001); (5) U.S. Pat. Nos. 5,434,178 (1,3,5-trisubstituted pyrazole compounds); (6) 5,476,944 (derivatives of cyclic phenolic thioethers); (7) 5,643,933 (substituted sulfonylphenylheterocycles); 5,859,257 (isoxazole compounds); (8) 5,932,598 (prodrugs of benzenesulfonamide-containing COX 2 inhibitors); (9) 6,156,781 (substituted pyrazolyl benzenesulfonamides); (10) 6,110,960 (for dihydrobenzopyran and related compounds), (11) 6,180,651 (includes disclosure of BMS-347070), (12) Hillson, J. L. et al., Expert Opin. Pharmacother., 1(5):1053-66 (2000), (for rofecoxib, Vioxx®, Merck & Co., Inc.); (13) Everts, B. et al., Clin. Rheumatol., 19(5):331-43 (2000), (for celecoxib, Celebrex®, Pharmacia Corporation, and rofecoxib); (14) Jamali, F., J. Pharm. Pharm. Sci., 4(1):1 - 6 (2001), (for celecoxib); (15) U.S. Pat. Nos. 5,521,207 and 5,760,068 (for substituted pyrazolyl benzenesulfonamides); (16) Davies, N. M. et al., Clinical Genetics, Abstr. at http://www.mmhc.com/cg/articles/CG0006/davies.html (for meloxicam, celecoxib, valdecoxib, parecoxib, deracoxib, and rofecoxib); (17) http://www.celebrex.com (for celecoxib); (18) http://www.docguide.com/dg.nsf/PrintPrint/F1F8DDD2D8B0094085256 98F00742187, May 9, 2001 (for etoricoxib, MK-663, Merck & Co., Inc.); (19) Saag, K. et al., Arch. Fam. Med., 9(10):1124 - 34 (2000), (for rofecoxib); and (20) International Patent Publication No. WO-0024719 (for ABT 963, Abbott Laboratories).

Various U.S. patents and patent applications discuss the treatment of a number of neurodegenerative diseases as well as other diseases. For example, (21) U.S. Pat. No. 6,306,842 B1 teaches the use of a compound of the structure X-L-Y, wherein X is non-steroidal anti-inflammatory drug (NSAID), L is an optional linker/spacer, and Y is a selective COX 2 inhibitor; (22) U.S. Pat. No. 6,303,613 B1 teaches the use of compounds, alone or in combination with a COX 2 inhibitor (e.g., celecoxib or MK-966), of the formula:

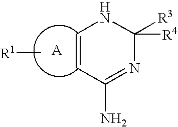

wherein A represents a five membered heterocyclic aromatic ring containing 1 to 3 heteroatoms which may be the same or different and are selected from O, N, and S; or a six membered heterocyclic aromatic ring containing 1 to 3 nitrogen atoms; R′ represents hydrogen, alkyl C₁ to C₆, alkoxy C₁ to C₆, halogen or trifluoromethyl;

R² represents hydrogen or alkyl C1 to 6; and R³ and R⁴ are as defined in the specification; (23) U.S. Pat. No. 6,294,170 teaches the use of a COX 2 inhibitor (e.g. celecoxib) in combination with an IL-1 inhibitor protein, thought to mediate various conditions; (24) U.S. Pat. Nos. 6,265,436 B1, 6,262,073 B1, 6,136,832, and 6,005,000, teach the use of certain 5,5-disubstituted-3,4-dihydroxy-2(5H)-furanones and certain substituted 5-biphenyl-3,4-dihydroxy-2(5H)-furanones compounds having the generalized formulas

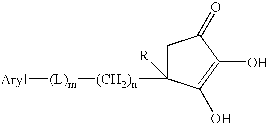

wherein R is hydrogen, phenyl, or lower alkyl; L is a linker moiety selected from the group consisting of oxygen, sulfur, nitrogen, acetylene, a cis or trans carbon-carbon double bond, an ester, carbonate, urea, amide, and carbamate; m is 0 or 1; n is 0 to 4; Aryl is a substituted or unsubstituted aryl group; with the proviso that when R is hydrogen, then either m or n is not zero, and the pharmaceutically acceptable salts thereof;

and

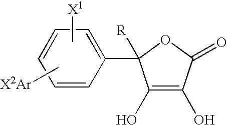

wherein R is hydrogen, a lower alkyl group optionally substituted by one or more halo groups, a cycloalkyl group, or an aryl group optionally substituted by one or more halo, alkyl of one to eight carbon atoms, alkoxy of one to eight carbon atoms, cycloalkyl, nitro or trifluoromethyl groups; X¹ is optionally one or more halo, alkyl of one to eight carbon atoms, alkoxy of one to eight carbon atoms, cycloalkyl, nitro or trifluoromethyl groups; and Ar is an aromatic or heteroaromatic ring substituted by X², X² being one or more halo, alkyl of one to eight carbon atoms, alkoxy of one to eight carbon atoms, cycloalkyl, nitro or trifluoromethyl groups; or a pharmaceutically acceptable salt thereof; (27) U.S. Pat. No. 6,063,807 teaches the use of a salt AB where A is a cyclooxygenase inhibitor and B is a compound of the formula

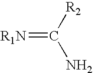

wherein R₁ represents H, the nitro or phenyl radical, the phenyl radical potentially being substituted by one or more substituents chosen from among the halo, cyano, nitro, trifluorormethyl, lower alkyl or lower alkoxy radicals; R₂ represents a lower alkyl radical; lower alkylthio; alkylthioalkyl; aryl potentially substituted by one or more substituents chosen from among the halo, cyano, nitro, trifluoromethyl, lower alkyl or lower alkoxy radicals; or amino potentially substituted by a radical chosen from among the nitro, amino, lower alkyl or phenyl radicals, the phenyl radical itself potentially being substituted by one or more substituents chosen from among the halo, cyano, nitro, trifluoromethyl, lower alkyl or lower alkoxy radicals; and when A represents acetylsalicylic acid and R₁ a hydrogen atom, then R₂ represents neither an aryl radical nor a phenylamino radical, the phenyl radical potentially being substituted; and, (28) U.S. Published application No. 2001/0011143A1 teaches the use, in combination with COX 2 inhibitors, inhibitors of matrix metalloproteinase (MMP) of the formulas

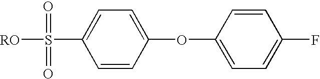

wherein R is H, Li, Na, K, Mg, or NH₄;

and

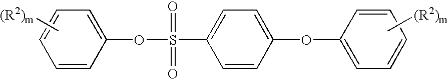

wherein m is an integer from 1-3; R² is fluoro, chloro, bromo, (C₁-C₆)alkyl, (C₁-C₆)alkoxy or perfluoro(C₁-C₃)alkyl.

In summary, the search for an effective therapeutic agent for ALS is currently based on a broad range of theories, including those discussed above, regarding the pathogenesis of the disease. However, despite the varied approaches, the area is distinguished by many inconclusive or disappointing results. Harriet M. Bryson, et al., “Riluzole: A Review of its Pharmacodynamic and Pharmokinetic Properties and Therapeutic Potential in Amyotrophic Lateral Sclerosis,” Drugs, October 1996 (52(4): 549-563. Methods tried without conclusive benefit include the use of antioxidants (e.g. acetylcysteine), immunotherapeutic techniques (e.g., lymphoid irradiation), growth factors (e.g., ciliary neurotrophic factor), and agents that modify glutamate transmission (e.g., lamotrigine) or metabolism (e.g., branched chain amino acids). Against this background, there is a need to find other agents and methods for treating ALS.

### SUMMARY OF THE INVENTION

According to one embodiment, the invention is directed to a novel method for the treatment, inhibition and/or prevention of ALS (and/or its symptoms) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, wherein said COX 2 inhibitor comprises a chromene that is a substituted benzopyran or is a chroman.

According to another embodiment, the invention is directed to a novel method for the treatment, inhibition and/or prevention of ALS comprising administering, to a subject in need thereof, a therapeutically effective amount of cyclooxygenase-2 selective inhibitor which is I, II, III, IV, V, B-1, B-2, . . . B-232, or B-233 or combination(s) thereof (or an isomer, a pharmaceutically acceptable salt, ester, or prodrug thereof, respectively), and a second drug which is C-1, C-2, C-3, C-4 . . . C-180 or C-181 (or an isomer, a pharmaceutically acceptable salt, ester, or prodrug thereof, respectively), or combination(s) thereof. See Tables 1, 1A, 2, 2A and 2B below. COX 2 inhibitors suitable for use with the present inventive method include, but are not limited to, those COX 2 inhibitors disclosed in Tables 1 and 1A below. Second drugs suitable for use in combination with COX 2 inhibitors according to the present inventive method include, but are not limited to, those disclosed in Table 2 below. Combinations of COX 2 inhibitors and second drugs for use according to the present invention include, but are not limited to, those combinations set forth in Tables 2A and 2B below.

TABLE 1

| No. | Structure (COX 2 Inhibitor) |
|---|---|
| I | 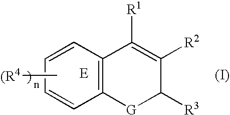 |
| | wherein R¹, R², R³, R⁴, n and G are as described herein. |
| II | 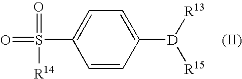 |
| | wherein R¹³, R¹⁴, R¹⁵, and D are as described herein. |
| III | 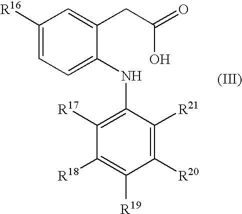 |
| | wherein R¹⁶, R¹⁷, R¹⁸, R¹⁹, R²⁰, and R²¹, are as described herein. |
| IV | 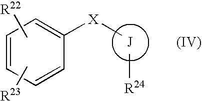 |
| | wherein R²², R²³, R²⁴, X and J are as described herein. |
| V | 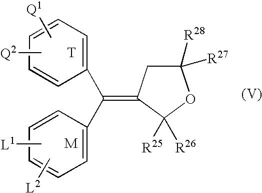 |
| | wherein Q¹, Q², L¹, L², R²⁵, R²⁶, R²⁷ and |
| | R²⁸ are as described herein. |

TABLE 1A

| First | |
|---|---|
| Drug | Name and/or Structure (COX 2 Inhibitor) |
| B-1 | 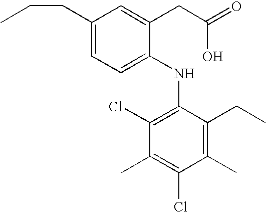 |
| | [2-(2,4-Dichloro-6-ethyl-3,5-dimethyl-phenylamino)- |
| | 5-propyl-phenyl]-acetic acid; |
| B-2 | 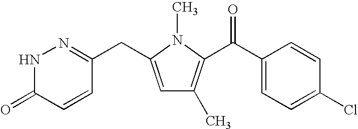 |
| | 6-[[5-(4-chlorobenzoyl)-1,4-dimethyl-1H-pyrrol-2-yl]methyl]- |
| | 3(2H)-pyridazinone or RS 57067 |
| B-3 | 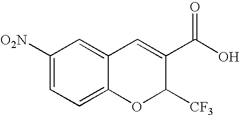 |
| | 6-Nitro-2-trifluoromethyl-2H-1- |
| | benzopyran-3-carboxylic acid |
| B-4 | 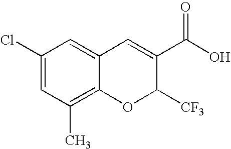 |
| | 6-Chloro-8-methyl-2-trifluoromethyl- |
| | 2H-1-benzopyran-3-carboxylic acid; |
| B-5 | 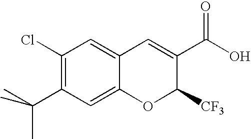 |
| | ((S)-6-Chloro-7-(1,1-dimethylethyl)-2-(trifluoro- |
| | methyl-2H-1-benzopyran-3-carboxylic acid; |
| B-6 | 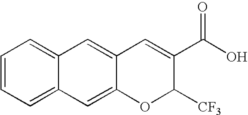 |
| | 2-Trifluoromethyl-2H-naphtho[2,3-b] |
| | pyran-3-carboxylic acid; |
| B-7 | 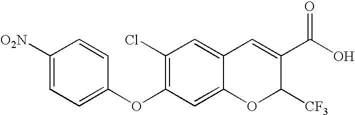 |
| | 6-Chloro-7-(4-nitrophenoxy)-2-(trifluoromethyl)-2H-1- |
| | benzopyran-3-carboxylic acid; |
| B-8 | 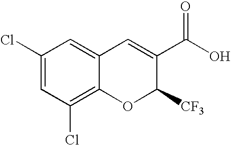 |
| | ((S)-6,8-Dichloro-2-(trifluoromethyl)- |
| | 2H-1-benzopyran-3-carboxylic acid; |
| B-9 | 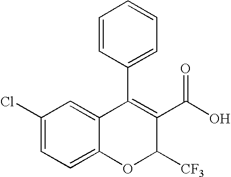 |
| | 6-Chloro-2-(trifluoromethyl)-4-phenyl-2H- |
| | 1-benzopyran-3-carboxylic acid; |
| B-10 | 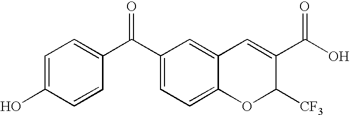 |
| | 6-(4-Hydroxybenzoyl)-2-(trifluoromethyl)- |
| | 2H-1-benzopyran-3-carboxylic acid; |
| B-11 | 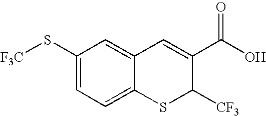 |
| | 2-(Trifluoromethyl)-6-[(trifluoromethyl)thio]- |
| | 2H-1-benzothiopyran-3-carboxylic acid; |
| B-12 | 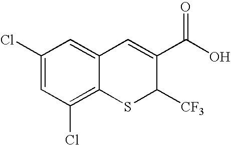 |
| | 6,8-Dichloro-2-trifluoromethyl-2H-1- |
| | benzothiopyran-3-carboxylic acid |
| B-13 | 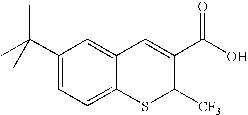 |
| | 6-(1,1-Dimethylethyl)-2-(trifluoromethyl)- |
| | 2H-1-benzothiopyran-3-carboxylic acid; |
| B-14 | 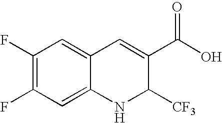 |
| | 6,7-Difluoro-1,2-dihydro-2-(trifluoro |
| | methyl)-3-quinolinecarboxylic acid; |
| B-15 | 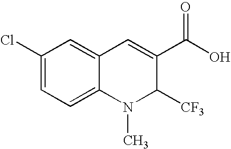 |
| | 6-Chloro-1,2-dihydro-1-methyl-2-(trifluoro |
| | methyl)-3-quinolinecarboxylic acid; |
| B-16 | 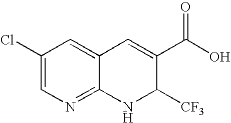 |
| | 6-Chloro-2-(trifluoromethyl)-1,2-dihydro |
| | [1,8]naphthyridine-3-carboxylic acid; |
| B-17 | 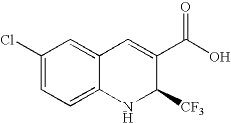 |
| | ((S)-6-Chloro-1,2-dihydro-2-(trifluoro |
| | methyl)-3-quinolinecarboxylic acid |
| B-18 | 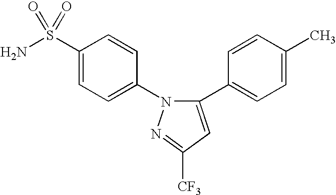 |
| | celecoxib; |
| B-19 | 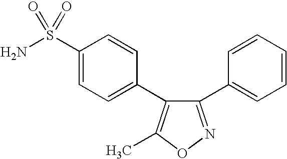 |
| | valdecoxib; |
| B-20 | 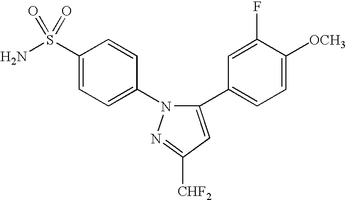 |
| | deracoxib; |
| B-21 | 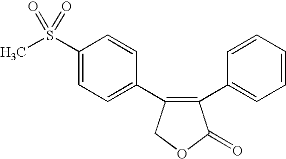 |
| | rofecoxib; |
| B-22 | 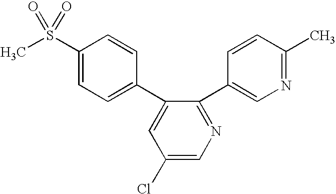 |
| | etoricoxib; |
| B-23 | 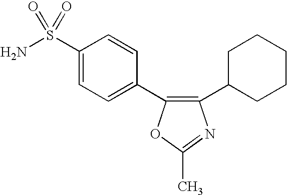 |
| | JTE-522; |
| B-24 | 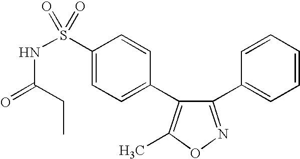 |
| | parecoxib; |
| B-25 | 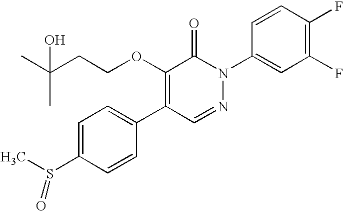 |
| | ABT-963 |
| B-26 | 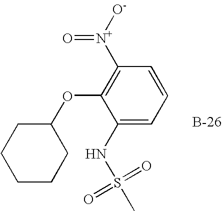 |
| | N-(2-cyclohexyloxynitrophenyl) |
| | methane sulfonamide or NS-398; |
| B-27 | 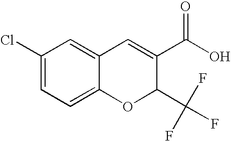 |
| | 6-chloro-2-trifluoromethyl-2H-1-benzopyran-3-carboxylic acid; |
| B-28 | 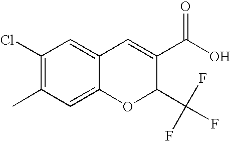 |
| | 6-chloro-7-methyl-2-trifluoromethyl-2H-1- |
| | benzopyran-3-carboxylic acid; |
| B-29 | 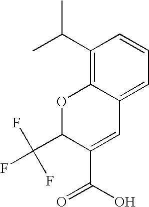 |
| | 8-(1-methylethyl)-2-trifluoromethyl-2H-1- |
| | benzopyran-3-carboxylic acid; |
| B-30 | 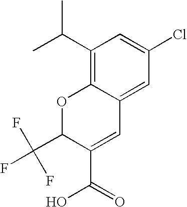 |
| | 6-chloro-8-(1-methylethyl)-2-trifluoromethyl-2H-1- |
| | benzopyran-3-carboxylic acid; |
| B-31 | 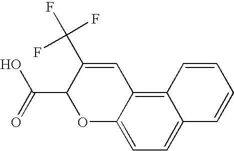 |
| | 2-trifluoromethyl-3H-naphtho[2,1-b]pyran-3-carboxylic acid; |
| B-32 | 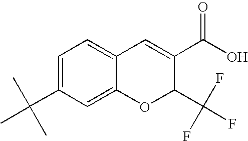 |
| | 7-(1,1-dimethylethyl)-2-trifluoromethyl-2H-1- |
| | benzopyran-3-carboxylic acid; |
| B-33 | 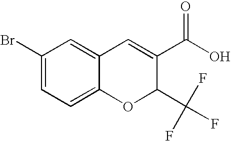 |
| | 6-bromo-2-trifluoromethyl-2H-1-benzopyran-3-carboxylic acid; |
| B-34 | 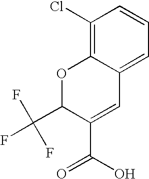 |
| | 8-chloro-2-trifluoromethyl-2H-1-benzopyran-3-carboxylic acid; |
| B-35 | 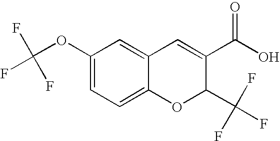 |
| | 6-trifluoromethoxy-2-trifluoromethyl-2H-1- |
| | benzopyran-3-carboxylic acid; |
| B-36 | 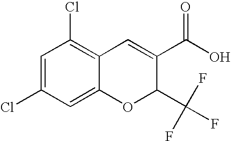 |
| | 5,7-dichloro-2-trifluoromethyl-2H-1- |
| | benzopyran-3-carboxylic acid; |
| B-37 | 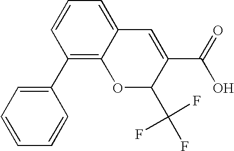 |
| | 8-phenyl-2-trifluoromethyl-2H-1-benzopyran-3-carboxylic acid; |
| B-38 | 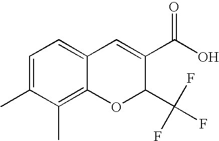 |
| | 7,8-dimethyl-2-trifluoromethyl-2H-1- |
| | benzopyran-3-carboxylic acid; |
| B-39 | 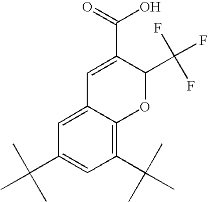 |
| | 6,8-bis(dimethylethyl)-2-trifluoromethyl-2H-1- |
| | benzopyran-3-carboxylic acid; |
| B-40 | 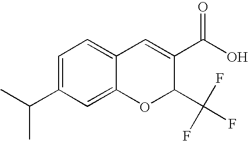 |
| | 7-(1-methylethyl)-2-trifluoromethyl-2H-1- |
| | benzopyran-3-carboxylic acid; |
| B-41 | 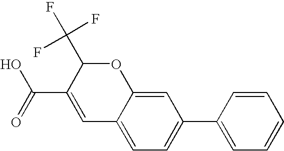 |
| | 7-phenyl-2-trifluoromethyl-2H-1-benzopyran-3-carboxylic acid; |
| B-42 | 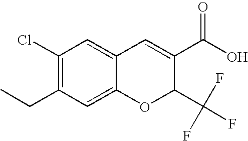 |
| | 6-chloro-7-ethyl-2-trifluoromethyl-2H-1- |
| | benzopyran-3-carboxylic acid; |
| B-43 | 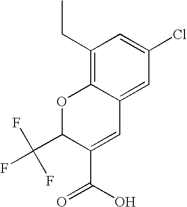 |
| | 6-chloro-8-ethyl-2-trifluoromethyl-2H-1- |
| | benzopyran-3-carboxylic acid; |
| B-44 | 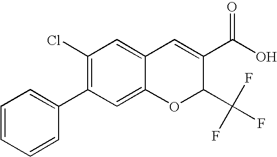 |
| | 6-chloro-7-phenyl-2-trifluoromethyl-2H-1- |
| | benzopyran-3-carboxylic acid; |
| B-45 | 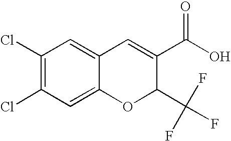 |
| | 6,7-dichloro-2-trifluoromethyl-2H-1- |
| | benzopyran-3-carboxylic acid; |
| B-46 | 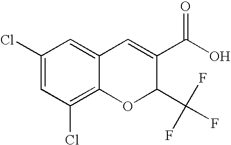 |
| | 6,8-dichloro-2-trifluoromethyl-2H-1- |
| | benzopyran-3-carboxylic acid; |
| B-47 | 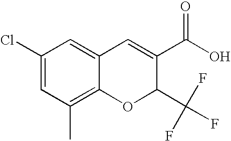 |
| | 6-chloro-8-methyl-2-trifluoromethyl-2H-1- |
| | benzopyran-3-carboxylic acid; |
| B-48 | 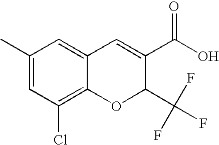 |
| | 8-chloro-6-methyl-2-trifluoromethyl-2H-1- |
| | benzopyran-3-carboxylic acid; |
| B-49 | 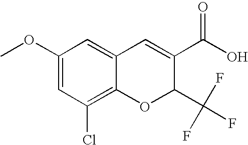 |
| | 8-chloro-6-methoxy-2-trifluoromethyl-2H-1- |
| | benzopyran-3-carboxylic acid; |
| B-50 | 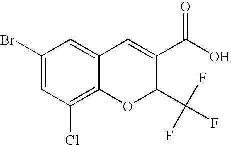 |
| | 6-bromo-8-chloro-2-trifluoromethyl-2H-1- |
| | benzopyran-3-carboxylic acid; |
| B-51 | 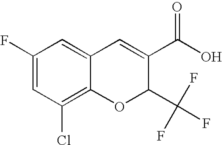 |
| | 8-bromo-6-fluoro-2-trifluoromethyl-2H-1- |
| | benzopyran-3-carboxylic acid; |
| B-52 | 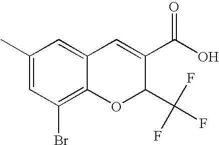 |
| | 8-bromo-6-methyl-2-trifluoromethyl-2H-1- |
| | benzopyran-3-carboxylic acid; |
| B-53 | 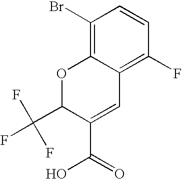 |
| | 8-bromo-5-fluoro-2-trifluoromethyl-2H-1- |
| | benzopyran-3-carboxylic acid; |
| B-54 | 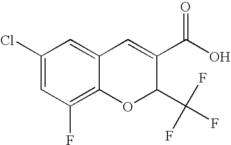 |
| | 6-chloro-8-fluoro-2-trifluoromethyl-2H-1- |
| | benzopyran-3-carboxylic acid; |
| B-55 | 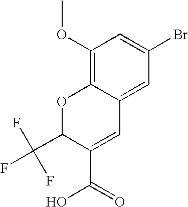 |
| | 6-bromo-8-methoxy-2-trifluoromethyl-2H-1- |
| | benzopyran-3-carboxylic acid; |
| B-56 | 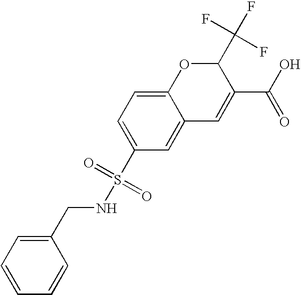 |
| | 6-[[(phenylmethyl)amino]sulfonyl]-2-trifluoromethyl-2H-1- |
| | benzopyran-3-carboxylic acid; |
| B-57 | 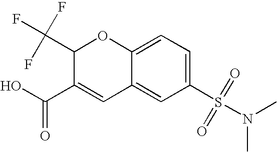 |
| | 6-[(dimethylamino)sulfonyl]-2-trifluoromethyl-2H-1- |
| | benzopyran-3-carboxylic acid; |
| B-58 | 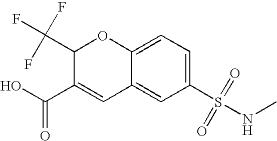 |
| | 6-[(methylamino)sulfonyl]-2-trifluoromethyl-2H-1- |
| | benzopyran-3-carboxylic acid; |
| B-59 | 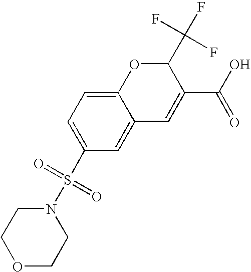 |
| | 6-[(4-morpholino)sulfonyl]-2-trifluoromethyl-2H-1- |
| | benzopyran-3-carboxylic acid; |
| B-60 | 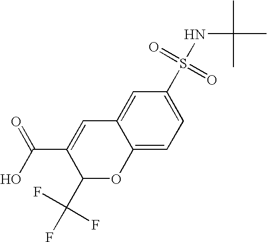 |
| | 6-[(1,1-dimethylethyl)aminosulfonyl]-2-trifluoromethyl-2H-1- |
| | benzopyran-3-carboxylic acid; |
| B-61 | 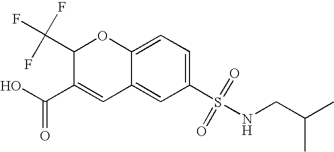 |
| | 6-[(2-methylpropyl)aminosulfonyl]-2-trifluoromethyl-2H-1- |
| | benzopyran-3-carboxylic acid; |
| B-62 | 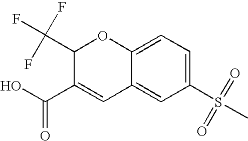 |
| | 6-methylsulfonyl-2-trifluoromethyl-2H-1- |
| | benzopyran-3-carboxylic acid; |
| B-63 | 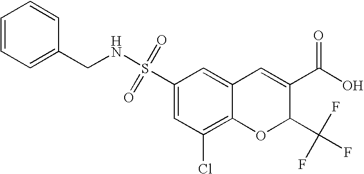 |
| | 8-chloro-6-[[(phenylmethyl)amino]sulfonyl]-2- |
| | trifluoromethyl-2H-1-benzopyran-3-carboxylic acid; |
| B-64 | 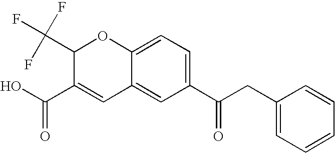 |
| | 6-phenylacetyl-2-trifluoromethyl-2H-1- |
| | benzopyran-3-carboxylic acid; |
| B-65 | 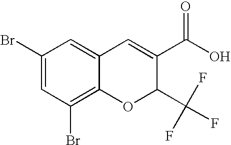 |
| | 6,8-dibromo-2-trifluoromethyl-2H-1- |
| | benzopyran-3-carboxylic acid; |
| B-66 | 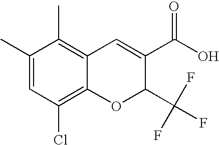 |
| | 8-chloro-5,6-dimethyl-2-trifluoromethyl-2H-1- |
| | benzopyran-3-carboxylic acid; |
| B-67 | 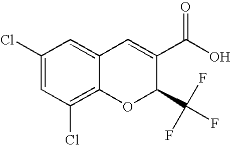 |
| | 6,8-dichloro-(S)-2-trifluoromethyl-2H-1- |
| | benzopyran-3-carboxylic acid; |
| B-68 | 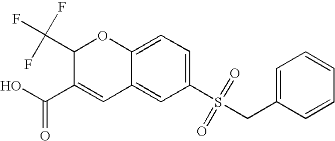 |
| | 6-benzylsulfonyl-2-trifluoromethyl-2H-1- |
| | benzopyran-3-carboxylic acid; |
| B-69 | 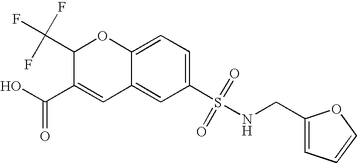 |
| | 6-[[N-(2-furylmethyl)amino]sulfonyl]-2-trifluoromethyl-2H-1- |
| | benzopyran-3-carboxylic acid; |
| B-70 | 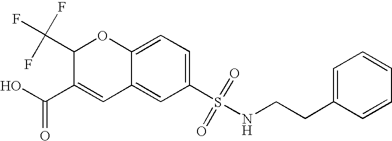 |
| | 6-[[N-(2-phenylethyl)amino]sulfonyl]-2-trifluoromethyl-2H-1- |
| | benzopyran-3-carboxylic acid; |
| B-71 | 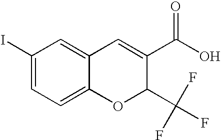 |
| | 6-iodo-2-trifluoromethyl-2H-1-benzopyran-3-carboxylic acid; |
| B-72 | 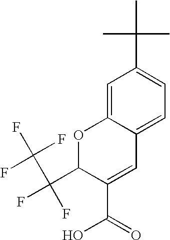 |
| | 7-(1,1-dimethylethyl)-2-pentafluoroethyl-2H-1- |
| | benzopyran-3-carboxylic acid; |
| B-73 | 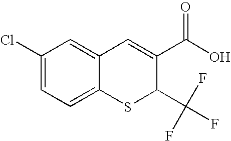 |
| | 6-chloro-2-trifluoromethyl-2H-1- |
| | benzothiopyran-3-carboxylic acid; |
| B-74 | 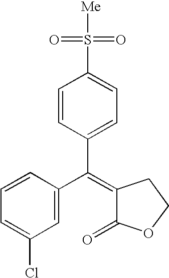 |
| | 3-[(3-Chloro-phenyl)-(4-methanesulfonyl-phenyl)- |
| | methylene]-dihydro-furan-2-one or BMS-347070; |
| B-75 | 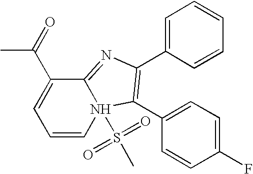 |
| | 8-acetyl-3-(4-fluorophenyl)-2-(4-methylsulfonyl)phenyl- |
| | imidazo(1,2-a)pyridine; |
| B-76 | 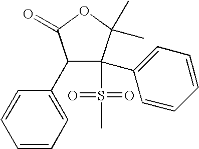 |
| | 5,5-dimethyl-4-(4-methylsulfonyl)phenyl-3- |
| | phenyl-2-(5H)-furanone; |
| B-77 | 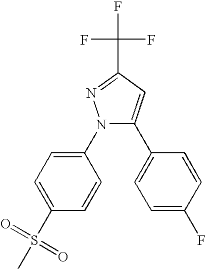 |
| | 5-(4-fluorophenyl)-1-[4-(methylsulfonyl)phenyl]-3- |
| | (trifluoromethyl)pyrazole; |
| B-78 | 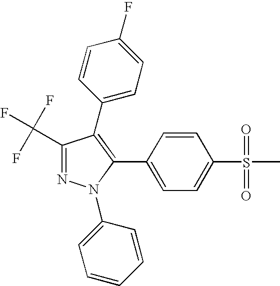 |
| | 4-(4-fluorophenyl)-5-[4-(methylsulfonyl)phenyl]-1-phenyl-3- |
| | (trifluoromethyl)pyrazole; |
| B-79 | 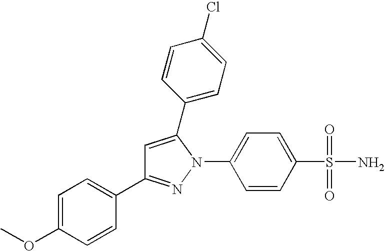 |
| | 4-(5-(4-chlorophenyl)-3-(4-methoxyphenyl)-1H- |
| | pyrazol-1-yl)benzenesulfonamide; |
| B-80 | 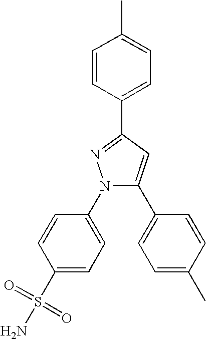 |
| | 4-(3,5-bis(4-methylphenyl)-1H-pyrazol-1- |
| | yl)benzenesulfonamide; |
| B-81 | 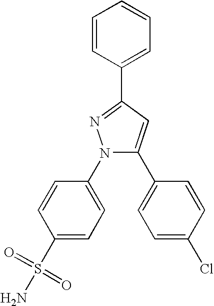 |
| | 4-(5-(4-chlorophenyl)-3-phenyl-1H-pyrazol-1- |
| | yl)benzenesulfonamide; |
| B-82 | 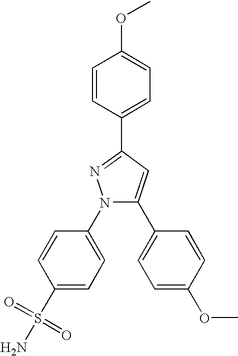 |
| | 4-(3,5-bis(4-methoxyphenyl)-1H-pyrazol-1- |
| | yl)benzenesulfonamide; |
| B-83 | 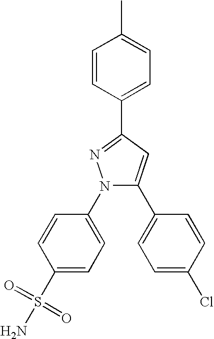 |
| | 4-(5-(4-chlorophenyl)-3-(4-methylphenyl)-1H-pyrazol-1- |
| | yl)benzenesulfonamide; |
| B-84 | 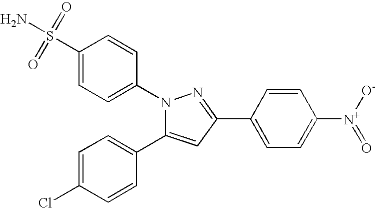 |
| | 4-(5-(4-chlorophenyl)-3-(4-nitrophenyl)-1H- |
| | pyrazol-1-yl)benzenesulfonamide; |
| B-85 | 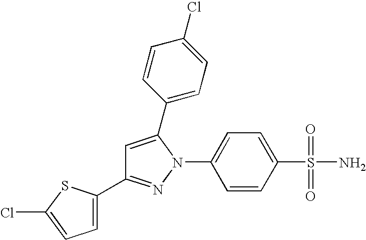 |
| | 4-(5-(4-chlorophenyl)-3-(5-chloro-2-thienyl)-1H- |
| | pyrazol-1-yl)benzenesulfonamide; |
| B-86 | 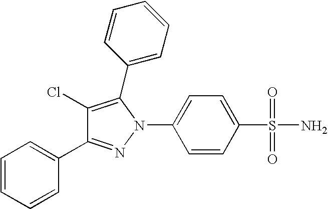 |
| | 4-(4-chloro-3,5-diphenyl-1H-pyrazol-1-yl)benzenesulfonamide; |
| B-87 | 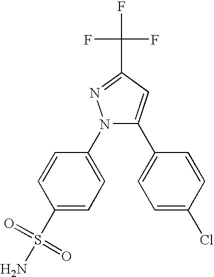 |
| | 4-[5-(4-chlorophenyl)-3-(trifluoromethyl)-1H- |
| | pyrazol-1-yl]benzenesulfonamide; |
| B-88 | 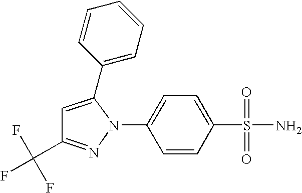 |
| | 4-[5-phenyl-3-(trifluoromethyl)-1H-pyrazol-1- |
| | yl]benzenesulfonamide; |
| B-89 | 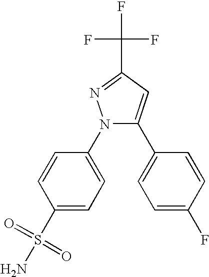 |
| | 4-[5-(4-fluorophenyl)-3-(trifluoromethyl)-1H- |
| | pyrazol-1-yl]benzenesulfonamide; |
| B-90 | 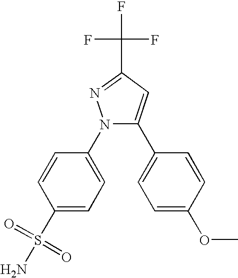 |
| | 4-[5-(4-methoxyphenyl)-3-(trifluoromethyl)-1H- |
| | pyrazol-1-yl]benzenesulfonamide; |
| B-91 | 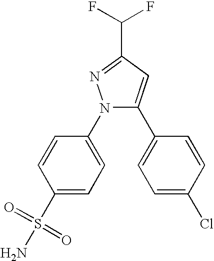 |
| | 4-[5-(4-chlorophenyl)-3-(difluoromethyl)-1H-pyrazol- |
| | 1-yl]benzenesulfonamide; |
| B-92 | 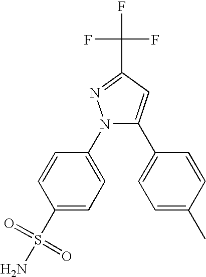 |
| | 4-[5-(4-methylphenyl)-3-(trifluoromethyl)-1H- |
| | pyrazol-1-yl]benzenesulfonamide; |
| B-93 | 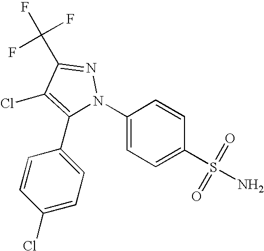 |
| | 4-[4-chloro-5-(4-chlorophenyl)-3-(trifluoromethyl)-1H- |
| | pyrazol-1-yl]benzenesulfonamide; |
| B-94 | 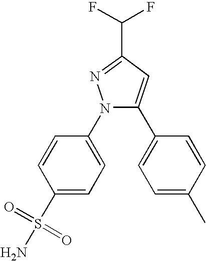 |
| | 4-[3-(difluoromethyl)-5-(4-methylphenyl)-1H-pyrazol- |
| | 1-yl]benzenesulfonamide; |
| B-95 | 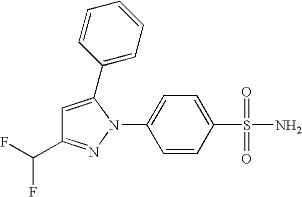 |
| | 4-[3-(difluoromethyl)-5-phenyl-1H-pyrazol- |
| | 1-yl]benzenesulfonamide; |
| B-96 | 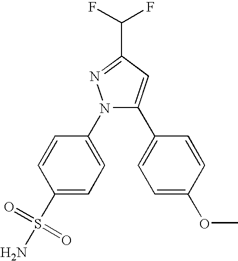 |
| | 4-[3-(difluoromethyl)-5-(4-methoxyphenyl)-1H- |
| | pyrazol-1-yl]benzenesulfonamide; |
| B-97 | 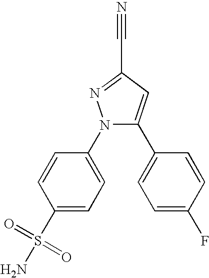 |
| | 4-[3-cyano-5-(4-fluorophenyl)-1H-pyrazol-1- |
| | yl]benzenesulfonamide; |
| B-98 | 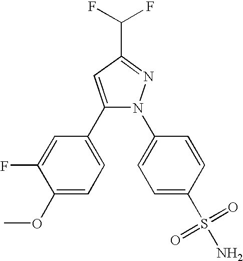 |
| | 4-[3-(difluoromethyl)-5-(3-fluoro-4-methoxyphenyl)-1H- |
| | pyrazol-1-yl]benzenesulfonamide; |
| B-99 | 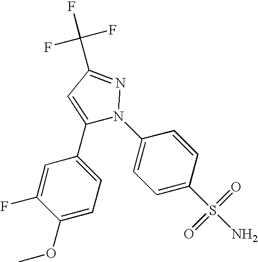 |
| | 4-{5-(3-fluoro-4-methoxyphenyl)-3-(trifluoromethyl)- |
| | 1H-pyrazol-1-yl]benzenesulfonamide; |
| B-100 | 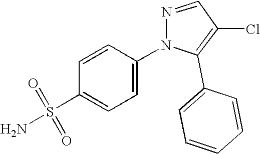 |
| | 4-[4-chloro-5-phenyl-1H-pyrazol-1-yl]benzenesulfonamide; |
| B-101 | 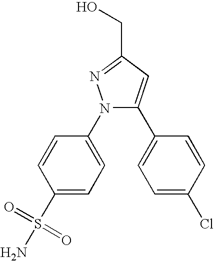 |
| | 4-[5-(4-chlorophenyl)-3-(hydroxymethyl)-1H-pyrazol-1- |
| | yl]benzenesulfonamide; |
| B-102 | 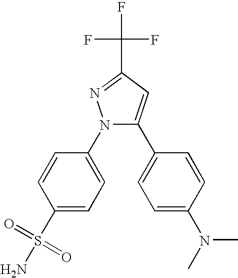 |
| | 4-[5-(4-(N,N-dimethylamino)phenyl)-3-(trifluoromethyl)- |
| | 1H-pyrazol-1-yl]benzenesulfonamide; |
| B-103 | 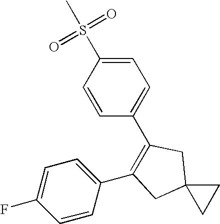 |
| | 5-(4-fluorophenyl)-6-[4- |
| | (methylsulfonyl)phenyl]spiro[2.4]hept-5-ene; |
| B-104 | 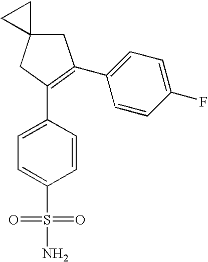 |
| | 4-[6-(4-fluorophenyl)spiro[2.4]hept-5-en-5- |
| | yl]benzenesulfonamide; |
| B-105 | 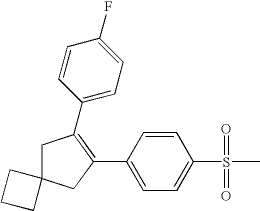 |
| | 6-(4-fluorophenyl)-7-[4-(methylsulfonyl)phenyl]- |
| | spiro[3.4]oct-6-ene; |
| B-106 | 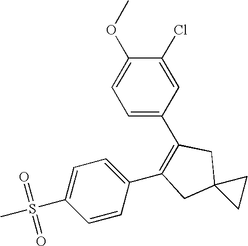 |
| | 5-(3-chloro-4-methoxyphenyl)-6-[4-(methylsulfonyl)phenyl]- |
| | spiro[2.4]hept-5-ene; |
| B-107 | 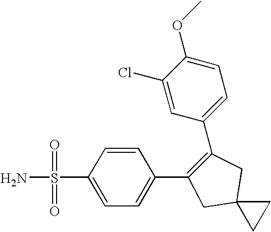 |
| | 4-[6-(3-chloro-4-methoxyphenyl)spiro[2.4]hept- |
| | 5-en-5-yl]benzenesulfonamide; |
| B-108 | 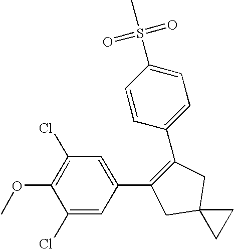 |
| | 5-(3,5-dichloro-4-methoxyphenyl)-6-[4-(methylsulfonyl)- |
| | phenyl]spiro[2.4]hept-5-ene; |
| B-109 | 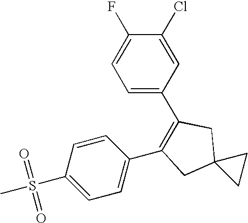 |
| | 5-(3-chloro-4-fluorophenyl)-6-[4-(methylsulfonyl)phenyl]- |
| | spiro[2.4]hept-5-ene; |
| B-110 | 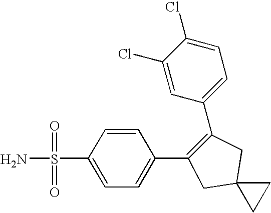 |
| | 4-[6-(3,4-dichlorophenyl)spiro[2.4]hept-5-en-5- |
| | yl]benzenesulfonamide; |
| B-111 | 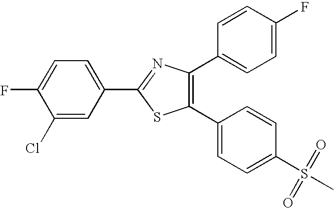 |
| | 2-(3-chloro-4-fluorophenyl)-4-(4-fluorophenyl)-5- |
| | (4-methylsulfonylphenyl)thiazole; |
| B-112 | 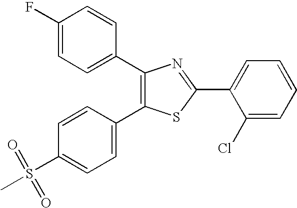 |
| | 2-(2-chlorophenyl)-4-(4-fluorophenyl)-5- |
| | (4-methylsulfonylphenyl)thiazole; |
| B-113 | 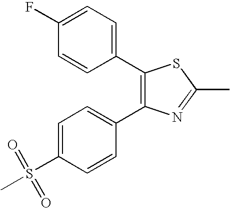 |
| | 5-(4-fluorophenyl)-4-(4-methylsulfonylphenyl)-2-methylthiazole; |
| B-114 | 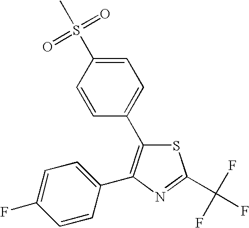 |
| | 4-(4-fluorophenyl)-5-(4-methylsulfonylphenyl)-2- |
| | trifluoromethylthiazole; |
| B-115 | 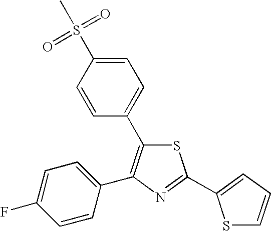 |
| | 4-(4-fluorophenyl)-5-(4-methylsulfonylphenyl)-2- |
| | (2-thienyl)thiazole; |
| B-116 | 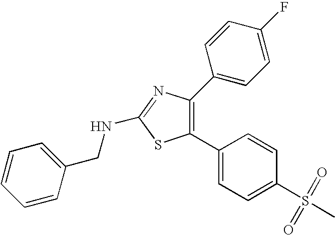 |
| | 4-(4-fluorophenyl)-5-(4-methylsulfonylphenyl)-2- |
| | benzylaminothiazole; |
| B-117 | 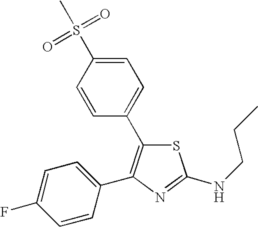 |
| | 4-(4-fluorophenyl)-5-(4-methylsulfonylphenyl)-2- |
| | (1-propylamino)thiazole; |
| B-118 | 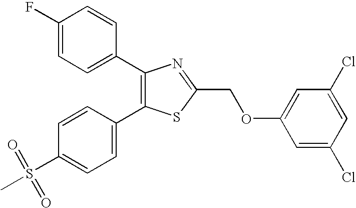 |
| | 2-((3,5-dichlorophenoxy)methyl)-4-(4-fluorophenyl)-5- |
| | [4-(methylsulfonyl)phenyl]thiazole; |
| B-119 | 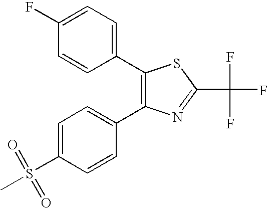 |
| | 5-(4-fluorophenyl)-4-(4-methylsulfonylphenyl)-2- |
| | trifluoromethylthiazole; |
| B-120 | 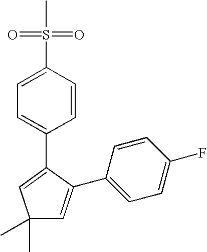 |
| | 1-methylsulfonyl-4-[1,1-dimethyl-4-(4-fluorophenyl)- |
| | cyclopenta-2,4-dien-3-yl]benzene; |
| B-121 | 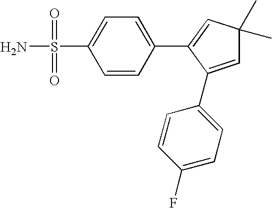 |
| | 4-[4-(4-fluorophenyl)-1,1-dimethylcyclopenta-2,4- |
| | dien-3-yl]benzenesulfonamide; |
| B-122 | 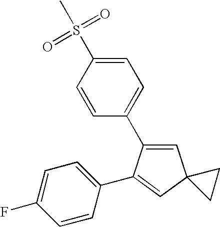 |
| | 5-(4-fluorophenyl)-6-[4-(methylsulfonyl)phenyl]- |
| | spiro[2.4]hepta-4,6-diene; |
| B-123 | 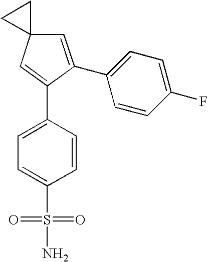 |
| | 4-[6-(4-fluorophenyl)spiro[2.4]hepta-4,6-dien-5- |
| | yl]benzenesulfonamide; |
| B-124 | 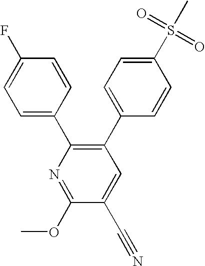 |
| | 6-(4-fluorophenyl)-2-methoxy-5-[4-(methylsulfonyl)- |
| | phenyl]-pyridine-3-carbonitrile; |
| B-125 | 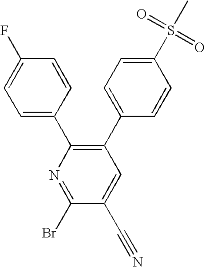 |
| | 2-bromo-6-(4-fluorophenyl)-5-[4-(methylsulfonyl)phenyl]- |
| | pyridine-3-carbonitrile; |
| B-126 | 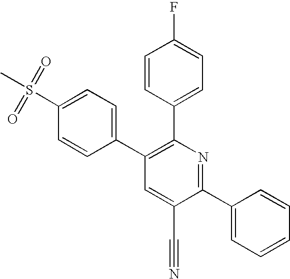 |
| | 6-(4-fluorophenyl)-5-[4-(methylsulfonyl)phenyl]- |
| | 2-phenyl-pyridine-3-carbonitrile; |
| B-127 | 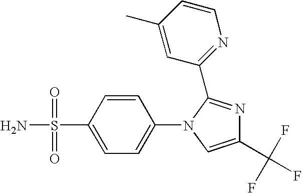 |
| | 4-[2-(4-methylpyridin-2-yl)-4-(trifluoromethyl)-1H- |
| | imidazol-1-yl]benzenesulfonamide; |
| B-128 | 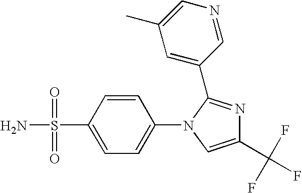 |
| | 4-[2-(5-methylpyridin-3-yl)-4-(trifluoromethyl)-1H- |
| | imidazol-1-yl]benzenesulfonamide; |
| B-129 | 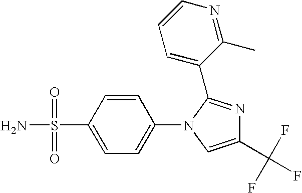 |
| | 4-[2-(2-methylpyridin-3-yl)-4-(trifluoromethyl)-1H- |
| | imidazol-1-yl]benzenesulfonamide; |
| B-130 | 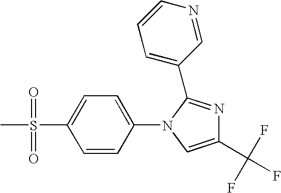 |
| | 3-[1-[4-(methylsulfonyl)phenyl]-4-(trifluoromethyl)- |
| | 1H-imidazol-2-yl]pyridine; |
| B-131 | 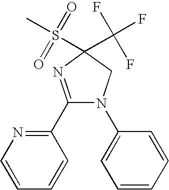 |
| | 2-[1-[4-(methylsulfonyl)phenyl-4-(trifluoromethyl)]- |
| | 1H-imidazol-2-yl]pyridine; |
| B-132 | 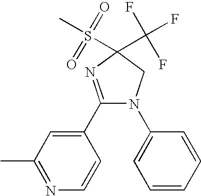 |
| | 2-methyl-4-[1-[4-(methylsulfonyl)phenyl-4-(trifluoromethyl)]- |
| | 1H-imidazol-2-yl]pyridine; |
| B-133 | 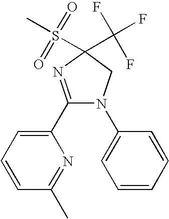 |
| | 2-methyl-6-[1-[4-(methylsulfonyl)phenyl-4-(trifluoromethyl)]- |
| | 1H-imidazol-2-yl]pyridine; |
| B-134 | 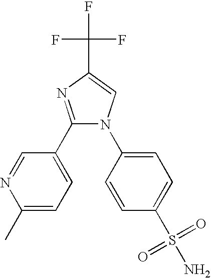 |
| | 4-[2-(6-methylpyridin-3-yl)-4-(trifluoromethyl)-1H-imidazol- |
| | 1-yl]benzenesulfonamide; |
| B-135 | 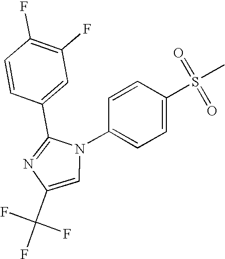 |
| | 2-(3,4-difluorophenyl)-1-[4-(methylsulfonyl)phenyl]- |
| | 4-(trifluoromethyl)-1H-imidazole; |
| B-136 | 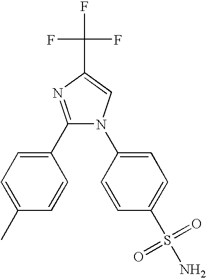 |
| | 4-[2-(4-methylphenyl)-4-(trifluoromethyl)-1H-imidazol-1- |
| | yl]benzenesulfonamide; |
| B-137 | 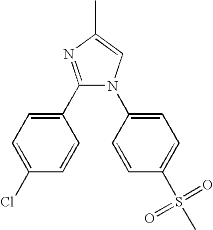 |
| | 2-(4-chlorophenyl)-1-[4-(methylsulfonyl)phenyl]- |
| | 4-methyl-1H-imidazole; |
| B-138 | 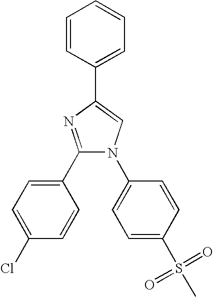 |
| | 2-(4-chlorophenyl)-1-[4-(methylsulfonyl)phenyl]-4- |
| | phenyl-1H-imidazole; |
| B-139 | 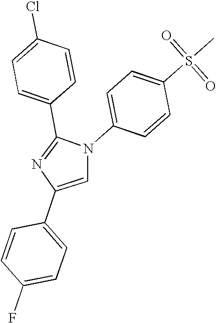 |
| | 2-(4-chlorophenyl)-4-(4-fluorophenyl)-1-[4- |
| | (methylsulfonyl)phenyl]-1H-imidazole; |
| B-140 | 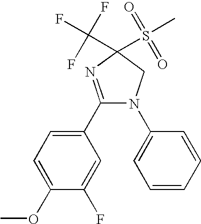 |
| | 2-(3-fluoro-4-methoxyphenyl)-1-[4-(methylsulfonyl)phenyl- |
| | 4-(trifluoromethyl)]-1H-imidazole; |
| B-141 | 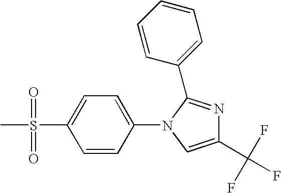 |
| | 1-[4-(methylsulfonyl)phenyl]-2-phenyl-4-trifluoromethyl- |
| | 1H-imidazole; |
| B-142 | 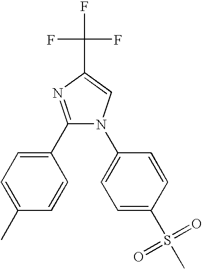 |
| | 2-(4-methylphenyl)-1-[4-(methylsulfonyl)phenyl]-4- |
| | trifluoromethyl-1H-imidazole; |
| B-143 | 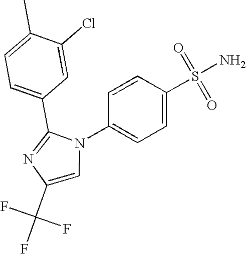 |
| | 4-[2-(3-chloro-4-methylphenyl)-4-(trifluoromethyl)-1H- |
| | imidazol-1-yl]benzenesulfonamide; |
| B-144 | 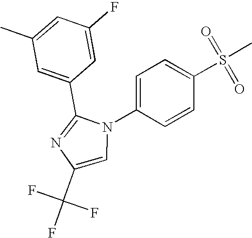 |
| | 2-(3-fluoro-5-methylphenyl)-1-[4-(methylsulfonyl)phenyl]-4- |
| | (trifluoromethyl)-1H-imidazole; |
| B-145 | 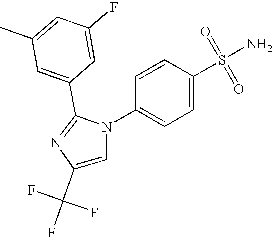 |
| | 4-[2-(3-fluoro-5-methylphenyl)-4-(trifluoromethyl)-1H- |
| | imidazol-1-yl]benzenesulfonamide; |
| B-146 | 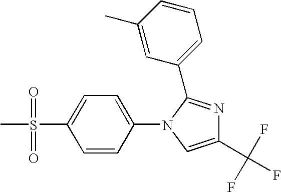 |
| | 2-(3-methylphenyl)-1-[4-(methylsulfonyl)phenyl]-4- |
| | trifluoromethyl-1H-imidazole; |
| B-147 | 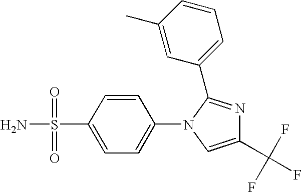 |
| | 4-[2-(3-methylphenyl)-4-trifluoromethyl-1H-imidazol-1- |
| | yl]benzenesulfonamide; |
| B-148 | 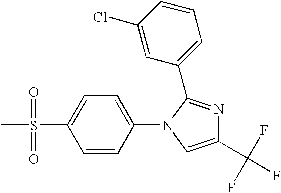 |
| | 1-[4-(methylsulfonyl)phenyl]-2-(3-chlorophenyl)-4- |
| | trifluoromethyl-1H-imidazole; |
| B-149 | 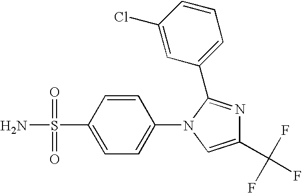 |
| | 4-[2-(3-chlorophenyl)-4-trifluoromethyl-1H-imidazol-1- |
| | yl]benzenesulfonamide; |
| B-150 | 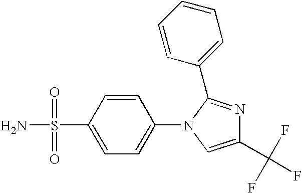 |
| | 4-[2-phenyl-4-trifluoromethyl-1H-imidazol-1- |
| | yl]benzenesulfonamide; |
| B-151 | 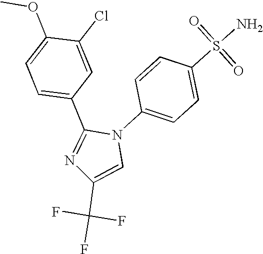 |
| | 4-[2-(4-methoxy-3-chlorophenyl)-4-trifluoromethyl-1H- |
| | imidazol-1-yl]benzenesulfonamide; |
| B-152 | 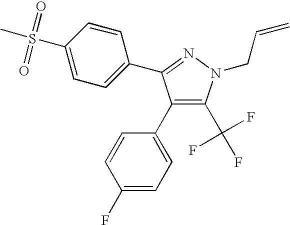 |
| | 1-allyl-4-(4-fluorophenyl)-3-[4-(methylsulfonyl)phenyl]-5- |
| | (trifluoromethyl)-1H-pyrazole; |
| B-153 | 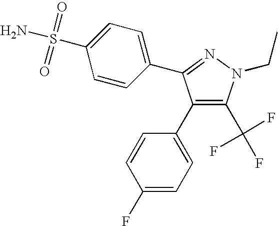 |
| | 4-[1-ethyl-4-(4-fluorophenyl)-5-(trifluoromethyl)-1H-pyrazol- |
| | 3-yl]benzenesulfonamide; |
| B-154 | 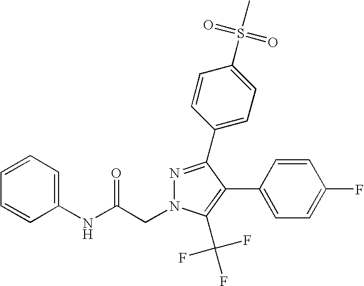 |
| | N-phenyl-[4-(4-fluorophenyl)-3-[4-(methylsulfonyl)phenyl]-5- |
| | (trifluoromethyl)-1H-pyrazol-1-yl]acetamide; |
| B-155 | 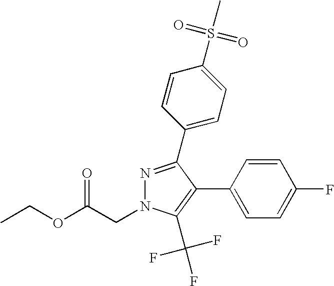 |
| | ethyl [4-(4-fluorophenyl)-3-[4-(methylsulfonyl)phenyl]-5- |
| | (trifluoromethyl)-1H-pyrazol-1-yl]acetate; |
| B-156 | 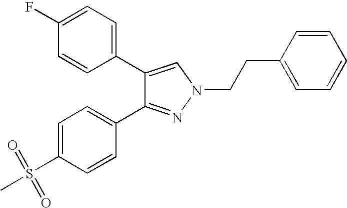 |
| | 4-(4-fluorophenyl)-3-[4-(methylsulfonyl)phenyl]-1- |
| | (2-phenylethyl)-1H-pyrazole; |
| B-157 | 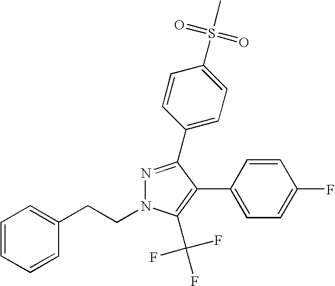 |
| | 4-(4-fluorophenyl)-3-[4-(methylsulfonyl)phenyl]-1-(2- |
| | phenylethyl)-5-(trifluoromethyl)pyrazole; |
| B-158 | 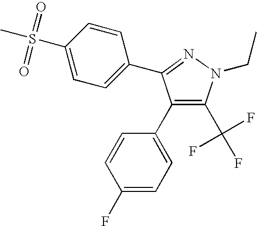 |
| | 1-ethyl-4-(4-fluorophenyl)-3-[4-(methylsulfonyl)phenyl]-5- |
| | (trifluoromethyl)-1H-pyrazole; |
| B-159 | 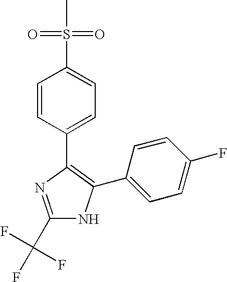 |
| | 5-(4-fluorophenyl)-4-(4-methylsulfonylphenyl)-2- |
| | trifluoromethyl-1H-imidazole; |
| B-160 | 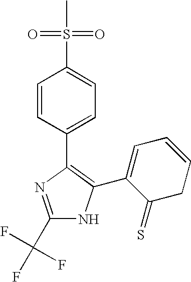 |
| | 4-[4-(methylsulfonyl)phenyl]-5-(2-thiophenyl)-2- |
| | (trifluoromethyl)-1H-imidazole; |
| B-161 | 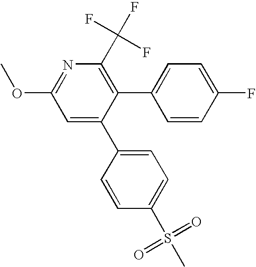 |
| | 5-(4-fluorophenyl)-2-methoxy-4-[4-(methylsulfonyl)phenyl]- |
| | 6-(trifluoromethyl)pyridine; |
| B-162 | 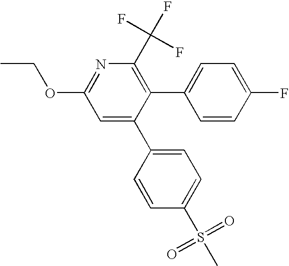 |
| | 2-ethoxy-5-(4-fluorophenyl)-4-[4-(methylsulfonyl)phenyl]-6- |
| | (trifluoromethyl)pyridine |
| B-163 | 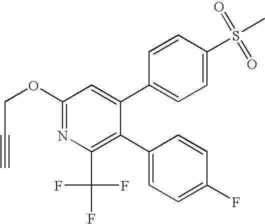 |
| | 5-(4-fluorophenyl)-4-[4-(methylsulfonyl)phenyl]-2- |
| | (2-propynyloxy)-6-(trifluoromethyl)pyridine; |
| B-164 | 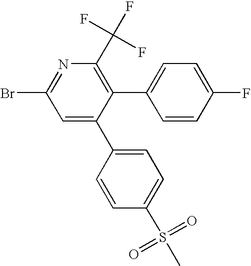 |
| | 2-bromo-5-(4-fluorophenyl)-4-[4-(methylsulfonyl)phenyl]-6- |
| | (trifluoromethyl)pyridine; |
| B-165 | 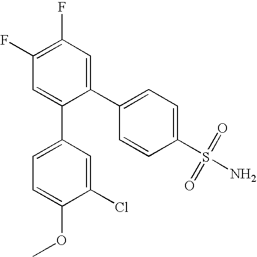 |
| | 4-[2-(3-chloro-4-methoxyphenyl)-4,5- |
| | difluorophenyl]benzenesulfonamide; |
| B-166 | 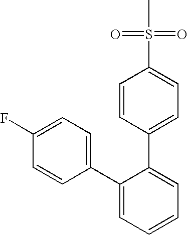 |
| | 1-(4-fluorophenyl)-2-[4-(methylsulfonyl)phenyl]benzene; |
| B-167 | 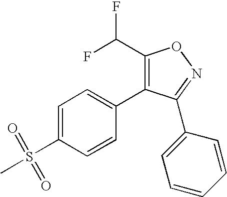 |
| | 5-difluoromethyl-4-(4-methylsulfonylphenyl)-3- |
| | phenylisoxazole; |
| B-168 | 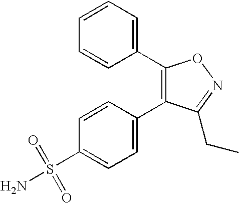 |
| | 4-[3-ethyl-5-phenylisoxazol-4-yl]benzenesulfonamide; |
| B-169 | 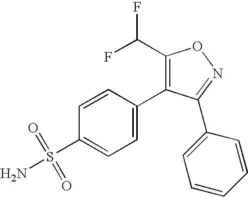 |
| | 4-[5-difluoromethyl-3-phenylisoxazol-4-yl]benzenesulfonamide; |
| B-170 | 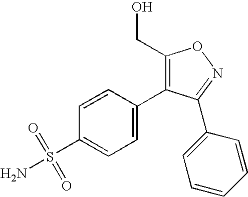 |
| | 4-[5-hydroxymethyl-3-phenylisoxazol-4-yl]benzenesulfonamide; |
| B-171 | 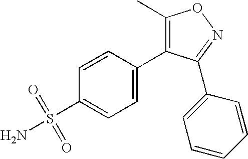 |
| | 4-[5-methyl-3-phenyl-isoxazol-4-yl]benzenesulfonamide; |
| B-172 | 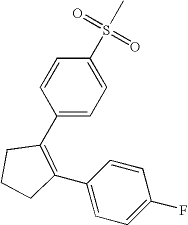 |
| | 1-[2-(4-fluorophenyl)cyclopenten-1-yl]-4- |
| | (methylsulfonyl)benzene; |
| B-173 | 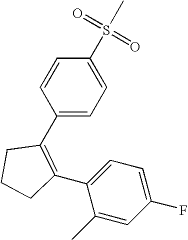 |
| | 1-[2-(4-fluoro-2-methylphenyl)cyclopenten-1-yl]-4- |
| | (methylsulfonyl)benzene; |
| B-174 | 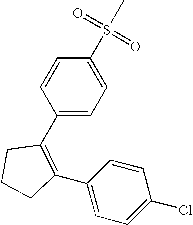 |
| | 1-[2-(4-chlorophenyl)cyclopenten-1-yl]-4- |
| | (methylsulfonyl)benzene; |
| B-175 | 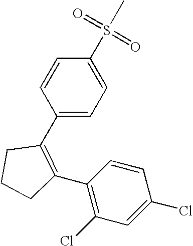 |
| | 1-[2-(2,4-dichlorophenyl)cyclopenten-1-yl]-4- |
| | (methylsulfonyl)benzene; |
| B-176 | 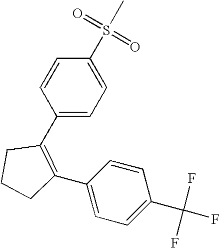 |
| | 1-[2-(4-trifluoromethylphenyl)cyclopenten-1-yl]-4- |
| | (methylsulfonyl)benzene; |
| B-177 | 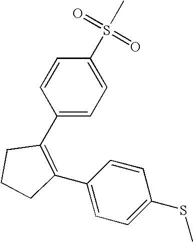 |
| | 1-[2-(4-methylthiophenyl)cyclopenten-1-yl]-4- |
| | (methylsulfonyl)benzene; |
| B-178 | 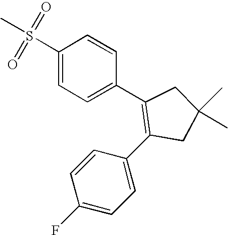 |
| | 1-[2-(4-fluorophenyl)-4,4-dimethylcyclopenten-1-yl]-4- |
| | (methylsulfonyl)benzene; |
| B-179 | 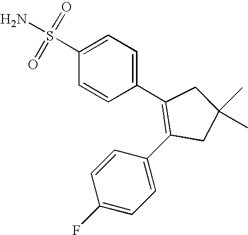 |
| | 4-[2-(4-fluorophenyl)-4,4-dimethylcyclopenten-1- |
| | yl]benzenesulfonamide; |
| B-180 | 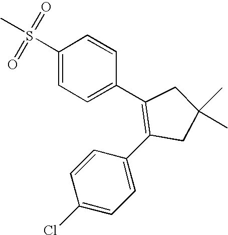 |
| | 1-[2-(4-chlorophenyl)-4,4-dimethylcyclopenten-1-yl]-4- |
| | (methylsulfonyl)benzene; |
| B-181 | 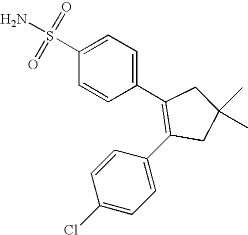 |
| | 4-[2-(4-chlorophenyl)-4,4-dimethylcyclopenten-1- |
| | yl]benzenesulfonamide |
| B-182 | 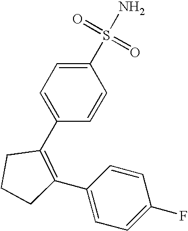 |
| | 4-[2-(4-fluorophenyl)cyclopenten-1-yl]benzenesulfonamide; |
| B-183 | 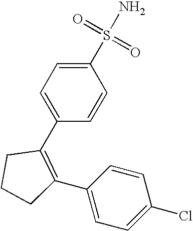 |
| | 4-[2-(4-chlorophenyl)cyclopenten-1-yl]benzenesulfonamide; |
| B-184 | 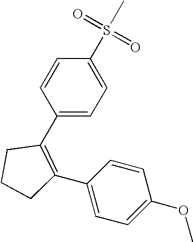 |
| | 1-[2-(4-methoxyphenyl)cyclopenten-1-yl]-4- |
| | (methylsulfonyl)benzene; |
| B-185 | 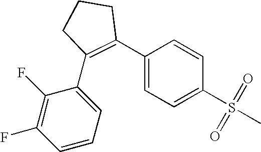 |
| | 1-[2-(2,3-difluorophenyl)cyclopenten-1-yl]-4- |
| | (methylsulfonyl)benzene; |
| B-186 | 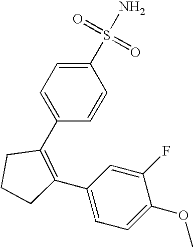 |
| | 4-[2-(3-fluoro-4-methoxyphenyl)cyclopenten-1- |
| | yl]benzenesulfonamide; |
| B-187 | 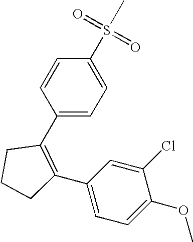 |
| | 1-[2-(3-chloro-4-methoxyphenyl)cyclopenten-1-yl]-4- |
| | (methylsulfonyl)benzene; |
| B-188 | 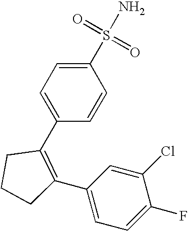 |
| | 4-[2-(3-chloro-4-fluorophenyl)cyclopenten-1- |
| | yl]benzenesulfonamide; |
| B-189 | 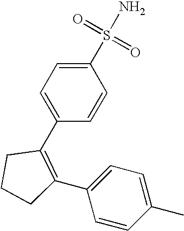 |
| | 4-[2-(2-methylpyridin-5-yl)cyclopenten-1- |
| | yl]benzenesulfonamide; |
| B-190 | 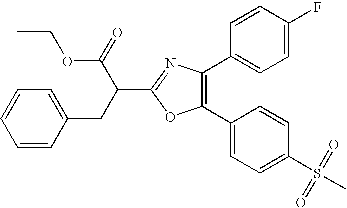 |
| | ethyl 2-[4-(4-fluorophenyl)-5-[4-(methylsulfonyl) phenyl]- |
| | oxazol-2-yl]-2-benzyl-acetate; |
| B-191 | 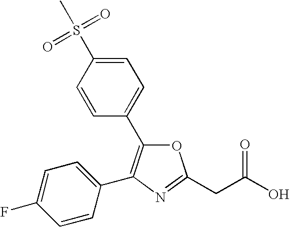 |
| | 2-[4-(4-fluorophenyl)-5-[4-(methylsulfonyl)phenyl]oxazol-2- |
| | yl]acetic acid; |
| B-192 | 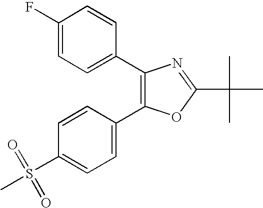 |
| | 2-(tert-butyl)-4-(4-fluorophenyl)-5-[4-(methylsulfonyl)- |
| | phenyl]oxazole; |
| B-193 | 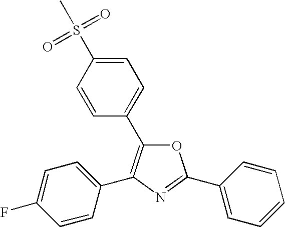 |
| | 4-(4-fluorophenyl)-5-[4-(methylsulfonyl)phenyl]-2- |
| | phenyloxazole; |
| B-194 | 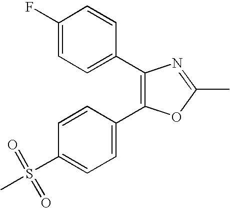 |
| | 4-(4-fluorophenyl)-2-methyl-5-[4- |
| | (methylsulfonyl)phenyl]oxazole |
| B-195 | 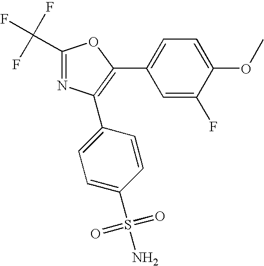 |
| | 4-[5-(3-fluoro-4-methoxyphenyl)-2-trifluoromethyl-4- |
| | oxazolyl]benzenesulfonamide; |
| B-196 | 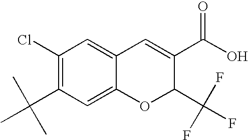 |
| | 6-chloro-7-(1,1-dimethylethyl)-2-trifluoromethyl-2H-1- |
| | benzopyran-3-carboxylic acid; |
| B-197 | 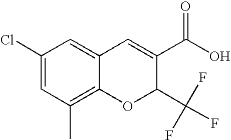 |
| | 6-chloro-8-methyl-2-trifluoromethyl-2H-1-benzopyran-3- |
| | carboxylic acid; |
| B-198 | 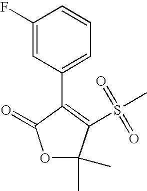 |
| | 5,5-dimethyl-3-(3-fluorophenyl)-4-methylsulfonyl- |
| | 2(5H)-furanone; |
| B-199 | 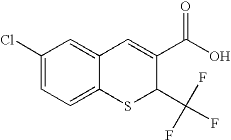 |
| | 6-chloro-2-trifluoromethyl-2H-1-benzothiopyran-3-carboxylic |
| | acid; |
| B-200 | 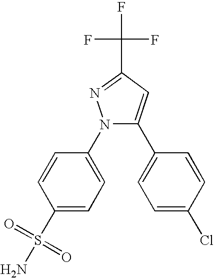 |
| | 4-[5-(4-chlorophenyl)-3-(trifluoromethyl)-1H-pyrazol-1- |
| | yl]benzenesulfonamide; |
| B-201 | 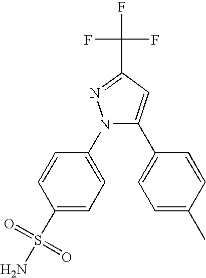 |
| | 4-[5-(4-methylphenyl)-3-(trifluoromethyl)-1H-pyrazol-1- |
| | yl]benzenesulfonamide; |
| B-202 | 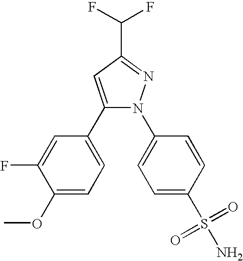 |
| | 4-[5-(3-fluoro-4-methoxyphenyl)-3-(difluoromethyl)-1H- |
| | pyrazol-1-yl]benzenesulfonamide; |
| B-203 | 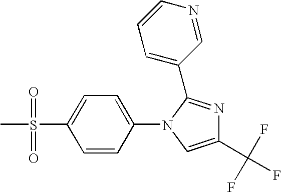 |
| | 3-[1-[4-(methylsulfonyl)phenyl]-4-trifluoromethyl-1H-imidazol- |
| | 2-yl]pyridine; |
| B-204 | 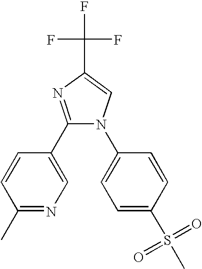 |
| | 2-methyl-5-[1-[4-(methylsulfonyl)phenyl]-4-trifluoromethyl- |
| | 1H-imidazol-2-yl]pyridine; |
| B-205 | 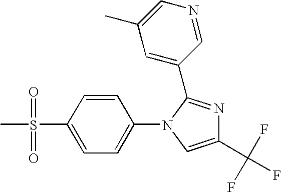 |
| | 4-[2-(5-methylpyridin-3-yl)-4-(trifluoromethyl)-1H-imidazol- |
| | 1-yl]benzenesulfonamide; |
| B-206 | 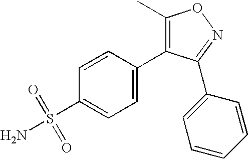 |
| | 4-[5-methyl-3-phenylisoxazol-4-yl]benzenesulfonamide; |
| B-207 | 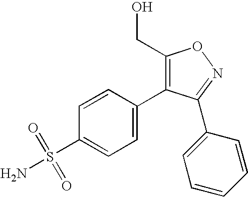 |
| | 4-[5-hydroxymethyl-3-phenylisoxazol-4-yl]benzenesulfonamide; |
| B-208 | 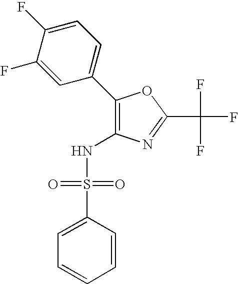 |
| | [2-trifluoromethyl-5-(3,4-difluorophenyl)-4-oxazolyl]- |
| | benzenesulfonamide; |
| B-209 | 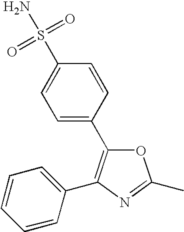 |
| | 4-[2-methyl-4-phenyl-5-oxazolyl]benzenesulfonamide; |
| B-210 | 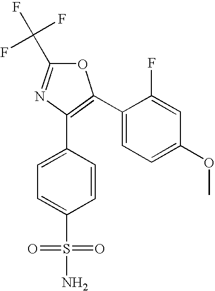 |
| | 4-[5-(2-fluoro-4-methoxyphenyl)-2-trifluoromethyl-4- |
| | oxazolyl]benzenesulfonamide; |
| B-211 | 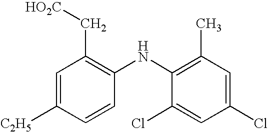 |
| | [2-(2,4-dichloro-6-methyl-phenylamino)-5-ethyl-phenyl]-acetic |
| | acid or COX 189 or Lumiracoxib |
| B-212 | 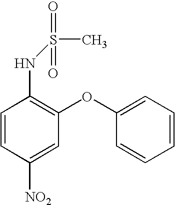 |
| | N-(4-Nitro-2-phenoxy-phenyl)-methanesulfonamide or |
| | Nimesulide |
| B-213 | 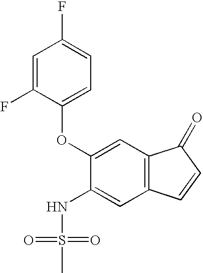 |
| | N-[6-(2,4-Difluoro-phenoxy)-1-oxo-inden-5-yl]- |
| | methanesulfonamide or Flosulide |
| B-214 | 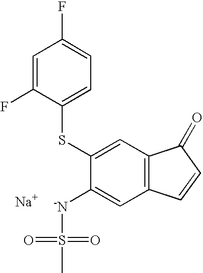 |
| | N-[6-(2,4-Difluoro-phenylsulfanyl)-1-oxo-1H-inden-5-yl]- |
| | methanesulfonamide, soldium salt, or L-745337 |
| B-215 | 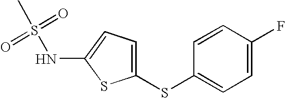 |
| | N-[5-(4-fluoro-phenylsulfanyl)-thiophen-2-yl]- |
| | methanesulfonamide or RWJ-63556 |
| B-216 | 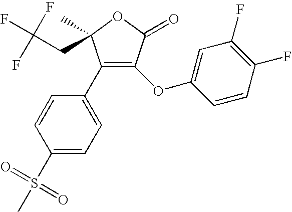 |
| | 3-(3,4-Difluoro-phenoxy)-4-(4-methanesulfonyl-phenyl)-5- |
| | methyl-5-(2,2,2-trifluoro-ethyl)-5H-furan-2-one or L-784512 |
| B-217 | 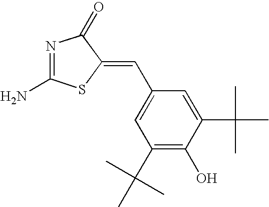 |
| | (5Z)-2-amino-5-[[3,5-bis(1,1-dimethylethyl)-4- |
| | hydroxyphenyl]methylene]-4(5H)-thiolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 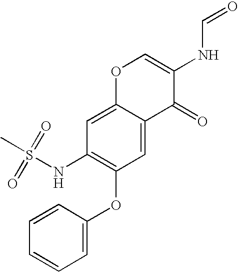 |
| | N-[3-(formylamino)-4-oxo-6-phenoxy-4H-1-benzopyran-7-yl]- |
| | methanesulfonamide or T614 |
| B-225 | D-1367 |
| B-226 | L-748731 |
| B-227 | 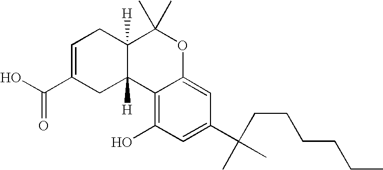 |
| | (6aR, 10aR)-3-(1,1-dimethylheptyl)-6a,7,10,10a-tetrahydro-1- |
| | hydroxy-6,6-dimethyl-6H-dibenzo[b,d]pyran-9-carboxylic |
| | acid or CT 3 |
| B-228 | CGP-28238 |
| B-229 | 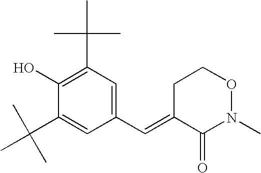 |
| | 4-[[3,5-bis(1,1-dimethylethyl)-4-hydroxyphenyl]methylene]- |
| | dihydro-2-methyl-2H-1,2-oxazin-3(4H)-one or BF-389 |
| B-230 | GR-253035 |
| B-231 | 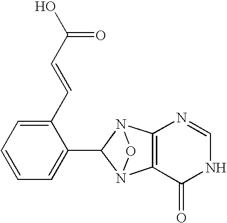 |
| | 2-(6-dioxo-9H-purin-8-yl)cinnamic acid; |
| B-232 | S-2474 |
| B-233 | 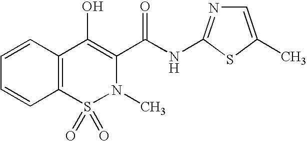 |
| | meloxicam |

According to an embodiment of the present invention, second drugs (see Table 2) are administered in combination with the COX 2 inhibitors of Tables 1 and 1A. Also see Tables 2A-2B below. Examples of second drugs within the categories described herein include, but are not limited to, those given in Table 2 below:

TABLE 2

| | Second | |
|---|---|---|
| Category | Drug | Name |
| Glutamate Antagonists | C-1 | riluzole (Rilutek ®) |
| | C-2 | Lamotrigine |
| | C-3 | dextromethorphan |
| | C-4 | EAAT2 |
| | C-5 | Tamoxifen |
| | C-6 | topiramate (Topamax ®) |
| γ-Aminobutyric Acid | C-7 | gabapentin (Neurontin ®) |
| (GABA) Receptor | | |
| Modulators | | |
| N-Methyl-D-Aspartate | C-8 | 2-amino-5-phosphanovalerate (AP5) |
| (NMDA) Receptor | C-9 | Eliprodil |
| Antagonists and | C-10 | 3-((+)-2-carboxypiperazin-4- |
| Desensitizers | | yl) propyl-1-phosphonic acid (CPP) |
| | C-11 | 1-(cis-2-carboxypiperazin-4- |
| | | yl)propyl-1-phosphonic acid (selfotel |
| | | or CGS 19755) |
| | C-12 | D-(E)-4-(3-phosphonopro-2-2nyl) |
| | | piperazine-2-carboxylic acid (CGP |
| | | 37849) |
| | C-13 | DL-(E)-2-amino-4-methyl-5- |
| | | phosphono-3-pentenoic acid ethyl |
| | | ester (CGP 39551) |
| | C-14 | D-(E)-2-amino-4-methyl-5- |
| | | phosphono-3-pentenoic acid (CGP |
| | | 40116) |
| | C-15 | 2R,4R,5S-(2-amino-4,5-(1,2- |
| | | cyclohexyl)-7-phosphonoheptanoic |
| | | acid) (NPC 17742) |
| | C-16 | N-(1-Naphthyl)-N-(3-ethyiphenyl)- |
| | | N-methyl guanidine HCl |
| | | (aptiganel/CNS 1102) |
| | C-17 | (+)-3-methoxy-17-methylmorphinan |
| | | (dextromethorphan) |
| | C-18 | AVP-923 (dextromethorphan and |
| | | enzyme inhibitor) |
| | C-19 | (−)-1-methyl-1-phenyl-1-1,2,3,4- |
| | | tetrahydroisoquinoline HCl (FR |
| | | 115427) |
| | C-20 | (−)-2-(2-chlorophenyl)-2-(methyl- |
| | | amino) cyclohexanone (ketamine) |
| | C-21 | 1-[4-(3-hydroxyphenyl)-1-methyl-4- |
| | | piperidyl]-1-propanone |
| | | (ketobemidone) |
| | C-22 | 3,5-dimethyladamantan-1-amine |
| | | (memantine) |
| | C-23 | 6-dimethylamino-4,4-diphenyl-3- |
| | | heptanone (methadone) |
| | C-24 | 3-(2-carboxypiperizine-4-yl)- |
| | | propenyl-1-phosphonate (dizocilpine |
| | | or MK 801) |
| | C-25 | 1-(1-phenecyclohexyl)piperidine |
| | | (PCP) |
| | C-26 | 1-methyl-4-phenyl-4-piperidine |
| | | carboxylic acid ethyl ester (pethidine |
| | | or meperidine) |
| α-Amino-3-Hydroxy-5- | C-27 | RPR-119990 |
| Methyl-4-Isoxazole | C-28 | LY-300164 or talampanel |
| Proprionate (AMPA) | C-29 | 6-cyano-7-nitorquinoxaline-2,3- |
| Receptor Antagonists | | dione (CNQX) |
| and Desensitizers | C-30 | 6,7-dinitorquinoxaline-2,3-dione |
| | | (DNQX) |
| | C-31 | (3SR, 4aRS, 6RS, 8aRS)-6-[2-(1H- |
| | | tetrazol-5-yl)-ethyl] |
| | | 1,2,3,4,4a,5,6,7,8a- |
| | | decahydroisoquinoline-3-carboxylic |
| | | acid (LY 215490) |
| | C-32 | 6-nitro-7-sulfamoylbenzo(f)- |
| | | quinoxaline-2,3-dione (NNC 079202 |
| | | or NBQX) |
| | C-33 | 1,2,3,6,7,8-hexahydro-3- |
| | | (hydroxyimino)-N,N-dimethyl-7- |
| | | methyl-2-oxobenzo[2,1-b: 3,4-C′] |
| | | dipyrrole-5-solfonaminde (NS 257) |
| | C-34 | 1-(4-aminophenyl)-4 methyl-7,8- |
| | | methylenedeoxy-5H-2,3- |
| | | benzodiazepine (GYKI 52466) |
| | C-35 | 6-chloro-3,4-dihydro-3-(2- |
| | | norbornen-5-yl)-7-sulfanol-1,2,4- |
| | | benzothiadiazine 1,1-dioxide |
| | | (cyclothiazide) |
| | C-36 | 7-chloro-3-methyl-3,4-dihydro-2H- |
| | | 1,2,4-benzothiadiazine s,s-dioxide |
| | | (IDRA 21) |
| Metabotropic | C-37 | (1S, 1′R, 2′R, 3′R)-2-(2,3- |
| Receptor Agonists | | dicarboxycyclospropyl) glycine |
| and Antagonists | | DCG-IV |
| | C-38 | (2S, 1′S, 2′S)-2-(2- |
| | | carboxycyclospropyl) glycine |
| | C-39 | 2-amino-4-phosphonobutyrate (AP4) |
| | C-40 | trans-1-amino-cyclopentyl-1,3- |
| | | dicarboxylate (t-ACPD) |
| | C-41 | serine-O-phosphate (L-SOP) |
| | C-42 | L-(+)-2-amino-3- |
| | | phosphonoproprioionic acid (L-AP3) |
| | C-43 | (S)-4-carboxy-3-hydroxy- |
| | | phenylglycine (S-4C3HPG) |
| | C-44 | (S)-4-carboxyphenylglycine (S- |
| | | 4CPG) |
| | C-45 | (S)-2-amino-2-methyl-4- |
| | | phosphonobutanoic acid (MAP-4) |
| | C-46 | (R, S)-α-methyl-4-carboxy- |
| | | phenylglycine (RS-M4CPG) |
| Glycine Modulators | C-47 | N-(3-[5-chloro-1-(4- |
| | | chlorophenyl)indan-1-yl]propyl)-N- |
| | | methylalanine |
| Branched Chain | C-48 | Valine |
| Amino Acids | C-49 | Leucine |
| | C-50 | Isoleucine |
| Neurotransmitter | C-51 | xaliproden or its hydrochloride salt |
| Modulators | | (SR-57746A) |
| | C-52 | T-588 |
| | C-53 | 3,4 diaminopyridine |
| | C-54 | CPC-304 |
| | C-55 | CPC-317 |
| | C-56 | PD-176078 |
| | C-57 | cephalosporin ceftriaxone |
| Neuroprotective | C-58 | glatiramer acetate (Copaxone ®) |
| Agents | C-59 | huperzine A |
| | C-60 | 10-methyl-huperzine A |
| | C-61 | 10,10 dimethyl huperzine A |
| | C-62 | huperzine B |
| | C-63 | nicotine |
| | C-64 | epibaticline |
| | C-65 | cytisine |
| | C-66 | lobeline |
| | C-67 | anabasine |
| | C-68 | 7-chloro-5-methyl-1-[2-(4- |
| | | methylpiperazin-1-yl)-2-oxoethyl]-4- |
| | | oxo-3-phenyl-3,5-dihydro-4H- |
| | | pyridazo[4,5-b]indole |
| | C-69 | 4-methyl-1-[7-chloro-3-(3- |
| | | chlorophenyl)-5-methyl-4-oxo-3,5- |
| | | dihydro-4H-pyridazino[4,5-b]indol- |
| | | 1-ylcarbonyl]piperazine |
| Neurotrophic | C-70 | ciliary neurotrophic factor (CNTF) |
| Growth Factors | C-71 | brain derived neurotrophic factor |
| | | (BNDF) |
| | C-72 | recombinant insulin-like growth |
| | | factor (rhIGF-1, Myotrophin ® |
| | | mecasermin, or Somatomedin C) |
| | C-73 | vascular epithelial growth factor |
| | | (VEGF) |
| | C-74 | gilial cell derived neurotrophic factor |
| | | (GDNF) or liatermin |
| | C-75 | neurturin |
| | C-76 | Pigment epithelium derived factor |
| | | (PEDF) |
| | C-77 | FKBO-neuroimmunophilin ligands |
| | C-78 | AIT-082, leteprinim potassium or |
| | | NeotrofinT) |
| | C-79 | buspirone |
| | C-80 | tetracycline |
| | C-81 | minocycline |
| | C-82 | doxycycline |
| | C-83 | emfilermin |
| | C-84 | cardiotrophin-1 (CT-1) |
| | C-85 | NGF |
| | C-86 | neurotrophin-3 (NT-3) |
| | C-87 | neurotrophin-4/5 (NT-4/5) |
| | C-88 | EHT 201 |
| | C-89 | EHT 202 |
| | C-90 | genistein |
| Thyotrophin Releasing | C-91 | RX-77368 |
| Hormone (TRH) | C-92 | MK-771 |
| Modulators | C-93 | JTP-2942 |
| Apoptosis Modulators | C-94 | TCH-346 |
| | C-95 | WHI-P131 |
| N-acetylated, Alpha- | C-96 | GPI-5693 |
| linked Acidic | C-97 | GPI-5000 |
| Dipeptidase Inhibitors | | |
| (NAALADase | | |
| Inhibitors) | | |
| Caspase Inhibitors | C-98 | ZVAD fmk |
| Collagen Synthesis | C-99 | 3-(2-phenyl-2-oxoethyl)-4,5- |
| Modulators | | dimethylthiazolium salt |
| Oxidase Inhibitors | C-100 | nordihydroguaiaretic acid |
| | C-101 | para REV-5901 (L-655238) |
| | C-102 | Bay-X-1005 |
| | C-103 | ML-3000 |
| | C-104 | Zileuton |
| Glutathione Synthase | C-105 | oxothiazolidine carboxylate |
| Stimulants | | (Procysteine ®) |
| Vascular Endothelial | C-106 | ARR 17477 |
| Growth Factor (VEGF) | | |
| Antagonists | | |
| Nitric Oxide Synthase | | |
| Inhibitors | | |
| Protein Kinase | | |
| Inhibitors | | |
| Mast Cell Activation | | |
| Inhibitors | | |
| Interleukin-1 | | |
| converting enzyme | | |
| (ICE) inhibitors | | |
| Antioxidants | C-107 | superoxide dismutase (SOD) |
| | C-108 | recombinant human CuZn-SOD |
| | C-109 | glutathione |
| | C-110 | glutathione peroxidase |
| | C-111 | catalase |
| | C-112 | nitric oxide synthase |
| | C-113 | tocopherol (Vitamin E) |
| | C-114 | ascorbic acid (Vitamin C) |
| | C-115 | selenium |
| | C-116 | acetylcysteine |
| | C-117 | seleginine (Deprenyl ®) |
| | C-118 | pycnogenol |
| | C-119 | co-enzyme Q10 |
| | C-120 | beta carotene |
| | C-121 | PC 01 |
| | C-122 | SC-55858 |
| | C-123 | edaravone |
| | C-124 | iron (III) porphyrins |
| | C-125 | mithramycin |
| | C-126 | chromomycin |
| | C-127 | daunomycin |
| | C-128 | olivomycin |
| | C-129 | WP-631 |
| Immunosuppressants | C-130 | azathioprine |
| | C-131 | cyclophosphamide |
| | C-132 | cyclosporine |
| | C-133 | intravenous immune globulin (IVIG) |
| | C-134 | cytoxan |
| Cytoskeletal Proteins | C-135 | metallothionine |
| Showing Abnormalities | C-136 | apolipoprotein E |
| in ALS | | |
| Adrenocortical Steroids | C-137 | oxandrolone |
| | C-138 | creatine |
| | C-139 | erythropeotin |
| | C-140 | dehydro epiandrosterone (DHEA) |
| Antispastics | C-141 | baclofen (Lioresal ®) |
| | C-142 | tizandidine (Zanaflex ®) |
| | C-143 | benzodiazepines, including |
| | | clonazepam (Klonopin ®) |
| | C-144 | dantrolene (Dantrium ®) |
| | C-145 | dronabinol (Marinol ®) |
| | C-146 | diazepam (Diastat ®) |
| Drugs for Treating | C-147 | AVP-923 (dextromethorphan |
| Emotional Lability | | hydrobromide and quinidine sulfate) |
| (pseudobulbar effect) | | |
| Drugs for Treating | C-148 | morphine |
| Respiratory Difficulties | C-149 | lorazepam |
| Drugs for Treating | C-150 | aspirin |
| General Pain | C-151 | acetaminophen (Tylenol ®) |
| | C-152 | ibuprofen |
| | C-153 | oxycodone (Percocet ®), or with |
| | | aspirin (Percodan ®) |
| | C-154 | tramadol (Ultram ®) |
| Drugs for Treating | C-155 | amitriptylene |
| Depression | C-156 | fluvoxamine (Luvox ®) |
| | C-157 | sertraline (Zoloft ®) |
| | C-158 | fluoxetine (Prozac ®) |
| Drugs for Treating | C-159 | famotidine (Pepcid ®) |
| Gastric Reflux | C-160 | ranitidine (Zantac ®) |
| Drugs for Treating | C-161 | glycopyrrolate (Robinul ®) |
| Excessive Salivation | C-162 | diphenhydramine hydrochloride |
| | | (Benadryl ®) |
| | C-163 | copolamine |
| | C-164 | trihexyphenidyl |
| | C-165 | clonidine |
| | C-166 | propantheline or its bromide salt |
| | C-167 | tropine |
| | C-168 | botulinum toxin type A |
| Drugs for Treating | C-169 | Robitussin ® |
| Thick Phlegm (mucus) | | |
| Drugs for Treating | C-170 | docusate sodium (Colace ®) |
| Constipation | | |
| Drugs for Treating | C-171 | tolterodine (Detrol ®) |
| Urinary Urgency | | |
| Drugs for Treating | C-172 | TA-0910 |
| Spinal Function | | |
| Disorders | | |
| Drugs for Treating | C-173 | ubiquinone |
| Convulsions | C-174 | minocycline |
| Other Drugs | C-175 | indinavir |
| | C-176 | alpha lipoic acid |
| | C-177 | n-acetyl-cysteine (NAC) |
| | C-178 | polyphenols |
| | C-179 | pregnenolone |
| | C-180 | threonine |
| | C-181 | methylcobalamin |

According to another embodiment, combinations of COX 2 inhibitor(s) and second drugs that may be administered, in accordance with the method of the present invention, for the treatment of ALS are represented in the Tables 2A and 2B below:

TABLE 2A

| COX 2 | |
|---|---|
| Inhibitor | Second Drug(s) |
| I | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| II | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| III | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| IV | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| V | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-1 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-2 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C |
| | 177, C-178, C-179, C-180 or C-181. |
| B-3 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-4 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-5 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-6 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-7 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-8 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-9 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-10 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-11 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-12 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-13 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-14 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-15 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-16 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-17 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-18 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-19 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-20 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-21 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-22 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-23 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-24 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-25 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-26 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-27 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-28 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-29 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-30 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-31 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-32 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-33 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-34 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-35 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-36 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-37 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-38 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-39 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-40 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-41 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-42 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-43 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-44 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-45 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-46 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-47 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-48 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-49 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-50 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-51 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-52 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-53 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-54 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-55 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-56 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-57 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-58 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-59 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-60 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-61 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-62 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-63 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-64 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-65 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-66 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-67 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-68 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-69 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-70 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-71 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-72 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-73 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-74 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-75 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-76 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-77 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-78 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-79 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-80 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-81 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-82 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-83 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-84 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-85 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-86 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-87 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-88 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-89 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-90 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-91 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-92 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-93 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-94 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-95 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-96 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-97 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-98 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-99 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-100 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-101 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-102 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-103 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-104 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-105 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-106 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-107 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-108 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-109 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-110 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-111 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-112 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-113 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-114 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-115 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-116 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-117 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-118 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-119 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-120 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-121 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-122 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-123 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-124 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-125 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-126 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-127 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-128 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-129 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-130 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-131 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-132 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-133 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-134 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-135 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-136 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-137 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-138 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-139 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-140 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-141 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-142 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-143 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-144 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-145 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-146 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-147 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-148 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-149 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-150 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-151 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-152 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-153 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-154 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-155 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C, 115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-156 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-157 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-158 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-159 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-160 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-161 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-162 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-163 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-164 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-165 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-166 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-167 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-168 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-169 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-170 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-171 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-172 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-173 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-174 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-175 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-176 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-177 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-178 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-179 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-180 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-181 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-182 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-183 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-184 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-185 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-186 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-187 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-188 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-189 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-190 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-191 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-192 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-193 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-194 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-195 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-196 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-197 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-198 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-199 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-200 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-201 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-202 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-203 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-204 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-205 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-206 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-207 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-208 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-209 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-210 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-211 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-212 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-213 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-214 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-215 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-216 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-217 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-218 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-219 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-220 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-221 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-222 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-223 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-224 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-225 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-226 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-227 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-228 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-229 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-230 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-231 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-232 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |
| B-233 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81, C-82, C-83, C-84, C-85, |
| | C-86, C-87, C-88, C-89, C-90, C-91, C-92, C-93, C-94, C-95, |
| | C-96, C-97, C-98, C-99, C-C-101, C-102, C-103, C-104, C- |
| | 105, C-106, C-107, C-108, C-109, C-110, C-111, C-112, C- |
| | 113, C-114, C-115, C-116, C-117, C-118, C-119, C-120, C- |
| | 121, C-122, C-123, C-124, C-125, C-126, C-127, C-128, C- |
| | 129, C-130, C-131, C-132, C-133, C-134, C-135, C-136, C- |
| | 137, C-138, C-139, C-140, C-141, C-142, C-143, C-144, C- |
| | 145, C-146, C-147, C-148, C-149, C-150, C-151, C-152, C- |
| | 153, C-154, C-155, C-156, C-157, C-158, C-159, C-160, C- |
| | 161, C-162, C-163, C-164, C-165, C-166, C-167, C-168, C- |
| | 169, C-170, C-171, C-172, C-173, C-174, C-175, C-176, C- |
| | 177, C-178, C-179, C-180 or C-181. |

TABLE 2B

| Second Drug | COX 2 Inhibitor |
|---|---|
| Glutamate | In combination with one or more of I, II, III, IV, V, B- |
| Antagonists | 1, B-2, B-3, B-4, B-5, B-6, B-7, B-8, B-9, B-10, B-11, |
| | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| NMDA | In combination with one or more of I, II, III, IV, V, B- |
| Receptor | 1, B-2, B-3, B-4, B-5, B-6, B-7, B-8, B-9, B-10, B-11, |
| Antagonists | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| AMPA | In combination with one or more of I, II, III, IV, V, B- |
| Receptor | 1, B-2, B-3, B-4, B-5, B-6, B-7, B-8, B-9, B-10, B-11, |
| Antagonists | B-12, B-13, B-14, B-15, B-16, B-17, B-18, B-19, B-20, |
| and | B-21, B-22, B-23, B-24, B-25, B-26, B-27, B-28, B-29, |
| Desensitizers | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Metabotropic | In combination with one or more of I, II, III, IV, V, B- |
| Receptor | 1, B-2, B-3, B-4, B-5, B-6, B-7, B-8, B-9, B-10, B-11, |
| Agonists | B-12, B-13, B-14, B-15, B-16, B-17, B-18, B-19, B-20, |
| and | B-21, B-22, B-23, B-24, B-25, B-26, B-27, B-28, B-29, |
| Antagonists | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Branched | In combination with one or more of I, II, III, IV, V, B- |
| Chain | 1, B-2, B-3, B-4, B-5, B-6, B-7, B-8, B-9, B-10, B-11, |
| Amino Acids | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Neurotrophic | In combination with one or more of I, II, III, IV, V, B- |
| Growth Factors | 1, B-2, B-3, B-4, B-5, B-6, B-7, B-8, B-9, B-10, B-11, |
| | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Antioxidants | In combination with one or more of I, II, III, IV, V, B- |
| | 1, B-2, B-3, B-4, B-5, B-6, B-7, B-8, B-9, B-10, B-11, |
| | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Immunosup- | In combination with one or more of I, II, III, IV, V, B-1, |
| pressants | B-2, B-3, B-4, B-5, B-6, B-7, B-8, B-9, B-10, B-11, |
| | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Cytoskeletal | In combination with one or more of I, II, III, IV, V, B- |
| Proteins | 1, B-2, B-3, B-4, B-5, B-6, B-7, B-8, B-9, B-10, B-11, |
| Showing | B-12, B-13, B-14, B-15, B-16, B-17, B-18, B-19, B-20, |
| Abnormalities | B-21, B-22, B-23, B-24, B-25, B-26, B-27, B-28, B-29, |
| in ALS | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Adrenocortical | In combination with one or more of I, II, III, IV, V, B- |
| Steroids | 1, B-2, B-3, B-4, B-5, B-6, B-7, B-8, B-9, B-10, B-11, |
| | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-B33, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Antispastics | In combination with one or more of I, II, III, IV, V, B- |
| | 1, B-2, B-3, B-4, B-5, B-6, B-7, B-8, B-9, B-10, B-11, |
| | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| Emotional | B-12, B-13, B-14, B-15, B-16, B-17, B-18, B-19, B-20, |
| Lability | B-21, B-22, B-23, B-24, B-25, B-26, B-27, B-28, B-29, |
| (pseudobulbar | B-30, B-31, B-32, B-33, B-34, B-35, B-36, B-37, B-38, |
| effect) | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| Respiratory | B-12, B-13, B-14, B-15, B-16, B-17, B-18, B-19, B-20, |
| Difficulties | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| General Pain | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| Depression | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| Gastric Reflux | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| Excessive | B-12, B-13, B-14, B-15, B-16, B-17, B-18, B-19, B-20, |
| Salivation | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| Thick | B-12, B-13, B-14, B-15, B-16, B-17, B-18, B-19, B-20, |
| Phlegm | B-21, B-22, B-23, B-24, B-25, B-26, B-27, B-28, B-29, |
| (mucus) | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| Constipation | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Drugs for | In combination with one or more of I, II, III, IV, V, B- |
| Treating | 1, B-2, B-3, B-4, B-5, B-6, B-7, B-8, B-9, B-10, B-11, |
| Urinary | B-12, B-13, B-14, B-15, B-16, B-17, B-18, B-19, B-20, |
| Urgency | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |
| Other Drugs | In combination with one or more of I, II, III, IV, V, B- |
| | 1, B-2, B-3, B-4, B-5, B-6, B-7, B-8, B-9, B-10, B-11, |
| | B-12, B-13, B-14, B-15, B-16, B-17, B-18, B-19, B-20, |
| | B-21, B-22, B-23, B-24, B-25, B-26, B-27, B-28, B-29, |
| | B-30, B-31, B-32, B-33, B-34, B-35, B-36, B-37, B-38, |
| | B-39, B-40, B-41, B-42, B-43, B-44, B-45, B-46, B-47, |
| | B-48, B-49, B-50, B-51, B-52, B-53, B-54, B-55, B-56, |
| | B-57, B-58, B-59, B-60, B-61, B-62, B-63, B-64, B-65, |
| | B-66, B-67, B-68, B-69, B-70, B-71, B-72, B-73, B-74, |
| | B-75, B-76, B-77, B-78, B-79, B-80, B-81, B-82, B-83, |
| | B-84, B-85, B-86, B-87, B-88, B-89, B-90, B-91, B-92, |
| | B-93, B-94, B-95, B-96, B-97, B-98, B-99, B-100, B- |
| | 101, B-102, B-103, B-104, B-105, B-106, B-107, B- |
| | 108, B-109, B-110, B-111, B-112, B-113, B-114, B- |
| | 115, B-116, B-117, B-118, B-119, B-120, B-121, B- |
| | 122, B-123, B-124, B-125, B-126, B-127, B-128, B- |
| | 129, B-130, B-131, B-132, B-133, B-134, B-135, B- |
| | 136, B-137, B-138, B-139, B-140, B-141, B-142, |
| | B-143, B-144, B-145, B-146, B-147, B-148, B-149, B- |
| | 150, B-151, B-152, B-153, B-154, B-155, B-156, B- |
| | 157, B-158, B-159, B-160, B-161, B-162, B-163, B- |
| | 164, B-165, B-166, B-167, B-168, B-169, B-170, |
| | B-171, B-172, B-173, B-174, B-175, B-176, B-177, B- |
| | 178, B-179, B-180, B-181, B-182, B-183, B-184, B- |
| | 185, B-186, B-187, B-188, B-189, B-190, B-191, B- |
| | 192, B-193, B-194, B-195, B-196, B-197, B-198, B- |
| | 199, B-200, B-201, B-202, B-203, B-204, B-205, B- |
| | 206, B-207, B-208, B-209, B-210, B-211, B-212, B- |
| | 213, B-214, B-215, B-216, B-217, B-218, B-219, B- |
| | 220, B-221, B-222, B-223, B-224, B-225, B-226, B- |
| | 227, B-228, B-229, B-230, B-231, B-232 or B-233. |

According to another embodiment, the invention is directed to a novel method for the treatment of ALS comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, wherein said COX 2 inhibitor comprises a chromene that is a substituted benzopyran, or is a chroman.

According to yet another embodiment, the invention is directed to a novel method for the treatment of ALS comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is selected from the group consisting of substituted benzothiopyrans, dihydroquinolines, or dihydronaphthalenes having the general Formula (I):

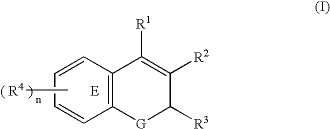

or an isomer, a pharmaceutically acceptable salt, an ester, or a prodrug thereof,

wherein n is an integer which is 0, 1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with carbon atoms to which it is attached and the remainder of the ring E forms a naphthyl radical.

According to another embodiment, the invention is also directed to a novel method for the treatment of ALS comprising administering, to a subject in need thereof, a cyclooxygenase-2 (COX 2) inhibitor in a first amount and a second drug in a second amount, wherein said first amount together with said second amount is a therapeutically effective amount of said COX 2. inhibitor and said second drug, and wherein said COX 2 inhibitor has the general Formula (II):

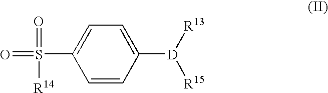

or an isomer, a pharmaceutically acceptable salt, ester, or prodrug thereof, provided that Formula (II) is not celecoxib (B-18) or rofecoxib (B-21) as listed in Table IA above,

wherein:

D is selected from the group consisting of partially unsaturated or saturated heterocyclyl and partially unsaturated or saturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is methyl or amino; and

R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

According to another embodiment, the invention is also directed to a novel method for the treatment of ALS comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor has the general Formula (II):

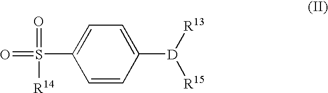

or an isomer, a pharmaceutically acceptable salt, ester, or prodrug thereof,

wherein:

D is selected from the group consisting of partially unsaturated or saturated heterocyclyl and partially unsaturated or saturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is methyl or amino; and

R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl; and,

wherein said second drug comprises C-1, C-2, C-3, C-4, C-5, C-6, C-7, C-8, C-9, C-10, C-11, C-12, C-13, C-14, C-15, C-16, C-17, C-18, C-19, C20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C—C-101, C-102, C-103, C-104, C-105, C-106, C-107, C-108, C-109, C-110, C-111, C-112, C-113, C-114, C-115, C-116, C-117, C-118, C-119, C-120, C-121, C-122, C-123, C-124, C-125, C-126, C-127, C-128, C-129, C-130, C-131, C-132, C-133, C-134, C-135, C-136, C-137, C-138, C-139, C-140, C-141, C-142, C-143, C-144, C-145, C-146, C-147, C-148, C-149, C-150, C-151, C-152, C-153, C-154, C-155, C-156, C-157, C-158, C-159, C-160, C-161, C-162, C-163, C-164, C-165, C-166, C-167, C-168, C-169, C-170, C-171, C-172, C-173, C-174, C-175, C-176, C-177, C-178, C-179, C-180, C-181, or an isomer, a pharmaceutically acceptable salt, ester, or prodrug thereof.

According to another embodiment, the invention is also directed to a novel method for the treatment of ALS comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor has the general Formula (II):

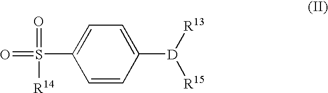

or an isomer, a pharmaceutically acceptable salt, ester, or prodrug thereof,

wherein:

D is selected from the group consisting of partially unsaturated or saturated heterocyclyl and partially unsaturated or saturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is methyl or amino; and

R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl; and,

wherein said second drug is a glutamate antagonist, a neurotrophic growth factor, an antioxidant, an immunosuppressant, a cytoskeletal protein, an adrenocortical steroid, or an antispastic.

According to another embodiment, the present invention is also directed to a novel method for the treatment of ALS comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor comprises a phenylacetic acid derivative represented by the general Formula (III):

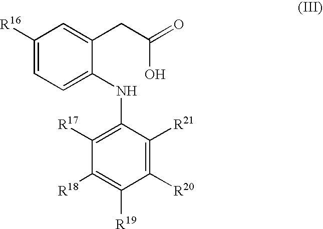

or an isomer, a pharmaceutically acceptable salt, ester, or prodrug thereof,

wherein:

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl,

provided that R¹⁷, R¹⁸, R¹⁹ and R²⁰ are not all fluoro when R¹⁶ is ethyl and R¹⁹ is H.

According to another embodiment, the invention is directed to a method for the treatment of Amyotrophic Lateral Sclerosis comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (IV):

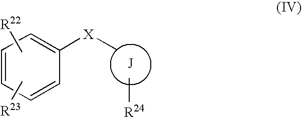

or an isomer, a pharmaceutically acceptable salt, ester, or prodrug thereof, wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the invention is directed to a method for the treatment of Amyotrophic Lateral Sclerosis comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor has the structural Formula (V):

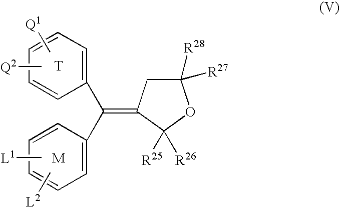

or an isomer, a pharmaceutically acceptable salt, ester, or prodrug thereof, wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

The present invention is also directed to a novel method of treating, improving or preventing a cyclooxygenase-2 mediated disorder in a subject, said method comprising treating the subject having or susceptible to said disorder with a therapeutically-effective amount of a pharmaceutical composition comprising any one of the cyclooxygenase-2-selective inhibitors described above and any one of the second drugs or categories of second drugs described above.

### DETAILED DESCRIPTION OF THE PREFERRED EMBODIMENTS

In accordance with the present-invention, it has been discovered that ALS can be treated by administering one or more cyclooxygenase-2 selective inhibitor(s) disclosed in Tables 1 and 1A above to subject(s) in need of such treatment, in combination with one or more second drug(s) disclosed in Table 2 or within the general categories of drugs disclosed in Table 2. The amounts of cyclooxygenase-2-selective inhibitor(s) and second drug(s) used in the treatment of ALS are selected so that the combined amount is a therapeutically effective amount for the treatment, inhibition and/or prevention of ALS, preferably with minimal side effects.

The following definitions are provided in order to aid the reader in understanding the detailed description of the present invention.

As used herein, the term “purified” means partially purified and/or completely purified. Thus a “purified composition” may be either partially purified or completely purified. The COX 2 inhibitor(s), as well as the second drug(s), useful in the inventive method for treating ALS, can be of any purity and quality such that the combinatiosn fo the COX 2 inhibitor(s) and second drug(s) is pharmaceutically acceptable.

As used herein, the term “cyclooxygenase-2 inhibitor”, embraces compounds which selectively inhibit cyclooxygenase-2 over cyclooxygenase-1, and also includes pharmaceutically acceptable salts of those compounds.

In practice, the selectivity of a COX 2 inhibitor varies depending upon the condition under which the test is performed and on the inhibitors being tested. However, for the purposes of this specification, the selectivity of a COX 2 inhibitor can be measured as a ratio of the in vitro or in vivo IC₅₀ value for inhibition of COX 1, divided by the IC₅₀ value for inhibition of COX 2 (COX 1 IC₅₀/COX 2 IC₅₀). A COX 2 selective inhibitor is any inhibitor for which the ratio of COX 1 IC₅₀ to COX 2 IC₅₀ is greater than 1, preferably greater than 1.5, more preferably greater than 2, even more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity.

Preferred cyclooxygenase-2 selective inhibitors of the present invention have a cyclooxygenase-2 IC₅₀ of less than about 5 μM, more preferably less than about 1 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors, as well as compounds that act as prodrugs for the second drug. As used herein in reference to COX 2 selective inhibitors and second drugs, the term “prodrug” refers to a chemical compound that is converted into an active COX 2 selective inhibitor by metabolic processes within the body. One example of a prodrug for a COX 2 selective inhibitor is parecoxib, which is a therapeutically effective prodrug of the tricyclic cyclooxygenase-2 selective inhibitor valdecoxib. An example of a preferred COX 2 selective inhibitor prodrug is sodium parecoxib.

As used herein, an “effective amount” or “therapeutically effective amount” means the dose or effective amount to be administered to a patient and the frequency of administration to the subject which is sufficient to obtain a therapeutic effect as readily determined by one of ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically effective” indicates the capability of a combination of agents to prevent, or reduce the severity of, the disorder or its undesirable symptoms, while avoiding adverse side effects typically associated with alternative therapies. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The amounts of COX 2 selective inhibitor(s) and second drug(s) that are used in the subject method may be amounts that, together, are sufficient to constitute an effective amount for ALS treatment or prevention. In the present method, the amount of COX 2 selective inhibitor that is used in the novel method of treatment preferably ranges from about 0.001 to about 100 milligrams per day per kilogram of body weight of the subject (mg/day·kg), more preferably from about 0.05 to about 50 mg/day·kg, even more preferably from about 1 to about 20 mg/day·kg. The amount of the second drug, used in combination with the COX 2 selective inhibitor, preferably ranges from about 0.001 to about 100 milligrams per day per kilogram of body weight of the subject (mg/day·kg), more preferably from about 0.1 to about 10 mg/day·kg, even more preferably from about 0.5 to about 2 mg/day·kg. Preferably, the weight ratio of the amount(s) of COX 2 inhibitor(s) administered to the amount(s) of second drug(s) administered is from about 0.002 to about 10, more preferably from about 0.1 to about 5.

When the COX 2 selective inhibitor comprises rofecoxib, it is preferred that the amount used is within a range of from about 0.15 to about 1.0 mg/day·kg, and even more preferably from about 0.18 to about 0.4 mg/day·kg. When the COX 2 selective inhibitor comprises etoricoxib, it is preferred that the amount used is within a range of from about 0.5 to about 5 mg/day·kg, and even more preferably from about 0.8 to about 4 mg/day·kg. When the COX 2 selective inhibitor comprises celecoxib, it is preferred that the amount used is within a range of from about 1 to about 20 mg/day·kg, even more preferably from about 1.4 to about 8.6 mg/day·kg, and yet more preferably from about 2 to about 3 mg/day·kg. When the COX 2 selective inhibitor comprises valdecoxib, it is preferred that the amount used is within a range of from about 0.1 to about 5 mg/day·kg, and even more preferably from about 0.8 to about 4 mg/day·kg. When the COX 2 selective inhibitor comprises parecoxib, it is preferred that the amount used is within a range of from about 0.1 to about 5 mg/day·kg, and even more preferably from about 1 to about 3 mg/day·kg.

In terms of absolute daily dosages, when the COX 2 selective inhibitor comprises rofecoxib, it is preferred that the amount used is from about 10 to about 75 mg/day, more preferably from about 12.5 to about 50 mg/day. When the COX 2 selective inhibitor comprises etoricoxib, it is preferred that the amount used is from about 50 to about 100 mg/day, more preferably from about 60 to about 90 mg/day. When the COX 2 selective inhibitor comprises celecoxib, it is preferred that the amount used is from about 100 to about 1000 mg/day, more preferably from about 200 to about 800 mg/day. When the COX 2 selective inhibitor comprises valdecoxib, it is preferred that the amount used is from about 5 to about 100 mg/day, more preferably from about 10 to about 60 mg/day. When the COX 2 selective inhibitor comprises parecoxib, it is preferred that the amount used is within a range of from about 10 to about 100 mg/day, more preferably from about 20 to about 80 mg/day.

The COX 2 selective inhibitor(s) and second drugs that are described above can be provided in a therapeutic composition so that the preferred amounts thereof is/are supplied by a single dosage, a single capsule for example, or, by up to four, or more, single dosage forms.

In one embodiment of the invention, the COX 2 inhibitor(s) and the second drug(s) may be administered substantially simultaneously, meaning that both agents may be provided in a single dosage, for example by mixing the agents and incorporating the mixture into a single capsule. Otherwise, the COX 2 inhibitor(s) and second drug(s) may be administered substantially simultaneously by administration in separate dosages within a short time period, for example within 5 minutes or less. Alternatively, the COX 2 inhibitor(s) and second drug(s) may be administered sequentially, meaning that separate dosages, and possibly even separate dosage forms of the COX 2 inhibitor(s) and second drugs(s) may be administered at separate times, for example on a staggered schedule but with equal frequency of administration of the COX 2 inhibitor(s) and the second drug(s). Of course, it is also possible that the COX 2 inhibitor(s) may be administered either more or less frequently than the second drug(s). In any case, it is preferable that, among successive time periods of a sufficient length, for example one day, the weight ratio of the COX 2 inhibitor(s) administered to the weight ratio of the second drug(s) administered remains constant.

In some cases, it may be desireable to administer a combination of second drugs listed in Table 2 and/or described herein, or a single second drug listed in Table 2 and/or described herein in combination with one or more drugs not listed in Table 2 or described herein. For example, second drugs that may be advantageously combined include riluzole and alpha tocopherol, which may slow ALS progression compared to the administration of riluzole alone. Also, the drug known as AVP-923 provides an example of a second drug (i.e., the NMDA receptor dextromethorphan) that may be combined with an enzyme that inhibits the dextromethorphan with which such enzyme is co-administered. A further example of a second drug combination that may be beneficial is the administration of huperzine and nicotinic compounds, as described, for example, in WO-00211712.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in connection with use of the method(s) of the present invention are the isomeric forms and tautomers and the pharmaceutically-acceptable salts of the cyclooxygenase-2 selective inhibitors and the second drugs. Isomers of COX 2 inhibitors and second drugs include their diastereomers, enantiomers, and racemates as well as their structural isomers. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric, and galacturonic acids.

Suitable pharmaceutically-acceptable base addition salts of compounds used in connection with the method(s) of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethyl amine, diethyl amine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention. Pharmaceutically acceptable esters include, but are not limited to, the alkyl esters of both the COX 2 inhibitors and the second drugs. For example, the second drug levodopa may be administered as its methyl ester or its ethyl ester.

The method of the present invention is useful for, but not limited to, the prevention, inhibition, and/or treatment of ALS.

As used herein, the terms “ALS” and “cyclooxygenase-2 mediated disorder” are meant to include, without limitation, each of the symptoms associated with Amyotrophic Lateral Sclerosis that is mentioned in this application.

The present method includes the treatment, inhibition and/or prevention of a cyclooxygenase-2 mediated disorder in a subject, where the method comprises treating the; subject having or susceptible to the disorder with a combined therapeutically-effective amount of the cyclooxygenase-2 selective inhibitor(s) and second drug(s) that are described in this specification. This method is useful where the cyclooxygenase-2 mediated disorder is ALS.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of undesirable symptoms associated with ALS. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has pain, inflammation and/or any one of the known inflammation-associated disorders. The subject is typically a human subject.

For methods of prevention, the subject is any human or animal subject, and preferably is a subject that is in need of prevention and/or treatment of ALS. The subject may be a human subject who is at risk for ALS. The subject may be at risk for ALS due to genetic predisposition, lifestyle, diet, exposure to disorder-causing agents, exposure to pathogenic agents and the like.

In connection with the inventive method, the COX 2 pharmaceutical composition(s) and second drug(s) may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrase “administration” in defining the use of both a cyclooxygenase-2 inhibitor agent and a second drug is intended to embrace administration of each agent in a manner and in a regimen that will provide beneficial effects of the drug combination therapy, and is intended as well to embrace co-administration of 2 or more of the COX 2 agents in a substantially simultaneous manner and/or 2 or more of the second drugs in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from the constituent COX 2 agent and second drug used in combination.

The phrases “therapeutically-effective” and “effective for the treatment, prevention, or inhibition”, are intended to qualify the amount of each COX 2 agent and each second drug for use in the COX 2 therapy which will achieve the goal of reduction of the severity and/or frequency of incidence of ALS associated symptoms, while avoiding adverse side effects typically associated with alternative therapies.

The terms “hydrido” and “H” denote a single hydrogen atom. This hydrido radical may be attached, for example, to an oxygen atom to form a hydroxyl radical or two hydrido radicals may be attached to a carbon atom to form a methylene (—CH₂—) radical. Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms.

Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations.

The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl and cyclohexenyl.

The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The terms “heterocyclo”, “heterocyclyl”, and “heterocycle” embrace saturated, partially unsaturated and unsaturated heteroatom-containing ring-shaped radicals, where the heteroatoms may be selected from nitrogen, sulfur and oxygen. Examples of saturated heterocyclo radicals include saturated 3 to 6-membered heteromonocyclic groups containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclo radicals include dihydrothiophene, dihydropyran, dihydrofuran and dihydrothiazole.

The term “heteroaryl” embraces unsaturated heterocyclo radicals. Examples of unsaturated heterocyclo radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclo group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclo group containing 1 to 2 oxygen atoms and 1 to 3 nitrogen atoms (e.g. benzoxazolyl, benzoxadiazolyl, etc.); unsaturated 3 to 6-membered heteromonocyclic: group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclo group containing 1 to 2 sulfur atoms and 1 to 3 nitrogen atoms (e.g., benzothiazolyl, benzothiadiazolyl, etc.) and the like. The term also embraces radicals where heterocyclo radicals are fused with aryl radicals. Examples of such fused bicyclic radicals include benzofuran, benzothiophene, benzopyran, and the like. Said “heterocyclo group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing, an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl and trifluoroacetyl.

The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, haloalkyl and haloalkoxy. The terms benzyl and phenylmethyl are interchangeable.

The term “heterocycloalkyl” embraces saturated and partially unsaturated heterocyclo-substituted alkyl radicals, such as pyrrolidinylmethyl, and heteroarylsubstituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “aralkoxy” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups that have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkylaminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂. The term “alkylaminocarbonyl” denotes an aminocarbonyl group that has been substituted with one or two alkyl radicals on the amino nitrogen atom. Preferred are “N-alkylaminocarbonyl” and “N,N-dialkylaminocarbonyl” radicals. More preferred are “lower N-alkylaminocarbonyl” and “lower N,N-dialkylaminocarbonyl” radicals with lower alkyl portions as defined above. The term “aminocarbonylalkyl” denotes a carbonylalkyl group that has been substituted with an amino radical on the carbonyl carbon atom.

The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

As used herein, the term “carbocycle” means a hydrocarbon ring radical. Carbocyclic rings are monocyclic or are fused, bridged, or spiro polycyclic rings. Unless otherwise specified, monocyclic rings contain from 3 to about 9 atoms, preferably from about 4 to about 7 atoms, and most preferably 5 or 6 atoms. Polycyclic rings contain from about 7 to about 17 atoms, preferably from about 7 to about 14 atoms, and most preferably 9 or 10 atoms. Carbocyclic rings (carbocycles) may be substituted or unsubstituted.

The cyclooxygenase-2 selective inhibitor of the present invention can be, for example, the COX 2 selective inhibitor [2-(2,4-dichloro-6-ethyl-3,5-dimethyl-phenylamino)-5-propyl-phenyl]-acetic acid, having Formula B-1, or an isomer, pharmaceutically acceptable salt, ester, or prodrug thereof.

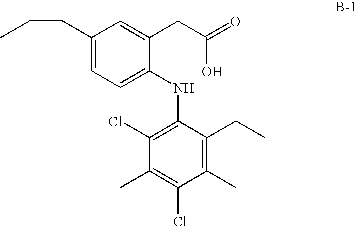

In another embodiment of the invention the cyclooxygenase-2 selective inhibitor can be the COX 2 selective inhibitor RS 57067 or 6-[[5-(4-chlorobenzoyl)-1,4-dimethyl-1H-pyrrol-2-yl]methyl]-3(2H)-pyridazinone, having Formula B-2 (CAS registry number 179382-91-3), or an isomer, a pharmaceutically acceptable salt, ester, or prodrug thereof.

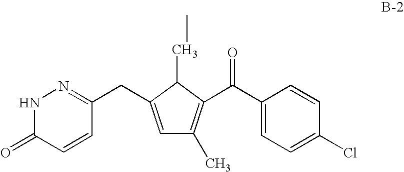

In a preferred embodiment of the invention the cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having a structure shown by general Formulas (I)-(V), shown below, and possessing, by way of example and not limitation, the structures disclosed in Table 1, including the diastereomers, enantiomers, racemates, tautomers, salts, esters, amides and prodrugs thereof.

Furthermore, benzopyran COX 2 selective inhibitors useful in the practice of the present invention are described in U.S. Pat. Nos. 6,034,256 and 6,077,850.

Formula (I) is:

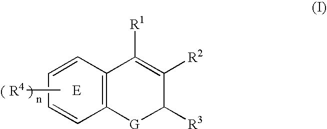

or an isomer, a pharmaceutically acceptable salt, ester, or prodrug thereof;

wherein n is an integer which is 0, 1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof;

wherein:

n is an integer which is 0, 1, 2, 3 or 4;

wherein:

G is O, S or NR^{b};

R¹ is H;

R^{b} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino; heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

The cyclooxygenase-2 selective inhibitor may also be a compound of Formula (I), or an isomer, a pharmaceutically acceptable salt, ester, or prodrug thereof; wherein:

n is an integer which is 0, 1, 2, 3 or 4;

G is oxygen or sulfur;

R¹ is H;

R² is carboxyl, lower alkyl lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof, wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

The cyclooxygenase-2 selective inhibitor used in connection with the method(s) of the present invention can also be a compound having the structure of Formula (I) or an isomer, a pharmaceutically acceptable salt, ester, or prodrug thereof, wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R¹¹ is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia):

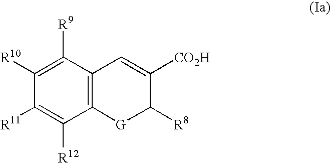

or an isomer, a pharmaceutically acceptable salt, ester, or prodrug thereof.

The cyclooxygenase-2 selective inhibitor used in connection with the method(s) of the present invention can also be a compound of having the structure of Formula (Ia) or an isomer, a pharmaceutically acceptable salt, ester, or prodrug thereof; wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

The present invention is also directed to a novel method for the treatment of ALS comprising administering to a subject in need thereof a therapeutically effective amount of a cyclooxygenase-2 selective inhibitor comprising BMS-347070 (B-74), ABT 963 (B-25), NS-398 (B-26), L-745337 (B-214), RWJ-63556 (B-215), or L-784512 (B-216).

Of the COX 2 inhibitors listed in Table IA, those listed in Table 3 are chromene COX 2 inhibitors as indicated below:

TABLE 3
Examples of Chromene COX 2 Selective Inhibitors

| No. | Structure (chromene COX 2 Inhibitor) |
|---|---|
| B-3 | 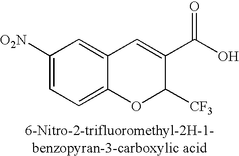 |
| B-4 | 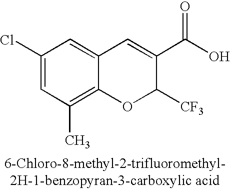 |
| B-5 | 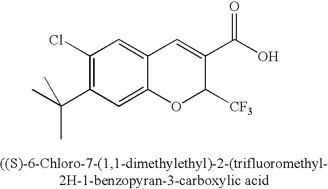 |
| B-6 | 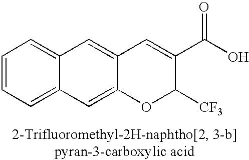 |
| B-7 | 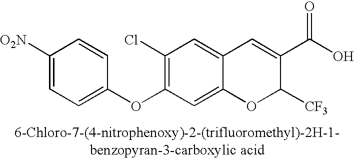 |
| B-8 | 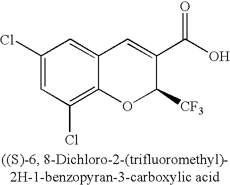 |
| B-9 | 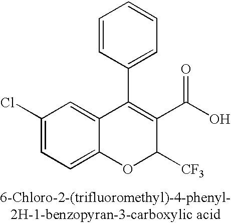 |
| B-10 | 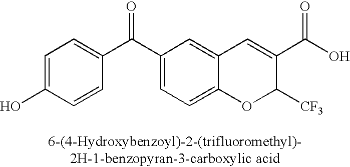 |
| B-11 | 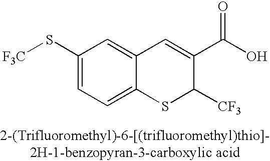 |
| B-12 | 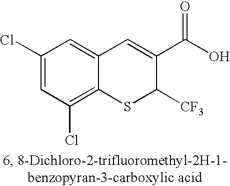 |
| B-13 | 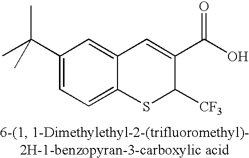 |
| B-14 | 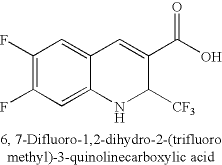 |
| B-15 | 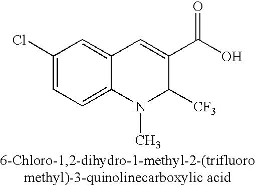 |
| B-16 | 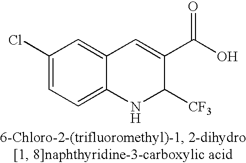 |
| B-17 | 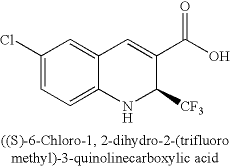 |

In a further preferred embodiment of the invention, the cyclooxygenase inhibitor, when used in combination with any of the second drugs C-1 to C-181 or categories of second drugs (e.g., glutamate antagonists) listed in Table 2, can be selected from the class of tricyclic cyclooxygenase-2 selective inhibitors represented by the general structure of Formula (II):

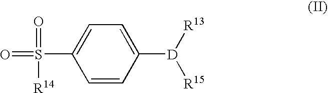

or an isomer, a pharmaceutically acceptable salt, ester, or prodrug thereof, wherein:

D is selected from the group consisting of partially unsaturated or unsaturated heterocyclyl and partially unsaturated or unsaturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is selected from the group consisting of methyl or amino; and

R¹⁵ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In a still more preferred embodiment of the invention, the tricyclic cyclooxygenase-2 selective inhibitor(s), for use in connection with the method(s) of the present invention and in combination with any of the second drugs C-1 to C-181 or categories of second drugs (e.g., glutamate antagonists) listed in Table 2, are represented by the above Formula (II) and are selected from the group of compounds consisting of celecoxib (B-18), valdecoxib (B-19), deracoxib (B-20), rofecoxib (B-21), etoricoxib (MK-663; B-22), JTE-522 (B-23), or an isomer, a pharmaceutically acceptable salt, ester, or prodrug thereof. Examples of tricyclic COX 2 selective inhibitors are provided in Table 4 below:

TABLE 4
Examples of Tricyclic COX 2 Selective Inhibitors

| No. | Structure (Tricyclic COX 2 Inhibitors) |
|---|---|
| B-18 | 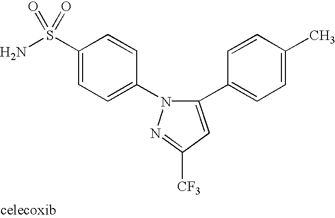 |
| B-19 | 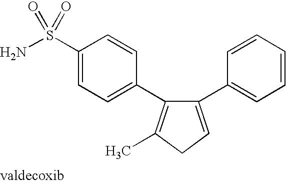 |
| B-20 | 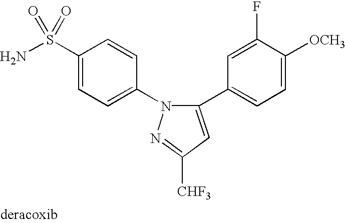 |
| B-21 | 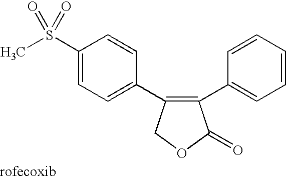 |
| B-22 | 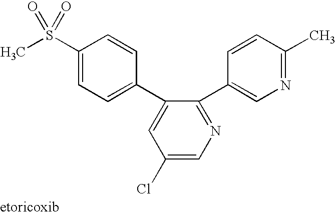 |
| B-23 | 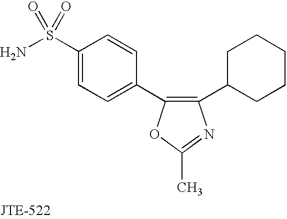 |

In an even more preferred embodiment of the invention, the COX 2 selective inhibitor, when used in combination with any of the second drugs C-1 to C-181 or categories of second drugs (e.g., glutamate antagonists) listed in Table 2, is selected from the group consisting of celecoxib, rofecoxib and etoricoxib.

In another preferred embodiment of the invention, parecoxib, (B-24), which is a therapeutically effective prodrug of the tricyclic cyclooxygenase-2 selective inhibitor valdecoxib, (B-19), may be advantageously employed as a source of a cyclooxygenase inhibitor (See, e.g., U.S. Pat. No. 5,932,598) in connection with the method(s) in the present invention.

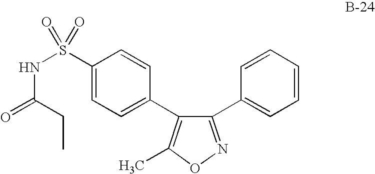

A preferred form of parecoxib is sodium parecoxib.

In another preferred embodiment of the invention, the compound ABT-963 having the formula (B-25) that has been previously described in International Publication number WO-0024719, is another tricyclic cyclooxygenase-2 selective inhibitor which may be advantageously employed in connection with the method(s) of the present invention.

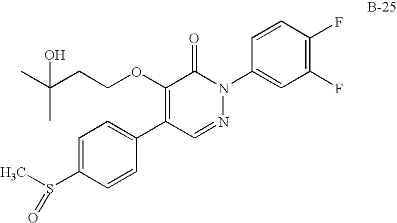

Another preferred cyclooxygenase-2 selective inhibitor that is useful in connection with the method(s) of the present invention is N-(2-cyclohexyloxynitrophenyl)-methane sulfonamide (NS-398)—having a structure shown below as B-26. Applications of this compound have been described by, for example, Yoshimi, N. et al., in Japanese J. Cancer Res., 90(4):406-412 (1999); Falgueyret, J. -P. et al., in Science Spectra, available at: http://www.gbhap.com/Science_Spectra/20-1-article.htm (06/06/2001); and Iwata, K. et al., in Jpn. J. Pharmacol., 75(2):191-194 (1997).

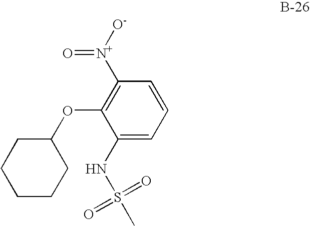

Other compounds that are useful for the cyclooxygenase-2 selective inhibitor in connection with the method(s) of the present invention include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)-4-(4-fluorophenyl)-5-(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-1H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)-4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl) phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2,4-dichloro-6-methyl-phenylamino)-5-ethyl-phenyl]-acetic acid or COX 189 (B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3-(3,4-difluoro-phenoxy)-4-(4-methanesulfonyl-phenyl)-5-methyl-5-(2,2,2-trifluoro-ethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or T-614 (B-224);

D-1367 (B-225);

L-748731 (B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

2-(6-dioxo-9H-purin-8-yl)cinnamic acid (B-231);

S-2474 (B-232); or

Meloxicam (B-233)

or an isomer, a pharmaceutically acceptable salt, ester or prodrug thereof, respectively.

Certain subgroups of the above-noted COX 2 inhibitors may be preferred for the treatment of ALS which include, but are not limited to, B-1 to B-5, B-6 to B-10, B-11 to B-15, B-16 to B-20, B-21 to B-25, B-26 to B-30, B-31 to B-35, B-36-B-40, B-41 to B-45, B-46 to B-50, B-51 to B-55, B-56 to B-60, B-61 to B-65, B-66 to B-70, B-71 to B-75, B-76 to B-80, B-81 to B-85, B-—B-86 to B-90, B-91 to B-95, B-96 to B-100, B-101 to B-105, B-106 to B-110, B-111 to B-115, B-116 to B-120, B-121 to B-125, B-126 to B-130, B-131 to B-135, B-136 to B-140, B-141 to B-145, B-146 to B-150, B-151 to B-155, B-156 to B-160, B-161 to B-165, B-166 to B-170, B-171 to B-175, B-176 to B-180, B-181 to B-185, B-186 to B-190, B-191 to B-195, B-196 to B-200, B-201 to B-205, B-206 to B-210, B-211 to B-215, B-216 to B-220, B-221 to B-225, B-226 to B-230, B-231 to B-233 or combinations thereof.

In a further preferred embodiment of the invention, the cyclooxygenase inhibitor used in connection with the method(s) of the present invention can be selected from the class of phenylacetic acid derivative cyclooxygenase-2 selective inhibitors represented by the general structure of Formula (III):

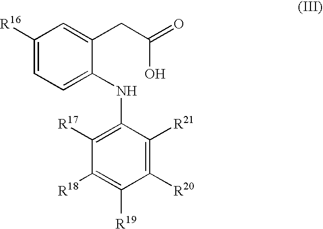

or an isomer, a pharmaceutically acceptable salt, ester, or prodrug thereof;

wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor used in connection with the method(s) of the present invention is a compound that has the designation of COX 189 (B-211) and that has the structure shown in Formula (III) or an isomer, a pharmaceutically acceptable salt, ester, or prodrug thereof, wherein:

R¹⁶ is ethyl;

R¹⁷ and R¹⁹ are chloro;

R¹⁸ and R²⁰ are hydrogen; and

and R²¹ is methyl.

In connection with the present invention, drugs suitable for use as second drugs in combination with the COX 2 inhibitors include, but are not limited to, various categories of glutamate antagonists, γ-aminobutyric acid (GABA) receptor modulators, n-methyl-D-aspartate (NMDA) receptor antagonists and desensitizers, α-amino-3-hydroxy-5-methyl-4-isoxazole proprionate (AMPA) receptor antagonists and desensitizers, metabotropic receptor agonists and antagonists, glycine modulators, branched chain amino acids, neurotransmitter modulators, neuroprotective agents, neurotrophic growth factors, thyotrophin releasing hormone (TRH) modulators, apoptosis modulators, N-acetylated, alpha-linked acidic dipeptidase (NAALADase) inhibitors, caspase inhibitors, collagen synthesis modulators, oxidase inhibitors, nitric oxide synthase inhibitors, protein kinase inhibitors, mast cell activation inhibitors, glutathione synthase stimulants, VEGF antagonists, antioxidants, immunosuppressants, cytoskeletal proteins, andrenocortical steroids, drugs addressing various symptoms of ALS or offsetting side effects of ALS medications and therapeutic regimens, and drugs that otherwise treat ALS.

Examples of glutamate antagonists include, but are not limited to, riluzole (Rilutek®), Lamotrigine, dextromethorphan, EAAT2, Tamoxifen and topiramate (Topamax®).

Examples of γ-Aminobutyric Acid (GABA) Receptor Modulators include, but are not limited to, gabapentin (Neurontin®).

Examples of N-Methyl-D-Aspartate (NMDA) Receptor Antagonists and Desensitizers include, but are not limited to, 2-amino-5-phosphanovalerate (AP5), Eliprodil, 3-((+)-2-carboxypiperazin-4-yl)propyl-1-phosphonic acid (CPP), 1-(cis-2-carboxypiperazin-4-yl)propyl-1-phosphonic acid (selfotel or CGS 19755), D-(E)-4-(3-phosphonopro-2-2nyl) piperazine-2-carboxylic acid (CGP 37849), DL-(E)-2-amino-4-methyl-5-phosphono-3-pentenoic acid ethyl ester (CGP 39551), D-(E)-2-amino-4-methyl-5-phosphono-3-pentenoic acid (CGP 40116), 2R,4R,5S-(2-amino-4,5-(1,2-cyclohexyl)-7-phosphonoheptanoic acid) (NPC 17742), N-(1-Naphthyl)-N-(3-ethylphenyl)-N-methyl guanidine HCl (aptiganel/CNS 1102), (+)-3-methoxy-17-methylmorphinan (dextromethorphan), AVP-923 (dextromethorphan and enzyme inhibitor), (−)-1-methyl-1-phenyl-1-1,2,3,4-tetrahydroisoquinoline HCl (FR 115427), (−)-2-(2-chlorophenyl)-2-(methyl-amino) cyclohexanone (ketamine), 1-[4-(3-hydroxyphenyl)-1-methyl-4-piperidyl]-1-propanone (ketobemidone), 3,5-dimethyladamantan-1-amine (memantine), 6-dimethylamino-4,4-diphenyl-3-heptanone (methadone), 3-(2-carboxypiperizine-4-yl)-propenyl-1-phosphonate (dizocilpine or MK 801), 1-(1-phenecyclohexyl)piperidine (PCP) and 1-methyl-4-phenyl-4-piperidine carboxylic acid ethyl ester (pethidine or meperidine).

Examples of α-Amino-3-Hydroxy-5-Methyl-4-Isoxazole Proprionate (AMPA) Receptor Antagonists and Desensitizers include, but are not limited to, RPR-119990, LY-300164 or talampanel, 6-cyano-7-nitorquinoxaline-2,3-dione (CNQX), 6,7-dinitorquinoxaline-2,3-dione (DNQX), (3SR,4aRS,6RS,8aRS)-6-[2-(1H-tetrazol-5-yl)-ethyl]1,2,3,4,4a,5,6,7,8a-decahydroisoquinoline-3-carboxylic acid (LY 215490), 6-nitro-7-sulfamoylbenzo(f)-quinoxaline-2,3-dione (NNC 079202 or NBQX), 1,2,3,6,7,8-hexahydro-3-(hydroxyimino)-N,N-dimethyl-7-methyl-2-oxobenzo[2,1-b:3,4-C′]dipyrrole-5-solfonaminde (NS 257), 1-(4-aminophenyl)-4 methyl-7,8-methylenedeoxy-5H-2,3-benzodiazepine (GYKI 52466), 6-chloro-3,4-dihydro-3-(2-norbornen-5-yl)-7-sulfanol-1,2,4-benzothiadiazine 1,1-dioxide (cyclothiazide) and 7-chloro-3-methyl-3,4-dihydro-2H-1,2,4-benzothiadiazine s,s-dioxide (IDRA 21).

Examples of Metabotropic Receptor Agonists and Antagonists include, but are not limited to, (1S, 1′R, 2′R, 3′R)-2-(2,3-dicarboxycyclospropyl) glycine (DCG-IV), (2S, 1′S, 2′S)-2-(2-carboxycyclospropyl) glycine, 2-amino-4-phosphonobutyrate (AP4), trans-1-amino-cyclopentyl-1,3-dicarboxylate (t-ACPD), serine-O-phosphate (L-SOP), L-(+)-2-amino-3-phosphonoproprioionic acid (L-AP3), (S)-4-carboxy-3-hydroxy-phenylglycine (S-4C3HPG), (S)-4-carboxyphenylglycine (S-4CPG), (S)-2-amino-2-methyl-4-phosphonobutanoic acid (MAP-4) and (R,S)-α-methyl-4-carboxy-phenylglycine (RS-M4CPG).

Examples of Glycine Modulators include, but are not limited to, N-(3-[5-chloro-1-(4-chlorophenyl)indan-1-yl]propyl)-N-methylalanine.

Examples of Branched Chain Amino Acids include, but are not limited to, Valine, Leucine and Isoleucine.

Examples of Neurotransmitter Modulators include, but are not limited to, xaliproden or its hydrochloride salt (SR-57746A), T-588, 3,4 diaminopyridine, CPC-304, CPC-317, PD-176078 and cephalosporin ceftriaxone.

Examples of Neuroprotective Agents include, but are not limited to, glatiramer acetate (Copaxone®), huperzine A, 10-methyl-huperzine A, 10,10 dimethyl huperzine A, huperzine B, nicotine, epibaticline, cytosine, lobeline, anabasine, 7-chloro-5-methyl-1-[2-(4-methylpiperazin-1-yl)-2-oxoethyl]-4-oxo-3-phenyl-3,5-dihydro-4H-pyridazo[4,5-b]indole and 4-methyl-1-[7-chloro-3-(3-chlorophenyl)-5-methyl-4-oxo-3,5-dihydro-4H-pyridazino[4,5-b]indol-1-ylcarbonyl]piperazine.

Examples of Neurotrophic Growth Factors include, but are not limited to, ciliary neurotrophic factor (CNTF), brain derived neurotrophic factor (BNDF), recombinant insulin-like growth factor (rhIGF-1, Myotrophin® mecasermin, or Somatomedin C), vascular epithelial growth factor (VEGF), gilial cell derived neurotrophic factor (GDNF) or liatermin, neurturin, Pigment epithelium derived factor (PEDF), FKBO-neuroimmunophilin ligands, AIT-082, leteprinim potassium or NeotrofinT), buspirone, tetracycline, minocycline, doxycycline, emfilermin, cardiotrophin-1 (CT-1), NGF, neurotrophin-3 (NT-3), neurotrophin-4/5 (NT-4/5), EHT 201, EHT 202 and genistein.

Examples of Thyotrophin Releasing Hormone (TRH) Modulators include, but are not limited to, RX-77368, MK-771 and JTP-2942.

Examples of Apoptosis Modulators include, but are not limited to, TCH-346 and WHI-P131.

Examples of N-acetylated, Alpha-linked Acidic Dipeptidase Inhibitors (NAALADase Inhibitors) include, but are not limited to, GPI-5693 and GPI-5000.

Examples of Caspase Inhibitors include, but are not limited to, ZVAD fink.

Examples of Collagen Synthesis Modulators include, but are not limited to, 3-(2-phenyl-2-oxoethyl)-4,5-dimethylthiazolium salt.

Examples of Oxidase Inhibitors include, but are not limited to, nordihydroguaiaretic acid, para REV-5901 (L-655238), Bay-X-1005, ML-3000 and Zileuton.

Examples of Glutathione Synthase Stimulants include, but are not limited to, oxothiazolidine carboxylate (Procysteine®).

Examples of Vascular Endothelial Growth Factor (VEGF) Antagonists include, but are not limited to, ARR 17477.

Examples of Antioxidants include, but are not limited to, superoxide dismutase (SOD), recombinant human CuZn-SOD, glutathione, glutathione peroxidase, catalase, nitric oxide synthase, tocopherol (Vitamin E), ascorbic acid (Vitamin C), selenium, acetylcysteine, seleginine (Deprenyl®), pycnogenol, co-enzyme Q10, beta carotene, PC 01, SC-55858, edaravone, iron (III) porphyrins, mithramycin, chromomycin, daunomycin, olivomycin and WP-631.

Examples of Immunosuppressants include, but are not limited to, azathioprine, cyclophosphamide, cyclosporine, intravenous immune globulin (IVIG) and cytoxan.

Examples of Cytoskeletal Proteins Showing Abnormalities in ALS include, but are not limited to, metallothionine and apolipoprotein E.

Examples of Adrenocortical Steroids include, but are not limited to, oxandrolone, creatine, erythropeotin and dehydro epiandrosterone (DHEA).

Examples of antispastics include, but are not limited to, baclofen (Lioresal®), tizandidine (Zanaflex®), benzodiazepines, including clonazepam (Klonopin®), dantrolene (Dantrium®), dronabinol (Marinol®) and diazepam (Diastat®).

Examples of Drugs for Treating Emotional Lability (pseudobulbar effect) include, but are not limited to, AVP-923 (dextromethorphan hydrobromide and quinidine sulfate).

Examples of Drugs for Treating Respiratory Difficulties include, but are not limited to, morphine and lorazepam.

Examples of Drugs for Treating General Pain include, but are not limited to, aspirin, acetaminophen (Tylenol®), ibuprofen, oxycodone (Percocet®), or with aspirin (Percodan®) and tramadol (Ultram®).

Examples of Drugs for Treating Depression include, but are not limited to, amitriptylene, fluvoxamine (Luvox®), sertraline (Zoloft®) and fluoxetine (Prozac®).

Examples of Drugs for Treating Gastric Reflux include, but are not limited to, famotidine (Pepcid®) and ranitidine (Zantac®).

Examples of Drugs for Treating Excessive Salivation include, but are not limited to, glycopyrrolate (Robinul®), diphenhydramine hydrochloride (Benadryl®), copolamine, trihexyphenidyl, clonidine, propantheline or its bromide salt, tropine and botulinum toxin type A.

Examples of drugs for treating other indications include, but are not limited to, Robitussin®, docusate sodium (Colace®), tolterodine (Detrol®), TA-0910, ubiquinone, minocycline, indinavir, alpha lipoic acid, n-acetyl-cysteine (NAC), polyphenols, pregnenolone, threonine and methylcobalamin.

Without being bound by theory, the various categories of second drugs disclosed above, can be described generally according to the mechanisms by which they are currently believed to function. However, it is understood that the second drugs listed in Table 2 may function by an additional means, a combination of means, or in a manner different from that associated with the descriptions for their broader categories within which they are classified. The second drugs may also function according to mechanisms as yet unknown.

In describing the various categories set forth in Table 2, glutamate antagonists generally reduce the amount of glutamate in the brain by inhibiting its release or increasing the rate of its breakdown either directly or through the regulation of various enzymes or receptors involved in glutamate release or metabolism. The glutamate antagonist riluzole (Rilutek®) is currently the only FDA approved medication proven to prolong ALS patients' survival. Glutamate antagonists also include at least some alpha adrenoceptor agonists. In addition to those glutamate antagonists listed in Table 2, alkylnylamino acid derivatives described, for example, in EP 826663, are also included in this category. Modulators of brain glutamate transporters, such as antisense RNA, ribozymes, or catalytic DNA as described, for example, in WO 208384, are also included in this category.

GABA receptor modulators are those molecules having affinity for the gabapentin binding site of the GABA receptor, and therefore embrace GABA receptor agonists and antagonists. In addition to those GABA receptor modulators listed in Table 2, beta-thio-alpha-amino acids described, for example, in WO 00222568 are also included in this category.

NMDA and AMPA receptor antagonists and desensitizers are thought to be important in protecting neurons from glutamate neurotoxicity. In addition to those NMDA receptor antagonists listed in Table 2, 4-hydroxy-piperidine derivatives described, for example, in EP 824098, and arylsulfone derivatives described, for example, in WO 00216321, are also included in this category. In addition to those AMPA receptor antagonists and desensitizers listed in Table 2, quinoxalinedione compounds described, for example, in WO 9612724 and WO 9612725, and N-acylaminoacylpolyamine philanthotoxin analogs described, for example, in WO 00216314.

Without being bound by theory, it is believed that metabotropic receptor agonists and antagonists regulate complex interactions between metabotropic receptors and other glutamate receptors. In addition to those metabotropic receptor agonists and antagonists listed in Table 2, 5H-thiazolo[3,2-a]pyrimidine derivatives described, for example, in EP 891978, are also included in this category. Additionally included in this category are metabotropic glutamate receptor ligands, including bicyclic [3.1.0] hexanes and related compounds.

Without being bound by theory, it is believed that glycine modulators reduce or enhance the transport or uptake of glycine in the cells. Among other compounds, glycine modulators include those described in WO 00208216.

Without being bound by theory, it is believed that branched chain amino acids (BCAAs) play a role in the metabolism of glutamate.

Without being bound by theory, it is believed that neurotransmitter modulators function by altering neurochemical transmission at the neuromuscular junction. This category of drugs embraces both neurotransmitter agonists and antagonists, as well as acetylcholinesterase receptor modulators and ligands, and potassium, sodium, and calcium channel blockers. In addition to those neurotransmitter modulators listed in Table 2, heteroaryl diazabicycloalkanes described, for example, in WO 0144243; N-acetyl serotonin derivatives that modulate 5-HT receptors as described, for example, in U.S. Pat. No. 6,353,015; quinuclidine-substituted aryl compounds that act as alpha7 nicotinic acetylcholine receptor ligands, as described, for example, in WO-00216356; phenyl and phenyl-substituted ethanolamine and ethylene diamine compounds that act as sodium channel blockers, as described, for example, in WO-00216308; and protein-lipid complexes, proteases, protease inhibitors, intracellular proteins, receptor associated proteins, RAP memetics, and LRP-specific antibodies described, for example, in WO-00207755 are also included in this category.

Without being bound by theory, it is believed that neuroprotective agents attenuate or eliminate the causes or effects of physiological events that m a y damage and/or induce toxicity to neurons. In addition to those neuroprotective agents listed in Table 2, phenyl oxazoles, thiazoles, oxazolines, oxadiazoles, and benzoxazoles are also included in this category and may be hormone dependent or independent. Also within the category of neuroprotective agents are benzodiazepine receptor modulators that include pyridazine[4,5-b]indole-1-acetamide derivatives as described, for example, in WO-00207727 as well as 1-(4-oxo-3,5-dihydrido-4H-pyridzino[4,5-b]indol-1-ylcarbonyl) piperazine derivatives as described, for example, in WO-00208229. Huperzine alone or in combination with nicotinic compounds as described, for example, in WO-00211712, are also included in this category.

Without being bound by theory, it is believed that neurotrophic growth factors function by stimulating nerve cell generation or retarding cell death or otherwise stimulating or mimicking biological neurotrophic growth factors. Neurotrophic growth factors include the so-called “small molecule neurotrophic agents” thought to mimic various neurtrophic growth factors. Neurotrophic growth factors embrace nerve growth factor agonists, neural stem cells, insulin-like growth factor-1 agonists. In addition to the neurotrophic growth factors listed in Table 2, nerve growth promoting agents described, for example, in WO 9612717 are also included in this category.

Without being bound by theory, it is believed that thyotrophin releasing hormone (TRH) modulators generally act to enhance or decrease the effect of TRH, a tripeptide, in the anterior lobe of the pituitary, where it stimulates the release of thyroid stimulating hormone and prolactin.

Without being bound by theory, it is believed that apoptosis modulators generally increase cell life by regulating intracellular signals and/or the binding of so-called “death activators” (e.g., TNF-α, lymphotoxin, or Fas ligand). In addition to the apoptosis modulators listed in Table 2, polynucleotides encoding ICP10PK, as described, for example, in WO 00207776 are also included in this category.

Without being bound by theory, it is believed that NAALADase inhibitors provide protection against cellular damage by regulating excessive harmful levels of glutamate and/or by promoting the production of myelin, a protective sheath surrounding peripheral nerves. In addition to the NAALADase inhibitors listed in Table 2, phosphonic acid derivatives, as described, for example, in WO 9847906, are also included in this category.

Without being bound by theory, it is believed that caspase inhibitors refer to inhibitors of the family of cysteine, aspartyl-specific proteases that appear to be integral to the phenomenon of apoptosis in acute and chronic neurological disease.

Without being bound by theory, it is believed that collagen synthesis modulators function by inhibiting the formation of advanced glycosylation end product cross links between proteins and/or by the formation of bioactive agents such as growth factors and inflammatory mediators. Collagen synthesis modulators include substituted thiazolium, imidazolium, or oxazolium compounds or their salts as described in, for example, WO 00207725.

Without being bound by theory, it is believed that oxidase inhibitors function by inhibiting microglial activation. In addition to the oxidase inhibitors listed in Table 2, cyclooxidase, lipoxidase, and flap inhibitors as described, for example, in WO 00205825, are also included in this category.

Without being bound by theory, it is believed that glutathione synthase stimulants enhance the biosynthesis of glutathione, essential for a variety of cellular functions, from γ-glutamylcysteine and glycine.

Without being bound by theory, it is believed that vascular endothelial growth factor (VEGF) antagonists prevent the interaction of VEGF with its receptor, which may be beneficial for the treatment of a number of disorders. VEGF antagonists include fused pyrrolocarbazoles as described, for example, in WO-00217914.

Without being bound by theory, it is believed that nitric oxide (NO) synthase inhibitors help control the formation of NO, believed to be an intercellular messenger in central physiological mechanisms. NO is synthesized from L-arginine by nitric oxide synthase (NOS), as a response to activation of N-methyl-D-aspartate (NMDA) receptors by excitatory amino acids.

Without being bound by theory, it is believed that protein kinase inhibitors regulate the activity of protein kinases, which serve as predominant components of signal transduction pathways. Protein kinases useful in the method of the present invention include Janus Kinase 3 inhibitors and tyrosine kinase inhibitors. Compounds that may be used as protein kinase inhibitors include bis-N-substituted derivatives of staurosporine as described, for example, in WO 9613506.

Without being bound by theory, it is believed that mast cell activation inhibitors regulate the degranulation of mast cells and corresponding release of histamines and other chemical mediators after binding with the antibody IgE.

Without being bound by theory, it is believed that Interleukin-1 converting enzyme (ICE) inhibitors may control a critical step in cytokine maturation and signaling pathways, providing therapeutic benefits in the management of a number of chronic diseases.

Without being bound by theory, it is believed that antioxidants generally reduce the physiological tendency for the occurrence of potentially cell-damaging oxidation reactions. Antioxidants include enzymes involved in the metabolism of oxygen radicals (e.g. O²⁻) or the inhibitors of enzymes leading to the production of such radicals. Antioxidants also include free radical scavengers that may react directly with oxygen radicals. Also within the scope of free radical scavengers and antioxidants are chelating agents and chelated compounds that have the capacity to bind trace metals and metal ions, especially Zn, Cu, Mn, Mg, Cr, Se, that can catalyze or otherwise facilitate oxidation reactions. Such chelators include reduced forms of phenothiazines, riboflavin, ubiquinones, 4,7-phenanthroline-5,6-hydroquinone, or dapsone that are described, for example, in WO-00203972. More specific types of antioxidants of interest in the present invention include superoxide dismutase stimulants, oxidative stress eliminating or reducing agents as described, for example, in WO-00215912, and other reducing agents.

Without being bound by theory, it is believed that immunosuppressants generally can dampen the body's immune response, which includes the generation of antibodies that normally protect against infection.

Without being bound by theory, it is believed that cytoskeletal proteins include those found to be present in abnormal levels or forms in ALS patients, including various neurofilament proteins, copper handling proteins, excitory amino acid receptor proteins and transporters.

Without being bound by theory, it is believed that adrenocortical steroids generally interact with specific receptor proteins to regulate the expression of corticosteriod-responsive genes, thereby changing the levels and array of proteins synthesized. Drugs addressing various symptoms of ALS or offsetting side effects of ALS medications and therapeutic regimens include antispastics, drugs for the treatment of emotional lability, repiratory difficulties, general pain, depression, gastric reflux, excessive salivation, thick phlegm, constipation, urinary urgency, spinal function disorders, convulsions, various psychosocial problems, muscle atrophy, motor weakness, mobility difficulties, dysarthria (difficulty speaking) and dysphagia (difficulty swallowing). Other drugs for the treatment of ALS and/or its symptoms function by as yet unknown or undefined mechanisms.

Where polypeptides or polynucleotides, including enzymes, antibodies, vectors, cloning vehicles, gene delivery or gene therapy agents, are used as second drugs as described herein (e.g., superoxide dismutase), it is also meant that variants of such polypeptides or polynucleotides are also within the scope of the invention. Suitable variants of native or naturally occurring polypeptides or polynucleotides can also be fragments, analogs, and derivatives thereof. The term “fragment” is intended to include a polypeptide or polynucleotide consisting of only a part of the intact mature, human sequence and structure, and, in the case of a polypeptide, can be a C-terminal deletion or N-terminal deletion of the native polypeptide. The term “analog” is intended to include an analog of either the mature human polypeptide or polynucleotide or of a fragment thereof, where the analog comprises a native polypeptide or polynucleotide sequence and structure having one or more substitutions, insertions, or deletions. When referring to a polypeptide, “muteins” and polypeptides having one or more peptoids (peptide mimics) are also encompassed by the term analog (see International Publication No. WO 91/04282). The term “derivative” is intended to include any suitable modification of the native polypeptide or polynucleotide, of a fragment of the native polypeptide or polynucleotide, or of their respective analogs, such as, in the case of a polypeptide, a modification by glycosylation, phosphorylation, or other addition of foreign moieties, so long as the desired biological activity of the native polypeptide or polynucleotide is retained.

Certain subgroups of the above-noted second drugs, listed in Table 2, may be preferred for the treatment of ALS in combination with one or more of any of the Cox-2 inhibitors of Table 1. These subgroups include, but are not limited to, C-1 to C-5, C-6 to C-10, C-11 to C-15, C-16 to C-20, C-21 to C-25, C-26 to C-30, C-31 to C-35, C-36-C-40, C-41 to C-45, C-46 to C-50, C-51 to C-55, C-56 to C-60, C-61 to C-65, C-66 to C-70, C-71 to C-75, C-76 to C-80, C-81 to C-85, C—C-86 to C-90, C-91 to C-95, C-96 to C-100, C-101 to C-105, C-106 to C-l 10, C-111 to C-115, C-116 to C-120, C-121 to C-125, C-126 to C-130, C-131 to C-135, C-136 to C-140, C-141 to C-145, C-146 to C-150, C-151 to C-155, C-156 to C-160, C-161 to C-165, C-166 to C-170, C-171 to C-175, C-176 to C-181 or combinations thereof.

The cyclooxygenase-2 selective inhibitors described previously may be referred to herein collectively as COX 2 selective inhibitors, or cyclooxygenase-2 selective inhibitors.

Cyclooxygenase-2 selective inhibitors as well as second drugs that are useful in the present invention can be supplied by any source as long as the combination of drugs is pharmaceutically acceptable. Cyclooxygenase-2-selective inhibitors and second drugs can be isolated and purified from natural sources or can be synthesized. The combination of the cyclooxygenase-2-selective inhibitor(s) and second drug(s) should be of a quality and purity that is conventional in the trade for use in pharmaceutical products.

According to an embodiment of the present inventive method, a subject in need of treatment for ALS is administered an amount of at least one COX 2 selective inhibitor and an amount of at least one second drug, where the amount of the COX 2 selective inhibitor together with the amount of second drug(s) is a therapeutically effective amount sufficient to constitute an ALS treatment effective amount.

A pharmaceutical composition of one or more COX 2 inhibitors and one or more second drugs in connection with the method(s) of the present invention can be administered orally, for example, as tablets, coated tablets, dragees, troches, lozenges, gums, aqueous or oily suspensions, dispersible powders or granules, emulsions, hard or soft capsules, or syrups or elixirs. Compositions intended for oral use may be prepared according to any method known in the art for the-manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject pharmaceutical composition of COX 2 inhibitor(s) and second drug(s) in connection with the present inventive method can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject pharmaceutical composition of COX 2 inhibitor(s) and second drug(s) in connection with the present inventive method can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The pharmaceutical compositions of COX 2 inhibitor(s) and second drug(s) in connection with the present inventive method can also be administered topically, in the form of patches, creams, ointments, jellies, collyriums, solutions or suspensions. Of course, the compositions of the present invention can be administered by routes of administration other than topical administration. Also, as mentioned above, the COX 2 inhibitor(s) and second drug(s) may be administered separately, with each agent administered by any of the above mentioned administration routes. For example, the COX 2 inhibitor(s) may be administered orally in any or the above mentioned forms (e.g. in capsule form) while the second drug(s) is/are administered topically (e.g. as a cream).

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The following examples describe embodiments of the invention. Other embodiments within the scope of the embodiments herein will be apparent to one skilled in the art from consideration of the specification or practice of the invention as disclosed herein. It is intended that the specification, together with the examples, be considered to be exemplary only, with the scope and spirit of the invention being indicated by the embodiments and the examples. In the examples, all percentages are given on a weight basis unless otherwise indicated.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in this application shall be interpreted as illustrative and not in a limiting sense. Exemplary non-limiting embodiments of the present invention are provided below.

Exemplary ALS symptoms that may be treated with the compositions of Tables 1-1A above, in combination with the compositions of Table 2 above, are indicated in Table 5 below:;

TABLE 5

| | Exemplary ALS Indication(s) treated with the |
|---|---|
| No. | COX 2-specific inhibitor of Tables 1-1A |
| 1. | Spasticity |
| 2. | Emotional Lability |
| 3. | Respiratory Difficulties |
| 4. | General Pain |
| 5. | Depression |
| 6. | Gastric Reflux |
| 7. | Excessive Salivation |
| 8. | Thick Phlegm |
| 9. | Constipation |
| 10. | Urinary Urgency |
| 11. | Various Psychosocial Problems |
| 12. | Muscle Atrophy |
| 13. | Motor Weakness |
| 14. | Mobility Difficulties |
| 15. | Dysarthria |
| 16. | Dysphagia |
| 17. | Fatigue |
| 18. | Clumsiness |
| 19. | Behavior Changes |
| 20. | Psychiatric Manifestations |
| 21. | Slurred Speech |
| 22. | Spinal Function Disorders |
| 23. | Convulsions |

The following Tables 6 and 7 list various dosage forms of the pharmaceutical composition for use in conjunction with the method of the present invention. Note that the dosage forms in Table 7 exclude all dosage forms that may be transdermally applied. By contrast, Table 6 includes such transdermally applied dosage forms.

TABLE 6

| | |
|---|---|
| | Exemplary Dosage Forms (other than those |
| No. | that are transdermally applied) |

Oral dosage forms

| | |
|---|---|
| 1. | Tablet |
| 2. | Slow Release Tablet |
| 3. | Effervescent Tablet |
| 4. | Enteric Coated Tablet |
| 5. | Compressed Tablet |
| 6. | Molded Tablet |
| 7. | Capsule |
| 8. | Slow Release Capsule |
| 9. | Capsule for Use in or with Nebulizer |
| 10. | Gelatin Capsule |
| 11. | Caplet |
| 12. | Troche |
| 13. | Powder |
| 14. | Lozenge |
| 15. | Gum |
| 16. | Solution |
| 17. | Suspension |
| 18. | Emulsion |
| 19. | Dispersion |

Parenteral Dosage Forms

| | |
|---|---|
| 20. | Intramuscular Injection |
| 21. | Intravenous Injection |
| 22. | Inhalant |
| 23. | Aerosol |
| 24. | Nebulizing Liquid |
| 25. | Elixir |
| 26. | Collyria |
| 27. | Injection |
| 28. | Pellets |
| 29. | Implants |
| 30. | Otic Solution |
| 31. | Suppository |
| 32. | Syrup |
| 33. | Tincture |
| 34. | Opthalmic Solution |
| 35. | Oral Gel |
| 36. | Oral Paste |
| 37. | Oral Inhalant |

TABLE 7

| No. | Exemplary dosage Forms (that are topically applied) |
|---|---|
| 1. | Liquid |
| 2. | Emulsion |
| 3. | Dispersion |
| 4. | Gel |
| 5. | Paste |
| 6. | Cream |
| 7. | Lotion |
| 8. | Extract |
| 9. | Ointment |
| 10. | Patch |
| 11. | Implant |
| 12. | Pellet |
| 13. | Topical Powder |
| 14. | Topical Solution |

For a more complete list of dosage forms in addition to those provided in Tables 4 and 5, see Remington's Pharmaceutical Sciences, Mack Publishing Co., Easton, Pa., Arthur Osol (editor), 16^{th} Edition (1980). Also see each of the later editions of the same (i.e., each later edition to date of Remington's Pharmaceutical Sciences). Also see, The United States Pharmacopeia, 21^{st} Edition, United States Pharmacopeial Convention, Washington, D.C. (1985). Also see each of the later editions of the same (i.e., each later edition to date of The United States Pharmacopeia).

What is claimed is:

1. A method for treating amyotrophic lateral sclerosis (ALS) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (I): 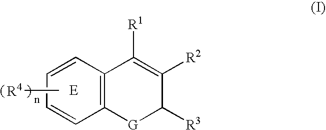
or an isomer, a pharmaceutically acceptable salt, ester, or prodrug thereof;
wherein:
G is O, S or NR^{a};
R^{a} is alkyl;
R¹ is H or aryl;
R² is carboxyl, aminocarbonyl, alkylsulfonylaminocarbonyl or alkoxycarbonyl;
R³ is haloalkyl, alkyl, aralkyl, cycloalkyl or aryl optionally and independently substituted with one or more radicals selected from alkylthio, nitro and alkylsulfonyl;
n is an integer which is 1, 2, 3, or 4; and
each R⁴ is independently H, halo, alkyl, aryl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, mono- or dialkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, aralkylcarbonyl, heteroarylcarbonyl, arylcarbonyl, aminocarbonyl, alkylcarbonyl, aryl, or heteroaryl;
wherein said aryl and heteroaryl radicals are optionally and independently substituted with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
or wherein R⁴ together with the atoms to which R⁴ is attached and the remainder of ring E forms a naphthyl radical.

2. The method of claim, 1 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C—C-101, C-102, C-103, C-104, C-105, C-106, C-107, C-108, C-109, C-110, C-111, C-112, C-113, C-114, C-115, C-116, C-117, C-118, C-119, C-120, C-121, C-122, C-123, C-124, C-125, C-126, C-127, C-128, C-129, C-130, C-131, C-132, C-133, C-134, C-135, C-136, C-137, C-138, C-139, C-140, C-141, C-142, C-143, C-144, C-145, C-146, C-147, C-148, C-149, C-150, C-151, C-152, C-153, C-154, C-155, C-156, C-157, C-158, C-159, C-160, C-161, C-162, C-163, C-164, C-165, C-166, C-167, C-168, C-169, C-170, C-171, C-172, C-173, C-174, C-175, C-176, C-177, C-178, C-179, C-180, or an isomer, a pharmaceutically acceptable salt, ester, or prodrug thereof.

3. The method of claim 1 wherein said second drug is a glutamate antagonist, a neurotrophic growth factor, an antioxidant, an immunosuppressant, a cytoskeletal protein, an adrenocortical steroid, or an anti spastic.

4. The method of claim 1, wherein:
G is O or S;
R² is carboxyl, lower alkyl, lower aralkyl and lower alkoxycarbonyl;
R³ is lower haloalkyl, lower cycloalkyl and phenyl; and
each of one or more R⁴ is independently H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, lower aralkylcarbonyl, lower alkylcarbonyl, and phenyl optionally and independently substituted with one or more radicals selected from the group consisting of alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
or wherein R⁴ together with the atoms to which R⁴ is attached and the remainder of ring E forms a naphthyl radical.

5. The method of claim 4, wherein:
R² is carboxyl;
R³ is lower haloalkyl; and
each of one or more R⁴ is independently H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl;
or wherein R⁴ together with the atoms to which R⁴ is attached and the remainder of ring E forms a naphthyl radical.

6. The method of claim 5, wherein:
said lower haloalkyl R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each or one or more R⁴ is independently H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, benzylaminosulfonyl, N-ethylsulfonyl, 2,2-dimethylethylaminosulfonyl, N,N-dimethylaminosulfonyl, isopropylaminosulfonyl, N-(2-methylpropyl)aminosulfonyl, N-morpholinosulfonyl, methylsulfonyl, benzylcarbonyl, 2,2-dimethylpropylcarbonyl, phenylacetyl, or phenyl;
or wherein R⁴ together with the atoms to which R⁴ is attached and the remainder of the ring E forms a naphthyl radical.

7. The method of claim 6, wherein:
R³is trifluoromethyl or pentafluoroethyl; and
each of one or more R⁴ is independently H, chloro, fluoro, bromo, iodo, methyl, ethyl, isopropyl, tert-butyl, methoxy, trifluoromethyl, trifluoromethoxy, N,N-diethylamino, N-phenylmethylaminosulfonyl, N-phenylethylaminosulfonyl, N-(2-furylmethyl)aminosulfonyl, N,N-dimethylaminosulfonyl, N-methylaminosulfonyl, benzylaminosulfonyl, N-(2,2-dimethylethyl)aminosulfonyl, isopropylaminosulfonyl, dimethylaminosulfonyl, 2-methylpropylaminosulfonyl, N-morpholinosulfonyl, methylsulfonyl, benzylcarbonyl, or phenyl;
or wherein R⁴ together with the atoms to which R⁴ is attached and the remainder of ring E forms a naphthyl radical.

8. The method of claim 7, wherein:
R³ is trifluoromethyl or pentafluoroethyl;
each of one or more R⁴ is independently H, methyl, ethyl, isopropyl, tert-butyl, chloro, bromo, fluoro, iodo, methyl, tert-butyl, trifluoromethoxy, methoxy, benzylcarbonyl, dimethylaminosulfonyl, isopropylaminosulfonyl, N-methylaminosulfonyl, benzylaminosulfonyl, phenylethylaminosulfonyl, methylpropylaminosulfonyl, methylsulfonyl, morpholinosulfonyl, N,N-diethylamino, or phenyl.

9. The method of claim 1, wherein said COX 2 inhibitor or isomer, pharmaceutically acceptable salt, ester, or prodrug thereof has a COX 2 IC₅₀ of less than about 5 μmol/L.

10. The method of claim 1, wherein said COX 2 inhibitor or isomer, pharmaceutically acceptable salt, ester, or prodrug thereof has a selectivity ratio of COX 1 IC₅₀ to COX 2 IC₅₀ of at least about 10.

11. The method of claim 10, wherein said COX 2 inhibitor or isomer, pharmaceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

12. The method of claim 1, wherein said COX 2 inhibitor or isomer, pharmaceutically acceptable salt, ester, or prodrug thereof has a COX 1 IC₅₀ of at least about 1 μmol/L.

13. The method of claim 12, wherein said COX 2 inhibitor or isomer, pharmaceutically acceptable salt, ester, or prodrug thereof has a COX 1 IC₅₀ of at least about 20 μmol/L.

14. The method of claim 1, wherein said first amount is from about 0.001 to about 100 mg/day per kg of body weight of said subject and said second amount is from about 0.001 to about 100 mg/day per kg of body weight of said subject.

15. The method of claim 14, wherein said first amount is from about 0.05 to about 50 mg/day per kg of body weight of said subject and said second amount is from about 0.1 to about 10 mg/day per kg of body weight of said subject.

16. The method of claim 15, wherein said first amount is from about 1 to about 20 mg/day per kg of body weight of said subject and said second amount is from about 0.5 to about 2 mg/day per kg of body weight of said subject.

17. The method of claim 1 wherein a weight ratio of said first amount to said second amount is from about 0.002 to about 10.

18. The method of claim 17 wherein a weight ratio of said first amount to said second amount is from about 0.1 to about 5.

19. The method of claim 1, wherein said subject is an animal.

20. The method of claim 19, wherein said subject is a human.

21. The method of claim 1, wherein said COX 2 inhibitor or isomer, pharmaceutically acceptable salt, ester, or prodrug thereof and said second drug are administered enterally or parenterally in one or more doses per day.

22. The method of claim 1, wherein said COX 2 inhibitor or isomer, pharmaceutically acceptable salt, ester, or prodrug thereof and said second drug are administered substantially simultaneously.

23. The method of claim 1, wherein said COX 2 inhibitor or isomer, pharmaceutically acceptable salt, ester, or prodrug thereof and said second drug are administered sequentially.

24. A method for treating amyotrophic lateral sclerosis (ALS) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (II): 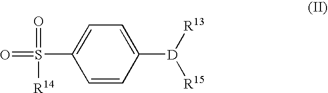
or an isomer, a pharmaceutically acceptable salt, ester, or prodrug thereof, provided that Formula (II) is not celecoxib (B-18) or rofecoxib (B-21),
wherein:
D is a partially unsaturated or saturated heterocyclyl ring or a partially unsaturated or saturated carbocyclic ring;
R¹³ is heterocyclyl, cycloalkyl, cycloalkenyl and aryl, wherein R¹³ is optionally substituted at a substitutable position with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
R¹⁴ is methyl or amino; and
R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

25. A method for treating amyotrophic lateral sclerosis (ALS) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (II): 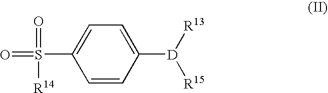
or an isomer, a pharmaceutically acceptable salt, ester, or prodrug thereof,
wherein:
D is a partially unsaturated or saturated heterocyclyl ring or a partially unsaturated or saturated carbocyclic ring;
R¹³ is heterocyclyl, cycloalkyl, cycloalkenyl and aryl, wherein R¹³ is optionally substituted at a substitutable position with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
R¹⁴ is methyl or amino; and
R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl; and,
wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C—C-101, C-102, C-103, C-104, C-105, C-106, C-107, C-108, C-109, C-110, C-111, C-112, C-113, C-114, C-115, C-116, C-117, C-118, C-119, C-120, C-121, C-122, C-123, C-124, C-125, C-126, C-127, C-128, C-129, C-130, C-131, C-132, C-133, C-134, C-135, C-136, C-137, C-138, C-139, C-140, C-141, C-142, C-143, C-144, C-145, C-146, C-147, C-148, C-149, C-150, C-151, C-152, C-153, C-154, C-155, C-156, C-157, C-158, C-159, C-160, C-161, C-162, C-163, C-164, C-165, C-166, C-167, C-168, C-169, C-170, C-171, C-172, C-173, C-174, C-175, C-176, C-177, C-178, C-179, C-180, or an isomer, pharmaceutically acceptable salt, ester, or prodrug thereof.

26. A method for treating amyotrophic lateral sclerosis (ALS) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (II): 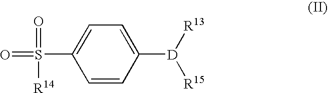
or an isomer, a pharmaceutically acceptable salt, ester, or prodrug thereof,
wherein:
D is a partially unsaturated or saturated heterocyclyl ring or a partially unsaturated or saturated carbocyclic ring;
R¹³ is heterocyclyl, cycloalkyl, cycloalkenyl and aryl, wherein R¹³ is optionally substituted at a substitutable position with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
R¹⁴ is methyl or amino; and
R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl; and,
wherein said second drug is a glutamate antagonist, a neurotrophic growth factor, an antioxidant, an immunosuppressant, a cytoskeletal protein, an adrenocortical steroid, or an antispastic.

27. The method of claim 24, wherein said COX 2 inhibitor or isomer, pharmaceutically acceptable salt, ester, or prodrug thereof has a COX 2 IC₅₀ of less than about 5 μmol/L.

28. The method of claim 24, wherein said COX 2 inhibitor or isomer, pharmaceutically acceptable salt, ester, or prodrug thereof has a selectivity ratio of COX 1 IC₅₀ to COX 2 IC₅₀ of at least about 10.

29. The method of claim 28, wherein said COX 2 inhibitor or isomer, pharmaceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

30. The method of claim 24, wherein said COX 2 inhibitor or isomer, pharmaceutically acceptable salt, ester, or prodrug thereof has a COX 1 IC₅₀ of at least about 1 μmol/L.

31. The method of claim 30, wherein said COX 2 inhibitor or isomer, pharmaceutically acceptable salt, ester, or prodrug thereof has a COX 1 IC₅₀ of at least about 20 μmol/L.

32. The method of claim 24, wherein said first amount is from about 0.001 to about 100 mg/day per kg of body weight of said subject and said second amount is from about 0.001 to about 100 mg/day per kg of body weight of said subject.

33. The method of claim 32, wherein said first amount is from about 0.05 to about 50 mg/day per kg of body weight of said subject and said second amount is from about 0.1 to about 10 mg/day per kg of body weight of said subject.

34. The method of claim 33, wherein said first amount is from about 1 to about 20 mg/day per kg of body weight of said subject and said second amount is from about 0.5 to about 2 mg/day per kg of body weight of said subject.

35. The method of claim 24 wherein a weight ratio of said first amount to said second amount is from about 0.002 to about 10.

36. The method of claim 24 wherein a weight ratio of said first amount to said second amount is from about 0.1 to about 5.

37. The method of claim 24, wherein said subject is an animal.

38. The method of claim 37, wherein said subject is a human.

39. The method of claim 24, wherein said COX 2 inhibitor or isomer, pharmaceutically acceptable salt, ester, or prodrug thereof and said second drug are administered enterally or parenterally in one or more doses per day.

40. The method of claim 24, wherein said COX 2 inhibitor or isomer, pharmaceutically acceptable salt, ester, or prodrug thereof and said second drug are administered substantially simultaneously.

41. The method of claim 24, wherein said COX 2 inhibitor or isomer, pharmaceutically acceptable salt, ester, or prodrug thereof and said second drug are administered sequentially.

42. A method for treating amyotrophic lateral sclerosis (ALS) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (III): 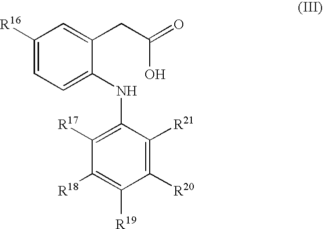
or an isomer, a pharmaceutically acceptable salt, ester, or prodrug thereof, wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H.

43. The method of claim 42 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C—C-101, C-102, C-103, C-104, C-105, C-106, C-107, C-108, C-109, C-110, C-111, C-112, C-113, C-114, C-115, C-116, C-117, C-118, C-119, C-120, C-121, C-122, C-123, C-124, C-125, C-126, C-127, C-128, C-129, C-130, C-131, C-132, C-133, C-134, C-135, C-136, C-137, C-138, C-139, C-140, C-141, C-142, C-143, C-144, C-145, C-146, C-147, C-148, C-149, C-150, C-151, C-152, C-153, C-154, C-155, C-156, C-157, C-158, C-159, C-160, C-161, C-162, C-163, C-164, C-165, C-166, C-167, C-168, C-169, C-170, C-171, C-172, C-173, C-174, C-175, C-176, C-177, C-178, C-179, C-180, or an isomer, a pharmaceutically acceptable salt, ester, or prodrug thereof.

44. The method of claim 42 wherein said second drug is a glutamate antagonist, a neurotrophic growth factor, an antioxidant, an immunosuppressant, a cytoskeletal protein, an adrenocortical steroid, or an anti spastic.

45. The method of claim 42, wherein:
R¹⁶ is ethyl; R¹⁷ and R¹⁹ are chloro; R¹⁸ and R²⁰ are hydrogen; and R²¹ is methyl.

46. The method of claim 42, wherein said COX 2 inhibitor or isomer, pharmaceutically acceptable salt, ester, or prodrug thereof has a COX 2 IC₅₀ of less than about 5 μmol/L.

47. The method of claim 42, wherein said COX 2 inhibitor or isomer, pharmaceutically acceptable salt, ester, or prodrug thereof has a selectivity ratio of COX 1 IC₅₀ to COX 2 IC₅₀ of at least about 10.

48. The method of claim 47, wherein said COX 2 inhibitor or isomer, pharmaceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

49. The method of claim 42, wherein said COX 2 inhibitor or isomer, pharmaceutically acceptable salt, ester, or prodrug thereof has a COX 1 IC₅₀ of at least about 1 μmol/L.

50. The method of claim 49, wherein said COX 2 inhibitor or isomer, pharmaceutically acceptable salt, ester, or prodrug thereof has a COX 1 IC₅₀ of at least about 20 μmol/L.

51. The method of claim 42, wherein said first amount is from about 0.001 to about 100 mg/day per kg of body weight of said subject and said second amount is from about 0.001 to about 100 mg/day per kg of body weight of said subject.

52. The method of claim 51, wherein said first amount is from about 0.05 to about 50 mg/day per kg of body weight of said subject and said second amount is from about 0.1 to about 10 mg/day per kg of body weight of said subject.

53. The method of claim 52, wherein said first amount is from about 1 to about 20 mg/day per kg of body weight of said subject and said second amount is from about 0.5 to about 2 mg/day per kg of body weight of said subject.

54. The method of claim 42 wherein a weight ratio of said first amount to said second amount is from about 0.002 to about 10.

55. The method of claim 54 wherein a weight ratio of said first amount to said second amount is from about 0.1 to about 5.

56. The method of claim 42, wherein said subject is an animal.

57. The method of claim 56, wherein said subject is a human.

58. The method of claim 42, wherein said COX 2 inhibitor or isomer, pharmaceutically acceptable salt, ester, or prodrug thereof and said second drug are administered enterally or parenterally in one or more doses per day.

59. The method of claim 42, wherein said COX 2 inhibitor or isomer, pharmaceutically acceptable salt, ester, or prodrug thereof and said second drug are administered substantially simultaneously.

60. The method of claim 42, wherein said COX 2 inhibitor or isomer, pharmaceutically acceptable salt, ester, or prodrug thereof and said second drug are administered sequentially.

61. A method for treating amyotrophic lateral sclerosis (ALS) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (WV): 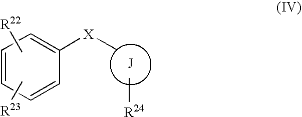
or an isomer, a pharmaceutically acceptable salt, ester, or prodrug thereof, wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

62. The method of claim 61 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C—C-101, C-102, C-103, C-104, C-105, C-106, C-107, C-108, C-109, C-110, C-111, C-112, C-113, C-114, C-115, C-116, C-117, C-118, C-119, C-120, C-121, C-122, C-123, C-124, C-125, C-126, C-127, C-128, C-129, C-130, C-131, C-132, C-133, C-134, C-135, C-136, C-137, C-138, C-139, C-140, C-141, C-142, C-143, C-144, C-145, C-146, C-147, C-148, C-149, C-150, C-151, C-152, C-153, C-154, C-155, C-156, C-157, C-158, C-159, C-160, C-161, C-162, C-163, C-164, C-165, C-166, C-167, C-168, C-169, C-170, C-171, C-172, C-173, C-174, C-175, C-176, C-177, C-178, C-179, C-180, or an isomer, a pharmaceutically acceptable salt, ester, or prodrug thereof.

63. The method of claim 62 wherein said second drug is a glutamate antagonist, a neurotrophic growth factor, an antioxidant, an immunosuppressant, a cytoskeletal protein, an adrenocortical steroid, or an anti spastic.

64. The method of claim 61 wherein said COX 2 inhibitor is nimesulide (B-212), flosulide (B-213), NS-398 (B-26), L-745337 (B-214), RWJ-63556 (B-215), or L-784512 (B-216).

65. The method of claim 61, wherein said COX 2 inhibitor or isomer, pharmaceutically acceptable salt, ester, or prodrug thereof has a COX 2 IC₅₀ of less than about 5 μmol/L.

66. The method of claim 61, wherein said COX 2 inhibitor or isomer, pharmaceutically acceptable salt, ester, or prodrug thereof has a selectivity ratio of COX 1 IC₅₀ to COX 2 IC₅₀ of at least about 10.

67. The method of claim 66, wherein said COX 2 inhibitor or isomer, pharmaceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

68. The method of claim 61, wherein said COX 2 inhibitor or isomer, pharmaceutically acceptable salt, ester, or prodrug thereof has a COX 1 IC₅₀ of at least about 1 μmol/L.

69. The method of claim 68, wherein said COX 2 inhibitor or isomer, pharmaceutically acceptable salt, ester, or prodrug thereof has a COX 1 IC₅₀ of at least about 20 μmol/L.

70. The method of claim 61, wherein said first amount is from about 0.001 to about 100 mg/day per kg of body weight of said subject and said second amount is from about 0.001 to about 100 mg/day per kg of body weight of said subject.

71. The method of claim 70, wherein said first amount is from about 0.05 to about 50 mg/day per kg of body weight of said subject and said second amount is from about 0.1 to about 10 mg/day per kg of body weight of said subject.

72. The method of claim 71, wherein said first amount is from about 1 to about 20 mg/day per kg of body weight of said subject and said second amount is from about 0.5 to about 2 mg/day per kg of body weight of said subject.

73. The method of claim 61 wherein a weight ratio of said first amount to said second amount is from about 0.002 to about 10.

74. The method of claim 73 wherein a weight ratio of said first amount to said second amount is from about 0.1 to about 5.

75. The method of claim 61, wherein said subject is an animal.

76. The method of claim 75, wherein said subject is a human.

77. The method of claim 61, wherein said COX 2 inhibitor or isomer, pharmaceutically acceptable salt, ester, or prodrug thereof and said second drug are administered enterally or parenterally in one or more doses per day.

78. The method of claim 61, wherein said COX 2 inhibitor or isomer, pharmaceutically acceptable salt, ester, or prodrug thereof and said second drug are administered substantially simultaneously.

79. The method of claim 61, wherein said COX 2 inhibitor or isomer, pharmaceutically acceptable salt, ester, or prodrug thereof and said second drug are administered sequentially.

80. A method for treating amyotrophic lateral sclerosis (ALS) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (V): 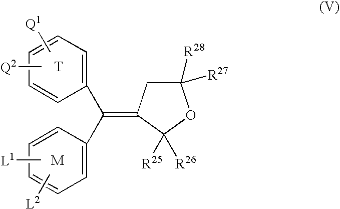
or an isomer, pharmaceutically acceptable salt, ester, or prodrug thereof, wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms, a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

81. The method of claim 80 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C—C-101, C-102, C-103, C-104, C-105, C-106, C-107, C-108, C-109, C-110, C-111, C-112, C-113, C-114, C-115, C-116, C-117, C-118, C-119, C-120, C-121, C-122, C-123, C-124, C-125, C-126, C-127, C-128, C-129, C-130, C-131, C-132, C-133, C-134, C-135, C-136, C-137, C-138, C-139, C-140, C-141, C-142, C-143, C-144, C-145, C-146, C-147, C-148, C-149, C-150, C-151, C-152, C-153, C-154, C-155, C-156, C-157, C-158, C-159, C-160, C-161, C-162, C-163, C-164, C-165, C-166, C-167, C-168, C-169, C-170, C-171, C-172, C-173, C-174, C-175, C-176, C-177, C-178, C-179, C-180, or an isomer, a pharmaceutically acceptable salt, ester, or prodrug thereof.

82. The method of claim 80 wherein said second drug is a glutamate antagonist, a neurotrophic growth factor, an antioxidant, an immunosuppressant, a cytoskeletal protein, an adrenocortical steroid, or an antispastic.

83. The method of claim 80 wherein said COX 2 inhibitor is N-(2-cyclohexyloxynitrophenyl)methane sulfonamide, or (E)-4-[(4-methylphenyl)(tetrahydro-2-oxo-3-furanylidene) methyl]benzenesulfonamide.

84. The method of claim 80, wherein said COX 2 inhibitor or isomer, pharmaceutically acceptable salt, ester, or prodrug thereof has a COX 2 IC₅₀ of less than about 5 μmol/L.

85. The method of claim 80, wherein said COX 2 inhibitor or isomer, pharmaceutically acceptable salt, ester, or prodrug thereof has a selectivity ratio of COX 1 IC₅₀ to COX 2 IC₅₀ of at least about 10.

86. The method of claim 85, wherein said COX 2 inhibitor or isomer, pharmaceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

87. The method of claim 80, wherein said COX 2 inhibitor or isomer, pharmaceutically acceptable salt, ester, or prodrug thereof has a COX 1 IC₅₀ of at least about 1 μmol/L.

88. The method of claim 87, wherein said COX 2 inhibitor or isomer, pharmaceutically acceptable salt, ester, or prodrug thereof has a COX 1 IC₅₀ of at least about 20 μmol/L.

89. The method of claim 80, wherein said first amount is from about 0.001 to about 100 mg/day per kg of body weight of said subject and said second amount is from about 0.001 to about 100 mg/day per kg of body weight of said subject.

90. The method of claim 89, wherein said first amount is from about 0.05 to about 50 mg/day per kg of body weight of said subject and said second amount is from about 0.1 to about 10 mg/day per kg of body weight of said subject.

91. The method of claim 90, wherein said first amount is from about 1 to about 20 mg/day per kg of body weight of said subject and said second amount is from about 0.5 to about 2 mg/day per kg of body weight of said subject.

92. The method of claim 80 wherein a weight ratio of said first amount to said second amount is from about 0.002 to about 10.

93. The method of claim 92 wherein a weight ratio of said first amount to said second amount is from about 0.1 to about 5.

94. The method of claim 80, wherein said subject is an animal.

95. The method of claim 94, wherein said subject is a human.

96. The method of claim 80, wherein said COX 2 inhibitor or isomer, pharmaceutically acceptable salt, ester, or prodrug thereof and said second drug are administered enterally or parenterally in one or more doses per day.

97. The method of claim 80, wherein said COX 2 inhibitor or isomer, pharmaceutically acceptable salt, ester, or prodrug thereof and said second drug are administered substantially simultaneously.

98. The method of claim 80, wherein said COX 2 inhibitor or isomer, pharmaceutically acceptable salt, ester, or prodrug thereof and said second drug are administered sequentially.

99. A method for treating amyotrophic lateral sclerosis (ALS) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor comprises B-1, B-2, B-3, B-4, B-5, B-6, B-7, B-8, B-9, B-10, B-11, B-12, B-13, B-14, B-15, B-16, B-17, B-19, B-20, B-22, B-23, B-24, B-25, B-26, B-27, B-28, B-29, B-30, B-31, B-32, B-33, B-34, B-35, B-36, B-37, B-38, B-39, B-40, B-41, B-42, B-43, B-44, B-45, B-46, B-47, B-48, B-49, B-50, B-51, B-52, B-53, B-54, B-55, B-56, B-57, B-58, B-59, B-60, B-61, B-62, B-63, B-64, B-65, B-66, B-67, B-68, B-69, B-70, B-71, B-72, B-73, B-74, B-75, B-76, B-77, B-78, B-79, B-80, B-81, B-82, B-83, B-84, B-85, B-86, B-87, B-88, B-89, B-90, B-91, B-92, B-93, B-94, B-95, B-96, B-97, B-98, B-99, B-100, B-101, B-102, B-103, B-104, B-105, B-106, B-107, B-108, B-109, B-110, B-111, B-112, B-113, B-114, B-115, B-116, B-117, B-118, B-119, B-120, B-121, B-122, B-123, B-124, B-125, B-126, B-127, B-128, B-129, B-130, B-131, B-132, B-133, B-134, B-135, B-136, B-137, B-138, B-139, B-140, B-141, B-142, B-143, B-144, B-145, B-146, B-147, B-148, B-149, B-150, B-151, B-152, B-153, B-154, B-155, B-156, B-157, B-158, B-159, B-160, B-161, B-162, B-163, B-164, B-165, B-166, B-167, B-168, B-169, B-170, B-171, B-172, B-173, B-174, B-175, B-176, B-177, B-178, B-179, B-180, B-181, B-182, B-183, B-184, B-185, B-186, B-187, B-188, B-189, B-190, B-191, B-192, B-193, B-194, B-195, B-196, B-197, B-198, B-199, B-200, B-201, B-202, B-203, B-204, B-205, B-206, B-207, B-208, B-209, B-210, B-211, B-212, B-213, B-214, B-215, B-216, B-217, B-218, B-219, B-220, B-221, B-222, B-223, B-224, B-225, B-226, B-227, B-228, B-229, B-230, B-231, B-232, B-233 or an isomer, a pharmaceutically acceptable salt, ester, or prodrug thereof.

100. The method of claim 99 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, C-83, C-84, C-85, C-86, C-87, C-88, C-89, C-90, C-91, C-92, C-93, C-94, C-95, C-96, C-97, C-98, C-99, C—C-101, C-102, C-103, C-104, C-105, C-106, C-107, C-108, C-109, C-110, C-111, C-112, C-113, C-114, C-115, C-116, C-117, C-118, C-119, C-120, C-121, C-122, C-123, C-124, C-125, C-126, C-127, C-128, C-129, C-130, C-131, C-132, C-133, C-134, C-135, C-136, C-137, C-138, C-139, C-140, C-141, C-142, C-143, C-144, C-145, C-146, C-147, C-148, C-149, C-150, C-151, C-152, C-153, C-154, C-155, C-156, C-157, C-158, C-159, C-160, C-161, C-162, C-163, C-164, C-165, C-166, C-167, C-168, C-169, C-170, C-171, C-172, C-173, C-174, C-175, C-176, C-177, C-178, C-179, C-180, or an isomer, a pharmaceutically acceptable salt, ester, or prodrug thereof.

101. The method of claim 99 wherein said second drug is a glutamate antagonist, a neurotrophic growth factor, an antioxidant, an immunosuppressant, a cytoskeletal protein, an adrenocortical steroid, or an anti spastic.

102. The method of claim 99 wherein said COX 2 inhibitor is valdecoxib (B-19), deracoxib (B-20), etoricoxib (B-22), JTE-522 (B-23), parecoxib (B-24), ABT-963 (B-25), or BMS-347070 (B-74), and an isomer, a pharmaceutically acceptable salt, ester, or prodrug thereof.

103. The method of claim 102 wherein said COX 2 inhibitor is etoricoxib (B-22), JTE-522 (B-23), parecoxib (B-24), ABT-963 (B-25), or BMS-347070 (B-74).

104. The method of claim 103, wherein said COX 2 inhibitor is sodium parecoxib.

105. The method of claim 99, wherein said COX 2 inhibitor, isomer, pharmaceutically acceptable salt, ester, or prodrug thereof has a COX 2 IC₅₀ of less than about 5 μmol/L.

106. The method of claim 99, wherein said COX 2 inhibitor, isomer, pharmaceutically acceptable salt, ester, or prodrug thereof has a selectivity ratio of COX 1 IC₅₀ to COX 2 IC₅₀ of at least about 10.

107. The method of claim 106, wherein said COX 2 inhibitor, isomer, pharmaceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

108. The method of claim 99, wherein said COX 2 inhibitor, isomer, pharmaceutically acceptable salt, ester, or prodrug thereof has a COX 1 IC₅₀ of at least about 1 μmol/L.

109. The method of claim 108, wherein said COX 2 inhibitor, isomer, pharmaceutically acceptable salt, ester, or prodrug thereof has a COX 1 IC₅₀ of at least about 20 μmol/L.

110. The method of claim 99, wherein said first amount is from about 0.001 to about 100 mg/day per kg of body weight of said subject and said second amount is from about 0.001 to about 100 mg/day per kg of body weight of said subject.

111. The method of claim 110, wherein said first amount is from about 0.05 to about 50 mg/day per kg of body weight of said subject and said second amount is from about 0.1 to about 10 mg/day per kg of body weight of said subject.

112. The method of claim 111, wherein said first amount is from about 1 to about 20 mg/day per kg of body weight of said subject and said second amount is from about 0.5 to about 2 mg/day per kg of body weight of said subject.

113. The method of claim 99 wherein a weight ratio of said first amount to said second amount is from about 0.002 to about 10.

114. The method of claim 113 wherein a weight ratio of said first amount to said second amount is from about 0.1 to about 5.

115. The method of claim 99, wherein said subject is an animal.

116. The method of claim 115, wherein said subject is a human.

117. The method of claim 99, wherein said COX 2 inhibitor or isomer, pharmaceutically acceptable salt, ester, or prodrug thereof and said second drug are administered enterally or parenterally in one or more doses per day.

118. The method of claim 99, wherein said COX 2 inhibitor or isomer, pharmaceutically acceptable salt, ester, or prodrug thereof and said second drug are administered substantially simultaneously.

119. The method of claim 99, wherein said COX 2 inhibitor or isomer, pharmaceutically acceptable salt, ester, or prodrug thereof and said second drug are administered sequentially.
